(19) United States
(12) Patent Application Publication
(10) Pub. No.: US 2003/0198970 A1
(43) Pub. Date: Oct. 23, 2003

(54) Genostics

(75) Inventor: Gareth Wyn Roberts, Cambs (GB)

Correspondence Address: FINNEGAN, HENDERSON, FARABOW, GARRETT & DUNNER, LLP, 1300 I STREET, NW, WASHINGTON, DC, 20005, US

(73) Assignee: GENOSTIC PHARMA LIMITED

(21) Appl. No.: 10/206,568

(22) Filed: Jul. 29, 2002

Related U.S. Application Data
(63) Continuation of application No. 09/325,123, filed on Jun. 3, 1999, now abandoned.

(30) Foreign Application Priority Data
Jun. 6, 1998 (GB) 9812098.3
Dec. 23, 1998 (GB) 9828289.0

(51) Int. Cl. C12Q 1/68; C07H 21/04

(52) U.S. Cl. 435/6; 536/24.3

(57) ABSTRACT

People vary enormously in their response to disease and the also in their response to therapeutic interventions aimed at ameliorating the disease process and progression. However, the provision of medical care and medical management is centered around observations and protocols developed in clinical trials on groups or cohorts of patients. This group data is used to derive a standardised method of treatment which is subsequently applied on an individual basis. There is considerable evidence that a significant factor underlying the individual variability in response to disease, therapy and prognosis lies in a person's genetic make-up. There have been numerous examples relating that polymorphisms within a given gene can alter the functionality of the protein encoded by that gene thus leading to a variable physiological response. In order to bring about the integration of genomics into medical practice and enable design and building of a technology platform which will enable the everyday practice of molecular medicine a way must be invented for the DNA sequence data to be aligned with the identification of genes central to the induction, development, progression and outcome of disease or physiological states of interest. According to the invention, the number of genes and their configurations (mutations and polymorphisms) needed to be identified in order to provide critical clinical information concerning individual prognosis is considerably less than the 100,000 thought to comprise the human genome. The identification of the identity of the core group of genes enables the invention of a design for genetic profiling technologies which comprises of the identification of the core group of genes and their sequence variants required to provide a broad base of clinical prognostic information—‘genostics’. The “GenosticTM” profiling of patients and persons will radically enhance the ability of clinicians, healthcare professionals and other parties to plan and manage healthcare provision and the targeting of appropriate healthcare resources to those deemed most in need. The use of our invention could also lead to a host of new applications for such profiling technologies, such as identification of persons with particular work or environment related risk, selection of applicants for employment, training or specific opportunities or for the enhancing the planning and organisation of health services, education services and social services.

People vary enormously in their response to disease and the also in their response to therapeutic interventions aimed at ameliorating the disease process and progression. However, the provision of medical care and medical management is centered around observations and protocols developed in clinical trials on groups or cohorts of patients. This group data is used to derive a standardised method of treatment which is subsequently applied on an individual basis (e.g. the comment that drugs are often prescribed on the basis that everyone is a 70 kg white male).

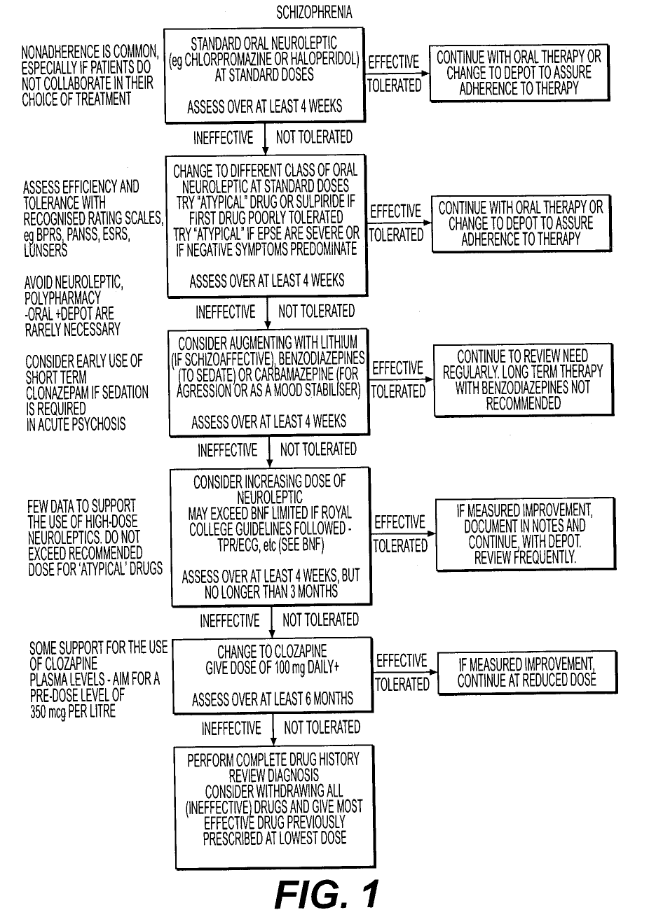

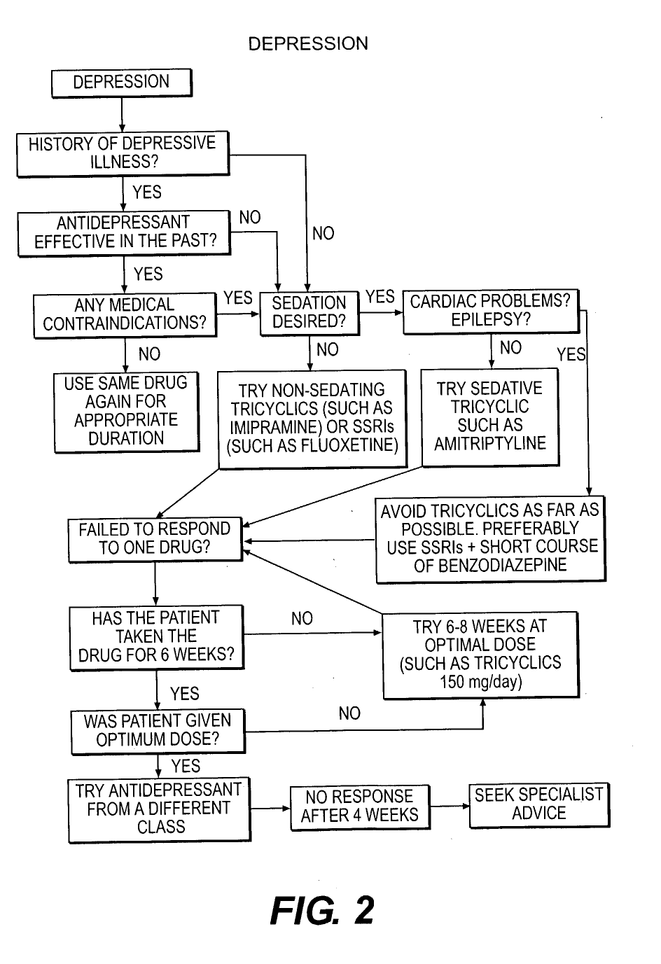

It is standard practice for clinicians to prescribe the same starting dose of a particular drug for a given indication and then adjust the treatment regimen by monitoring the progress of the disease and therapeutic response in individual patients. Observation of actual therapeutic outcome following these adjustments to patient's therapy provides the basis for determining a prognosis for the disease and developing a clinical management plan for patient care (e.g. see FIG. 1, algorithm for management of schizophrenia, from FIG. 1 Taylor and Kerwin 1997, FIG. 2 algorithm for treatment of depression from FIG. 1 Pathare and Paton 1997) and treatment algorithms published by the National Cancer Institute).

The standard practice of clinical management has its disadvantages. In particular it is retro-active in that changes to patient management will occur following the emergence of therapeutic failures, adverse events or other difficulties in undertaking the therapeutic regime (Lazarou et al 1998).

There is considerable evidence that a significant factor underlying this individual variability in response to disease, therapy and prognosis lies in a person's genetic make-up. There have been numerous examples relating that polymorphisms within a given gene can alter the functionality of the protein encoded by that gene thus leading to a variable physiological response (see Marshall 1997a and b for reviews).

Gene sequence variations that are present at a frequency of less than 1% in the population are arbitrarily designated as mutations whilst those at a higher frequency are known as polymorphisms (Schafer and Hawkins 1998).

DNA variants leading to monogenic diseases (e.g. presenilin mutations causing Alzheimer's disease, BRCA mutations causing breast cancer) are usually rare in a population due to the process of natural selection. However, variants of genes involved in, or contributing to, polygenic diseases do not act alone to produce the phenotype. As such selection against them occurs only when they are in the appropriate condition to cause the disease, as a result of this differential selection pressure they the individual variants can exist at quite high frequencies within a population.

Alteration of a single gene may not by itself be detrimental, but in combination with certain variants of other genes, may contribute to a disease phenotype (e.g. el-Zein et al, 1997, observed that the inheritance of a particular combination of metabolising genes is strongly associated with lung cancer). The interaction of the relevant variant genes may be enough to cause a disease phenotype or spectrum of phenotypes, but in many cases other kinds of factors will also influence the course of events (e.g. interaction of ApoE genotype and head injury in Alzheimer's disease Nicholl et al 1996).

The identification of modifier genes that influence the penetrance and expressivity of these risk alleles will be key variables in assessing individual risk profiles. It is likely that the combination of and interaction between small discrete genetic influences on a disease state represent the single largest explanation for the phenotypic variation seen in medicine.

This opens the possibility that the identification of the genes associated with disease and an understanding of how these genes interact with the environment, can lead to better prediction of the outcome of both the disease and the therapeutic process. This in turn would allow the tailoring of resources and therapy to meet the likely requirements of the individual patient (Marshall 1997a). The net result should be improved clinical management, identification of the potential for prevention, the reduction of the burden of disability and, ultimately, improved quality of life for the individual (Poste 1998).

As a result of the appreciation of the contribution of genetic variation to medicine, considerable effort has been made to determine how individual genetic variations affect overall health (including predisposition to disease) and once disease is manifest, the likely patterns of progression, responsiveness to treatment and overall prognosis.

In a quest to understand and plot the limits of genetic variation in humans the Human Genome Project was launched in 1990 with a mission to sequence the code of all 100,000 or so human genes by 2002.

As a result of the Human Genome project not only is the mapping and sequencing of the human genome becoming well understood but also the degree of variability in gene sequence between individuals is being documented (Lander 1996). The average difference between individuals appears to be around 0.3% which equates roughly to a difference in one base pair every 500-1000 base pairs of sequence. The variations are known as polymorphisms and such polymorphic variation is thought underlie much of the clinical variability observed in patients with disease and in their response to therapy.

The resultant explosion of genetic sequence information has lead to the emerging sciences of genomics and proteomics. Within the disciplines technologies have evolved (e.g. polymerase chain reaction, single strand conformational polymorphism etc) which allow us to read individual sequence data and detect and identify polymorphic variation in individuals, in disease states and in different ethnic groups (Griffin et al 1997, Little et al 1997).

As a result of such studies individual genes have been identified which indicate a predisposition to disease or a susceptibility to adverse drug responses (e.g. presenilin gene mutations and development of Alzheimer's disease, BRCA gene mutation and development of breast cancer, ACE polymorphisms and early onset heart disease, cytochrome P450 polymorphisms and drug metabolism).

However, such studies have been completed as academic exercises in scientific discovery and involve individual genes and large groups of patients.

Usually a particular individual response to disease or therapy is likely to result from a complex interaction between multiple genes, discrete environmental factors and the particular therapeutic approach offered (for example see algorithms in FIGS. 1 and 2).

As a result, despite the many publications concerning the theoretical or potential applications of genomics to medicine (e.g. Marshall 1997a and b, Poste 1998, Crooke 1998), progress in implementing these approaches on a practical level has been exceedingly slow. In particular, little progress has been made in the understanding of or the ability to prognose individual response to particular disease states or therapeutic regimes (Poste 1998).

In part this has been related to the types of technology available for such studies (Marshall and Hodgson 1998). Such techniques as MALDI-TOF (Griffin et al 1997), sequencing (Dramanac et al 1998) and molecular beacons (Tyagi et al 1998) are complex and relatively slow and require the availability of specialised laboratories and highly trained personnel.

In recent reviews of the field it has been stated that:

‘within next 10 years when not only all genes (will have been) identified but all common intragenic variation also’ (Lander 1996).

the ‘assembly of comprehensive clinical databanks and their use for large-scale genetic association studies to define robust disease-gene risk correlations’ constitutes a significant technological challenge (Poste 1998).

‘if all human DNA variants were known this set would include all functional polymorphisms and if they could be analysed in all individuals comparison of phenotypes and correlation with genotype might make possible the assignment of function to every gene that predisposes to disease of any kind, and also to nonclinical phenotypes including behavioural traits. The sheer task of this is overwhelming and may never be practical’ (Shafer and Hawkins 1998).

On the basis of the current state of the art it seems clear that translating the colossal investment in the human genome project into a means of revolutionising healthcare management requires both substantial creativity in the harnessing of technologies and considerable technical invention before its promise of can be realised.

For the realisation of the promised revolution in medicine two key factors require consideration;

The human genome is made up of some 100,000 separate genes.

Not all genes are of equal biological importance as regards the physiological functioning of humans.

The first issue, that of reading and tracking the volume of information encapsulated in the human genome by the sequence of 100,000 genes and their mutations and polymorphic variations, is beginning to be addressed by emergent technologies such as DNAchips, MALDI-TOF MS (Marshall and Hodgson 1998 see Table 1) and PEDIAT-type technologies (Fox 1998).

TABLE 1
The main features of some hybridization array formats currently available
(Marshall & Hodgson 1998)

| Company | Arraying method | Hybridization step | Readout | Main focus |
|---|---|---|---|---|
| Affymetrix | On-chip | 10,000-260,000 oligo | Fluorescence | Expression profiling, |
| (Santa Clara, | photolithographic | features probed with | | polymorphism analysis, |
| CA) | synthesis of −20-25- | labelled 30-40 | | and |
| | mer oligos onto | nucleotide fragments | | diagnosis |
| | silicon | of sample cDNA or | | |
| | wafers, which are | antisense RNA | | |
| | diced | | | |
| Brax | Short synthetic oligo, | 1,000 oligos on a | Mass | Diagnostics, expression |
| (Cambridge, | synthesized off chip | “universal chip” | spectrometry | profiling, novel gene |
| UK) | | probed with tagged | | identification |
| | | nucleic acids | | |
| Hyseq | 500-2000 nt DNA | 64 sample cDNA | Radioisotope | Expression profiling, |
| (Sunnyvale, | samples printed onto | spots probed with | | novel |
| CA) | 0.6 | 8,000 7-mer oligos | | gene identification, and |
| | cm² (HyGnostics) or | (HyGnostics) or | | large scale sequencing |
| | ˜18 cm² (Gene | ≦55,000 sample | | (Gene |
| | Discovery) | cDNA spots probed | | Discovery array), |
| | membranes | with 300 7-mer oligos | | polymorphism analysis |
| | | (Gene Discovery) | | and |
| | | | | diagnostics (HyGnostics/ |
| | | | | HyChip arrays), and |
| | | Universal 1024 oligo | Fluorescence | large |
| | | spots probed 10 kb | | sample sequencing |
| | Prefabricated 5-mer | sample cDNAs, | | (HyChip |
| | oligos printed as 1.15 | labelled 5-mer oligos | | array) |
| | cm² arrays onto glass | and ligase | | |
| Incyte | Piezoelectric printing | ≦(eventually 10,000) | Fluorescence and | Expression profiling |
| Pharmaceuticals | for spotting PCR | oligo/PCR fragment | Radioisotope | Polymorphism analysis, |
| (Palo Alto, CA) | fragments and on-chip | spots probed with | | Diagnostics |
| | synthesis of oligos | labelled RNA | | |
| Molecular | 500-5000 nt cDNAs | ˜10,000 cDNA spots | Fluorescence | Expression profiling and |
| Dynamics | printed by pen onto | probed with 200-400 | | novel gene identification |
| (Sunnyvale, | ˜10 | nt labelled sample | | |
| CA) | cm² on glass slide | cDNAs | | |
| Nanogen | Prefabricated ˜20 mer | 25, 64, 100, 400 (and | Fluorescence | Diagnostics and short |
| (San Diego, CA) | oligos, captured onto | eventually 10,000) | | tandem |
| | electroactive spots on | oligo spots polarized | | repeat identification |
| | silicon wafer, which | to enhance | | |
| | are | hybridization to 200- | | |
| | diced. Into ≦1 cm² | 400 nt labelled sample | | |
| | chips | cDNAs | | |
| Protogene | On-chip synthesis of | ≦8,000 oligo spots | Fluorescence | Expression profiling, and |
| Laboratories | 40-50-mer oligos onto | probed with 200-400 | | polymorphism analysis |
| (Palo Alto, CA) | 9 | nt labelled sample | | |
| | cm² glass chip via | nucleic acids | | |
| | printing to a surface- | | | |
| Sequenom | Off-set printing of | 250 locations per | Mass | Novel gene |
| (Hamburg, | array, around 20-25- | SpectroChip | spectrometry | identification, |
| Germany and | mer | interrogated by laser | | candidate gene |
| San | | desorbtion and mass | | validation, |
| Diego, CA) | | spectrometry | | diagnostics, and mapping |
| Synteni | 500-5000 nt cDNAs | ≦10,000 cDNA spots | Fluorescence | Expression profiling and |
| (Fremont, CA) | printed by tip onto ˜4 | probed with 200-400 | | novel gene identification |
| | cm² glass chip | nt labelled sample | | |
| | | cDNAs | | |
| The German | Prototypic DNA | Around 1000 spots on | Fluorescence/mass | Expression profiling and |
| Cancer Institute | macrochip with on- | a 8 × 12 cm chip | spectrometry | diagnostics |
| (Heidelberg, | chip | | | |
| Germany) | synthesis of probes | | | |
| | using f-moc or t-boc | | | |
| | chemistry | | | |

These new technologies mark a significant advance in the potential application of genomic information to the problems of biology and human health. The reason for this is their capability of determining or confirming a large volume of DNA sequence data very quickly at the individual level. In this way they open the door to the application of genomic information to the individual patient.

These technologies are also evolving quickly according to Moore's Law (which posits that computer chips' power doubles every 18 months). For instance, three years ago the genechips made by leading companies held some 20,000 DNA probes. Currently genechips with 65,000 probes are available, and a chip with 400,000 probes has recently been produced (Marshall and Hodgson 1998). Applications for such technologies have included sequencing, diagnostics (mutation detection in the BRCA1 gene for cancer), gene discovery, gene expression profiling and gene mapping (Marshall and Hodgson 1998).

However despite their value as research and diagnostic tools, the genechips in existence are utilized largely as research tools (Marshall and Hodgson 1998). They have not been used as a tool for the express purpose of improving healthcare management by enabling the process of clinical prognosis and facilitating the generation of health risk profiles.

The reason for this is the failure to conceive of or invent an appropriate design which identifies the critical core of genes which are the most important in terms of human function. The genetic variability in this group of genes is the most important contributor to the variation in clinical and physiological phenotypes. Not all genes are equally important in the normal physiological functioning of the human body nor in the induction, development or progression of diseases or physiological states. In a given disease, as few as 5-10 genes in different configurations may be of seminal importance in determining the vast bulk of inter-individual variability to disease and therapeutic approaches (Drews 1997, Goodman and Gillman 1996).

As such, a device capable of delivering information on 10,000 genes may leave its user in grave danger of information overload and render him/her unable to identify and abstract the critical information required to enhance patient management or healthcare.

As a result, the translation of such technologies in genechip devices from research tools into healthcare management tools is severely limited (Marshall and Hodgson 1998, Poste 1998, Schafer and Hawkins 1997).

In an effort to overcome this difficulty a consortium of academic and industrial groups (SNP Consortium) has been formed to try and identify the important disease related variants of human genes. The technologies to be used are the generation and assembly of a SNP map spanning the whole human genome and its application to linkage studies.

However, this approach is still in its infancy and is widely held to face considerable technical hurdles in the robust statistical analysis of huge datasets.

In order to bring about the integration of genomics into medical practice and enable design and building of a technology platform which will enable the everyday practice of molecular medicine a way must be invented for the DNA sequence data to be aligned with the identification of genes central to the induction, development, progression and outcome of disease or physiological states of interest:

Practitioners of molecular healthcare need to be able to;

Identify the presence or absence of a selected group of genes and polymorphic variants central to the induction, development progression and outcome of disease or physiological states

Focus on polymorphisms that lie within the coding or regulatory regions of the gene and are likely to result in altered structure or expression of the protein.

Utilise the data on the core group of genes in order to generate guidelines and guidance for the healthcare management of patients or persons.

The invention described herein identifies the core group of genes required for the design development and manufacture of such a valuable aid to clinical management of the patient and general healthcare management.

According to the invention, the number of genes and their configurations (mutations and polymorphisms) needed to be identified in order to provide critical clinical information concerning individual prognosis is considerably less than the 100,000 thought to comprise the human genome.

The identification of the identity of the core group of genes enables the invention of a design for genetic profiling technologies which comprises of the identification of the core group of genes and their sequence variants required to provide a broad base of clinical prognostic information—‘genostics’.

By careful and lengthy research of the literature, tabulation of data, cross referencing of studies and conduction of a variety of experiments we have identified the core group of genes, which, if assessed for the presence of their functional variants, will enable an enhanced prognosis for an individual patient and form the basis for converting genetic profiling technologies from research tools into universal tools for health management.

Identification of the core group of genes and their functional variants also allows for said technologies to be utilised in generating individual health-risk profiles and profiling the health-risks of the population at large. The determination and identification of sequence data required to identify the important functional variants is readily accomplished by those skilled in the practice of the relevant arts.

The invention does not provide a method for treatment as such. Nor does it provide a direct method of diagnosis of illness or health risk as such. Information obtainable using the invention can be used by a medical practitioner to tailor resources and therapy to meet the likely requirements of individual patients and selected populations of patients. For example in a complex regime or clinical management plan (as seen for example in FIGS. 1 and 2) the invention allows the better prediction of the outcome of both the disease and the chosen therapeutic process.

The enablement of the invention and the generation of the information required for the design of ‘genostics’ requires:

1. Identification of sequence data (Example 1).

2. Assessment of the type and significance of sequence variation in the core group of genes (Examples 2, 3, 4).

3. Identification of likely genetic variation/disease relationships (Example 5 and 5a).

4. Means of identifying and detecting additional polymorphisms in the core group of genes (Example 6).

5. A practical approach to data analysis to generate information on prognosis (Example 7).

6. An illustration of how clinical management of a patient can be enhanced by utilising genetic profiling approaches (Example 8 and 9).

### EXAMPLE 1

Gene sequence data is readily available in the public domain.

For the design of the GENOSTIC genechip device, gene sequence data can be retrieved, by persons skilled in the art, by searching the following public databases:

| Website | Address | Description |
|---|---|---|
| DbEST | http://www.ncbi.nlm.nih.gov/dbEST | Database of expressed |
| | | sequence tags |
| EBI/EMBL | http://www.ebi.ac.uk/mutations/ | Mutations |
| EBI: The European | http://www.ebi.ac.uk/ebi home.html | Nucleotide Sequence |
| Bioinformatics | | Database |
| Institute, | | |
| Hinxton, UK | | |
| EMBL | http://www.ebi.ac.uk/queries/queries.html | Nucleotide Sequence |
| | | Database |
| GDB: The Genome | http://www.gdb.org/gdb/gdbtop.html | Human Genome Database |
| Database, Infobiogen | | |
| European Node, | | |
| France | | |
| GeneCards | http://bioinformatics.weizmann.ac.il/cards/index.html | GeneCards is a database of |
| | | human genes, their products |
| | | and their involvement in |
| | | diseases. |
| GeneClinics | http://www.geneclinics.org/ | GeneClinics (formerly |
| | | Genline) is a knowledge base |
| | | of expert-authored, up-to-date |
| | | information relating genetic |
| | | testing to the diagnosis, |
| | | management, and counseling |
| | | of individuals and families |
| | | with inherited disorders. |
| Genethon | http://www.genethon.fr/genethon_en.html | The Human Genome Research |
| | | Centre. |
| GSDB: Genome | http://www.ncgr.org/ | A collection of DNA |
| Sequence database | | sequence |
| | | data and related information. |
| HGP: Human | http://www.ornl.gov/TechResources/Human_Genome/home.html | Useful background & links. |
| Genome | | |
| Project Information | | |
| Human Gene | http://www.uwcm.ac.uk/uwcm/mg/search | Mutations |
| Mutation | | |
| Database | | |
| NCBI | http://www.ncbi.nlm.nih.gov/ | KEY SITE. Nucleotide |
| | | Sequence retrieval start point |
| OMIM: Online | http://www.ncbi.nlm.nih.gov/Omim/ | This database is a catalog of |
| Mendelian Inheritance | | human genes and genetic |
| in | | disorders. |
| Man | | |
| PubMed | http://www.ncbi.nlm.nih.gov/PubMed/ | PubMed accesses MEDLINE |
| | | medica literature database and |
| | | links to full-text journals. It is |
| | | also the literature component |
| | | of the Entrez retrieval system |
| | | for molecular biology |
| | | information. |
| Research Tools | http://www.ncbi.nlm.nih.gov/SCIENCE96/ResTools.html | A Gene Map of the Human |
| (Science - NCBI) | | Genome. |
| RHdb: Radiation | http://www.ebi.ac.uk/RHdb | Radiation Hybrid Database. |
| Hybrid Database, | | |
| Hinxton, UK | | |
| Stanford Human | http://www.shgc.stanford.edu/ | Sequence database. |
| Genome Centre | | |
| HUGO: The Human | http://www.gene.ucl.ac.uk/hugo | HUGO is the international |
| Genome Organisation | | organisation of scientists |
| | | involved in the Human |
| | | Genome Project. |
| TIGR: The Institute | http://www.tigr.org/ | Genomic databases. |
| for Genomic Research | | |
| The National Human | http://www.nhgri.nih.gov/ | Access to sequence databases |
| Genome Research | | |
| Institute | | |
| The Whitehead | http://www.genome.wi.mit.edu/ | Genome map and sequence |
| Institute Center for | | information. |
| Genome Research | | |
| Unigene: Unique | http://www.ncbi.nlm.nih.gov/UniGene/index.html | UniGene is a system for |
| Human Gene | | automatically partitioning |
| Sequence | | GenBank sequences into a |
| Collection. (NCBI) | | non-redundant set of gene |
| | | oriented clusters. Each |
| | | UniGene cluster contains |
| | | sequences that represent a |
| | | unique gene, as well as related |
| | | information such as the tissue |
| | | types in which the gene has |
| | | been expressed and map |
| | | location. |
| University of | http://dnal.chem.ou.edu/index.html | Genomic databases |
| Oklahoma | | |
| WEHI, Melbourne, | http://wehih.wehi.edu.au/srs/srsc/ | Sequence Retrieval System |
| Aus | | |

Genes coding for proteins known to play a key role in organ function or disease are designated ‘candidate genostic genes’. Variations within the gene structure may alter the regulatory or structural integrity of the gene product leading to enhancement or reduction in the specific function (e.g. receptor binding, enzyme activity). The exact role that a candidate gene plays in disease, prognosis and healthcare management can be fully ascertained by assessing the effects of variation in gene structure in particular patient groups, populations or individuals (see examples 2, 3 and 4).

### EXAMPLE 2

### Candidate Genostic Genes

Human Neuronal Nitric Oxide Synthetase

Gene Map Locus: 12q24.2q24.31(OMIM Ref. 163731).

One candidate ‘genostic’ gene is the gene encoding nitric oxide synthetase (NOS-1).

The enzymes responsible for NO synthesis in man constitute a family with at least three distinct isoforms: inducible, endothelial, and neuronal. Neuronal NO synthetase (NOS-1) is localised to human chromosome 12, and participates in diverse biologic processes including neurotransmission, the regulation of body fluid homeostasis, neuroendocrine physiology, control of smooth muscle motility, sexual function and monocyte biology.

Burnett et al. (1992) localized NO synthase to rat penile neurons innervating the corpora cavernosa and to neuronal plexuses in the adventitial layer of penile arteries. They demonstrated that small doses of NO synthase inhibitors abolished electrophysiologically induced penile erections establishing that nitric oxide is a physiologic mediator of erectile function.

Kharazia et al. (1994) found that all neurons in the striatum and many in the cortex were positive for nitric oxide synthase indicating a role of NOS in brain function.

NOS1 cDNA clones contain different 5-prime terminal exons spliced to a common exon 2. Xie et al. (1995) demonstrated that the unique exons are positioned within 300 bp of each other but separated from exon 2 by an intron that is at least 20 kb long. A CpG island engulfs the downstream 5-prime terminal exon. In contrast, most of the upstream exon resides outside of this CpG island. The upstream exon includes a GT dinucleotide repeat. The expression of these 2 exons is subject to transcriptional control by separate promoters. Nitric oxide is synthesized in skeletal muscle by neuronal-type NO synthase, which is localized to sarcolemma of fast-twitch fibers. Synthesis of NO in active muscle opposes contractile force. Brenman et al. (1995) showed that NOS1 partitions with skeletal muscle membranes owing to association of enzyme with dystrophin, the protein mutated in Duchenne muscular dystrophy. The dystrophin complex interacts with an N-terminal domain of NOS1 that contains a GLGF motif. Both humans with DMD and mdx mice show a selective loss of NOS1 protein and catalytic activity from muscle membranes. NOS1-deficient mice are resistant to neural stroke damage following middle cerebral artery ligation. Nelson et al. (1995) reported a large increase in aggressive behavior and excess, inappropriate sexual behavior in NOS1 ‘knockout’ mice. Initial observations indicated that male (but not female) NOS 1-deficient mice engaged in chronic aggressive behavior.

Magee et al. (1996) used PCR to clone a novel form of neuronal NOS from rat penile RNA. This NOS cDNA was termed PnNOS for ‘penile neuronal NOS.’ Sequencing revealed that the PnNOS cDNA was identical to rat cerebellar neuronal NOS1 except for a 102-bp insertion in PnNOS. Repetition of RT-PCR showed PnNOS to be the only form of NOS1 expressed in rat penis, urethra, prostate, and skeletal muscle. PnNOS may be responsible for the synthesis of nitric oxide during penile erection and may be involved in control of the tone of the urethra, prostate, and bladder.

Using the available genomic sequence of neuronal NOS-1 it is possible to identify those parts of the gene which show variation sufficient to alter the normal functioning of the gene.

1.) Transcriptional Promoter Sequences:

Sequence mutations in the promoter region of the NOS1 gene will allow the identification of individuals with altered transcriptional regulation control.

2.) RNA Processing (Splicing) Sequences:

Characterise mutations in the intron/exon structure of the NOS1 gene to identify individuals with altered RNA splicing patterns. These results in truncated proteins or splice variants with an altered function.

3.) Messenger RNA Translation and Stability Sequences:

Sequence and characterise mutations within the repetitive sequences located in the 3′ untranslated region of the NOS-1 gene. These individuals have altered translational control of their mRNA.

4.) DNA Sequences Involved in Genomic Rearrangement or Expansion:

The presence of Alu-1 repeat, which are known to cause recombination, allows one to detect gross chromosomal rearrangements. Changes in either the sequence or the genomic structure may well correlate with clinical or pathological symptoms.

102-bp insertion will also be involved in the functional variation of activity involving the urogenital tract.

5.) Coding Sequences:

Mutations and polymorphisms in the coding (exon) sequences of the NOS-1 gene will result in changes at the structural level of the protein with functional changes. Amino acid substitutions, within neuronal NOS-1, will play a role in age/brain related neuronal defects.

The specific sequences are detailed in Table 2.

TABLE 2
Summary of Genome Elements within the Neuronal Nitric Oxide
Synthetase Gene.

| Gene Anatomy | Key Region | Functional Elements |
|---|---|---|
| 1. 5′ Flanking Region: | GC-enriched sequences: | DNA methyltransferase foot print region |
| | | CpG Island |
| | Promoter elements | TATA box |
| | | Inverted CAAT boxes |
| | | AP-2-like element |
| | | CREB/ATF element |
| | | c-Fos element |
| | | NF-kB-like |
| | | ETS-binding sites |
| | | TEF-1/MCBF binding sites |
| | | NRF-1 binding sites |
| | | RNA Pol III site |
| 2. Exon Coding Regions | | Translation initiation exon 2 |
| | | Translation termination exon 29 |
| 3. RNA Processing | | Intron/exon boundaries (1-29) |
| | | Cassette splicing exons 9-11 |
| 4. RNA Translation | | 3′ Untranslated Region |
| 5. Insertion | | 102 bp insertion |
| 6. Repetitive Sequences | | Alu-1 family |
| | | Dinucleotide repeats |

These variations in the genomic structure of the human NOS1 gene are important in controlling the physiological role of NOS in normal or disease states in humans. Alterations in the physiology of NOS have significant healthcare indications (i.e. stroke, cardiac and circulatory disease, urogenital disease and dysfunction, psychiatric symptoms and musculoskeletal disorders).

In consideration with an assessment of the functional variation in other genes, identification of the pattern of NOS1 gene variation in a patient cohort, population or individual offers a powerful practical tool for improving the management of healthcare and the prognosis of health risk.

### EXAMPLE 3

Voltage-Gated Calcium Channels

Gene Map Locus (OMIN Ref.601011)

Other candidate ‘genostic’ genes are the calcium channel subunit genes.

There are six functional subclasses of calcium channel. Voltage-dependent Ca(2+) channels not only mediate the entry of Ca(2+) ions into excitable cells but are also involved in a variety of Ca(2+)—dependant processes, including muscle contraction, hormone or neurotransmitter release and gene expression.

Calcium Channels are multi-subunit complexes and the channel activity is directed by a pore-forming alpha-1 sub-unit. The auxiliary sub-units beta, alpha-2/delta, and gamma regulate channel activity. Ca(2+) currents have been described on the basis of their biophysical and pharmacological properties and include L-, N-, T-, P-, Q-, and R-types.

P/Q type channels colocalise with a subset of docked vesicles at the synapse where they control exocytosis, demonstrated by the sensitivity of various types of neurotransmission to specific blockers of these channels. P/Q type channels are involved in CSD (cortical spreading depression—which causes the aura or visual symptoms of migraine) and release of neurotransmitters, including 5-HT (migraine patients have systemic disturbance of 5-HT metabolism).

The distinctive properties of each of the Ca(2+) channel types are primarily related to the expression of a variety of alpha-1 isoforms (Dunlap et al., 1995). There are at least 6 classes of alpha-1 subunits: alpha-1A, B, C, D, E and S. They are derived from 6 genes representing members of a gene family. The alpha-1A, B and E isoforms are abundantly expressed in the neuronal tissue. The genes encoding the alpha-1A, B, and E isoforms are symbolised CACNL1A4, CACNL1A5, and CACNL1A6 respectively.

The CACNL1A4 gene was assigned to 19p13, (Diriong et al., 1995). The gene was characterised by Ophoff et al (1996) in preparation for a mutation search in neurological disorders that map to 19p13. They found that the gene covers 300 kb with 47 exons and reported the amino acid sequence for residues 1-2262. Sequencing of all the exons and their surroundings revealed polymorphic variations, including a (CA)n-repeat, a (CAG)n-repeat in the 3-prime-UTR, and different types of deleterious mutations in 2 neurological disorders; familial hemiplegic migraine and episodic ataxia type 2. Thus, these 2 neurological disorders are allelic channelopathies.

Calcium channels are also known to be important in regulating the function of the heart (particularly arrhythmias) and a number of drugs express their therapeutic effects by blocking myocardial Ca(2+) or prolonging the activation time of the channel (Brody, Larner and Minneman 1998). Polymorphic variation can help predict individual response to injury and disease, the symptoms and consequences of cardiovascular disease, dysfunction and damage to the system.

### EXAMPLE 4

Lipoprotein Lipase LPL

Gene Map Locus (OMIN Ref. 238600)

A third example of a candidate for a ‘genostic’ gene is the enzyme lipoprotein lipase (LPL).

Human lipoprotein lipase is a member of a lipase gene family, which also includes the hepatic and pancreatic lipases. LPL is located on the surface of endothelial cells of capillaries where it hydrolyses triacylglycerols of plasma lipoproteins to fatty acids and glycerol. These fatty acids are then taken up by cell and used for energy production. The enzyme plays a central role in lipid metabolism and is a candidate susceptibility gene for cardiovascular disease.

The LPL gene contains ten exons spanning 30 kb and encodes a protein of 475 amino acids and has several well characterised functional domains including the APOC-II binding site, the heparin-binding clusters used to localise LPL to the endothelial wall and the domains that contribute to the active site.

Diseases that affect the metabolism and transport of lipids frequently result in abnormally high plasma triacyglycerols and or cholesterol that are often associated with coronary artery disease, artherosclerosis and/or obesity. DNA sequence variation in genes that encode many of the enzymes and proteins involved in lipid metabolism and transport (including LPL) have been identified and associated with clinically abnormal lipid profiles.

The LPL gene sequence has been shown to contain distinct sequence variations among populations, (Nickerson et al, 1998). Nickerson et al described 88 variants in a region of the LPL gene, 90% of which were single nucleotide polymorphisms (SNPs), the remaining being insertion-deletion variations. 81 variants were found in intronic regions, and 7 in the exonic sequence. Only 4 of the exonic variants altered the protein sequence.

Assessing the functional variability of the LPL gene in conjunction with the functional variabilty of other core genes will provide a tool in predicting the likelihood of developing a range of diseases including the symptoms and consequences of coronary artery disease, artherosclerosis and/or obesity.

As shown above, sequence data for genes of interest can be readily obtained. Genetic variation in specific regions of genes can also be determined. The identification of a core group of genes which have important effects on the key physiological and pathophysiological processes in human disease would form an important medical advance.

A device or detector configured and designed using this core group of genes (GENOSTIC) would have a general utility in the practice of medicine and healthcare management for:

prognosing the course of illness

predicting likely therapeutic response

identifying potential adverse event profile.

### EXAMPLE 5

List of Genes with Known Association with Disease

The following are examples of genes with known associations with disease which can be discerned by a careful review of the medical and biochemical literature and by experimentation. Many such genes can also be identifed by a review of publicly available databases e.g. Human Gene Mutation Database

(http://www/uwcm.ac.uk/uwcm/mg/search/), OMIM Database

(http://www.ncbi.nlm.nih.gov/omim) or GENECARDS

(http://bioinformatics.welzmann.ac.il/cards/index.html).

Note: The tabulated genes are listed in alphabetical groups, but the numbering of genes within each group is not necessarily continuous.

| A | B | C | D |
|---|---|---|---|
| 1:APOA4 | 1:BLM | 1:CRYAA | 1:DPYD |
| 2:AAC2 | 2:BCKDHA | 2:CRYBB2 | 2:DIAPH1 |
| 3:AD2 | 3:BTD | 3:CHM | 3:DMD |
| 4:AGA | 4:BPGM | 4:C2 | 4:DPYS |
| 5:APOA1 | 5:BRCA2 | 5:C5 | 5:DFN1 |
| 6:ALAS2 | 6:BRCA1 | 6:C9 | 6:DKC1 |
| 7:ALB | 7:BCP | 7:C3 | 7:DLD |
| 8:APT1 | 8:BLMH | 8:C7 | 8:DENA5 |
| 9:APOA2 | 9:BCKDHB | 9:CTNS | 9:DTD |
| 10:APOH | 10:BCHE | 10:C1QA | 10:DCX |
| 11:AMELX | 12:BTK | 11:C1QB | 11:DYT1 |
| 12:APT1LG1 | 13:BARD1 | 12:CNGA3 | 12.DMPK |
| 13:A2M | 18:BSEP | 13:C1QG | 13:DRD4 |
| 14:APBB1 | | 14:CPO | 14:DDB2 |
| 15:AGXT | | 15:CDH1 | 15:DIAPH2 |
| 16:AGTR1 | | 16:C4A | 16.dgcr5 |
| 17:ALDH2 | | 17:C4B | 17:DRD2 |
| 18:ARG1 | | 18:C6 | 18:DES |
| 19:ALD | | 19:C8B | 19:DBT |
| 20:AGT | | 20:CACT | 20:DCP1 |
| 21:ACHE | | 21:chit | 24:DYSF |
| 22:ADSL | | 22:CLCN1 | 27:DRA |
| 23:ADRB3 | | 23:CFTR | 29:DLX3 |
| 24:atpsk2 | | 24:COL1OA1 | 31:DRPLA |
| 25:ATM | | 25:CYP1A1 | 38:DIA1 |
| 26:ASPA | | 26:CLCNKB | 39:DHAPAT |
| 27:ACTC | | 27:CD3G | |
| 28:ADRB2 | | 28:CAGNA1F | |
| 29:AIRE | | 29:CPS1 | |
| 30:AZF1 | | 30:CRX | |
| 31:AT3 | | 31:CYBA | |
| 32:ABO | | 32:CKN1 | |
| 33:ABCR | | 33:CST3 | |
| 34:AACT | | 34:CNGA1 | |
| 36:ANK1 | | 35:CETP | |
| 37:ALAD | | 36:CAT | |
| 38:APOE | | 37:CTSK | |
| 39:APP | | 38:CYBB | |
| 40:APOC3 | | 40:CSX | |
| E | F | G | H |
| 1:EPOR | 1:FUCA1 | 1:GM2A | 2:HD |
| 2:EPB41 | 2:FRDA | 2:GYPC | 3:HK1 |
| 3:EMX2 | 3:FGB | 3:GALT | 5:HBG2 |
| 4:EXT2 | 4:EH | 4:GLB1 | 6:HSD3B2 |
| 5:EMD | 5:FGG | 5:GALE | 7:HBG1 |
| 6:ED1 | 6:FMR2 | 6:GAMT | 9:HFE |
| 7:ESR | 7:FGFR1 | 7:GYPA | 10:HTN3 |
| 8:EXT1 | 8:FGA | 8:GPI | 11:HOXA 13 |
| 9:EPHX1 | 9:F10 | 9:GPC3 | 12:HR |
| 10:EPX-PEN | 10:FUT6 | 10:GLI3 | 13:HBA1 |
| 11:EDNRB | 11:FKHL15 | 11:GCDH | 14:HMGCL |
| 12:EPM2A | 12:FRAXF | 12:GAA | 15:HBD |
| 13:EDN3 | 13:FBP1 | 13:G6PC | 16:HTR2C |
| 14:ETFA | 14:F11 | 14:GBA | 18:HP |
| 15:ETFB | 15:F12 | 15:GALK1 | 19:HSD11B2 |
| 16:ENG | 16:FCGR1A | 16:GBE1 | 20:HK2 |
| 17:EPB42 | 17:FBN2 | 17:GLS | 21:HPS |
| 18:ETFDH | 18:FAH | 18:G6PT 1 | 23:HGD |
| 19:EFE2 | 19:FSHR | 19:GLUD1 | 25:HBA2 |
| 20:ERCC5 | 20:F13B | 20:GRL | 26:HCF2 |
| 22:ERCC4 | 21:FMO3 | 21:GSS | 27:HRG |
| 23:ELN | 22:FUT3 | 22:GK | 28:HOXD 13 |
| 24:EYA1 | 23:F13A1 | 23:GP1BB | 29:HEXB |
| 25:ERCC6 | 24:FANCA | 24:GSN | 32:HLCS |
| 26:ERCC3 | 25:F7 | 25:GCGR | 33:HPRT1 |
| 27:EGR2 | 26:FTL | 26:GLRA1 | 34:HBB |
| 28:ERCC2 | 27:F5 | 27:GH1 | 35:HTR1A |
| | 28:FUT2 | 28:G6PD | 36:HSD17B1 |
| | 29:FMR1 | 29:GYS2 | 37:HSD17B3 |
| | 30:FCMD | 30:GHRHR | 40:HSD17B4 |
| | 31:FGDY | 31:GH2 | |
| | 32:FANCC | 32:GCP | |
| | 33:FCGR2A | 33:GALC | |
| | 34:FGFR3 | 34:GP9 | |
| | 35:FECH | 35:GNRHR | |
| | 36:FSHB | 36:GIPR | |
| | 37:F8C | 37:GSTT1 | |
| | 38:FBN1 | 38:GLA | |
| | 39:FABP2 | 39:GRPR | |
| | 40:F9 | 40:GPD2 | |
| I | J | K | L |
| 1:IL2RA | 1:JAG 1 | 1:KRT9 | 1:LPL |
| 2:IVD | 2:JAK3 | 2:KCNQ3 | 2:LJPC |
| 4:IFNGR 1 | | 3:KRT1 | 3:LOR |
| 5:IL2RG | | 4:KNG | 4:LDLR |
| 6:IFNGR2 | | 5:KRT16 | 5:LYZ |
| 7:IGHG2 | | 6:KRT18 | 6:LIG1 |
| 9:INSR | | 7:KRT6A | 7:LDHA |
| 10:IDUA | | 8:KRT6B | 8:LDHB |
| 11:IL4R | | 9:KRT3 | 9:LQT2 |
| 12:ITGA7 | | 10:KHK | 10:LEPR |
| 13:ITGA2B | | 11:KRTHB1 | 11:LHCGR |
| 14:IGKV | | 12:KEL | 12:LEP |
| 15:IAPP | | 13:KRTHB6 | 13:LHB |
| 16:IPF1 | | 14:KAL1 | 14:LIPA |
| 17:INS | | 15:KRT4 | 15:LAMA3 |
| 18:IGF1 | | 16:KRT13 | 16:LICAM |
| 19:IGHM | | 17:KRT2A | 17:LAMC2 |
| 20:ITGA6 | | 18:KRT12 | 19:LCAT |
| 21:IRS1 | | 19:KRT5 | 20:LAMA2 |
| 22:ICAM1 | | 20:KRT14 | 21:LMX1B |
| 23:ITGB3 | | 21:KRT10 | 22:LTBP2 |
| 24:ITGB4 | | 22:KRT17 | 23:LMAN1 |
| 25:IDS | | 23:KCNQ2 | 26:LAMB3 |
| 28:ITGB2 | | 24:KCNQ1 | |
| | | 26:KCNJ 1 | |
| | | 28:KCNJ11 | |
| | | 30:KCNA1 | |
| | | 32:KIT | |
| | | 36:KCNE1 | |
| M | N | O | P |
| 1:MTM1 | 1:NME1 | 1:OA1 | 1:PROP1 |
| 2:MUT | 2:NF1 | 2:OCA2 | 2:PLP |
| 3:MTR | 3:NBS1 | 3:OCRL | 3:PRPS1 |
| 4:MLH 1 | 4:NPHP1 | 4:OXCT | 4:PEPD |
| 5:MMP3 | 5:NF2 | 5:OPHN1 | 5:PCCB |
| 6:MVK | 6:NCF1 | 6:OTC | 6:PCCA |
| 7:MANBA | 7:NDP | 7:OAT | 7:PCSK1 |
| 8:MTRR | 8:NCF2 | 8:COLIA2 | 8:PAH |
| 9:MANB | 9:NP | | 9:POU1F1 |
| 10:MPO | 10:NEU | | 10:PPOX |
| 11:MYO5A | 11:NTF3 | | 11:PRKCG |
| 12:MYH7 | 12:NOTCH3 | | 12:PXMP1 |
| 13:MAOA | 13:NRTN | | 13:PPGB |
| 14:MYOC | 14:CHRNA4 | | 14:PRB3 |
| 15:MADH4 | 15:NPC1 | | 15:PRB1 |
| 16:MEFV | 16:NAGA | | 16:PRB4 |
| 17:MAT1A | 17:NEFH | 17:PMP22 | |
| 18:MEN1 | 18:NTRK1 | | 18:PABP2 |
| 19:MOCS1 | 19:NAIP | | 19:PEX7 |
| 20:mocs1b | 20:NDUFS4 | | 20:PDDR |
| 21:MLR | 21:NOS3 | | 21:PAFAH2 |
| 22:MSH2 | 23:NODAL | | 22:PARK2 |
| 23:MSX2 | 25:NAGLU | | 23:PLG |
| 25:MPI | | | 24:PPARG |
| 26:MC4R | | | 25:PON2 |
| 28:MDCR | | | 26:PROC |
| 29:MBL | | | 27:PROS1 |
| 30:MJD | | | 28:PDE6A |
| 31:MC2R | | | 29:PXMP3 |
| 32:MYL2 | | | 30:PPP1R3 |
| 33:MC1R | | | 31:PON 1 |
| 34:MYO15 | | | 32:PEX1 |
| 35:MAPT | | | 33:PC |
| 36:MPZ | | | 34:PENK |
| 37:MID1 | | | 35:PXR1 |
| 38:MSX1 | | | 36:PGK1 |
| 39:MGAT2 | | | 37:PTH |
| 40:MTHFR | | | 38:PDE6B |
| | | | 39:PSEN2 |
| | | | 40:PKD2 |
| Q | R | S | T |
| 1:QDPR | 1:RHO | 1:SSA1 | 1:TAT |
| | 2:RP2 | 2:SOD1 | 2:THBD |
| | 3:RLBP1 | 3:COL2A1 | 3:TNNT2 |
| | 4:RHD | 4:SDH2 | 4:TF |
| | 5:RBI | 5:SGSH | 5:TBG |
| | 6:ROM1 | 6:SLC5A5 | 6:TSC1 |
| | 7:RP3 | 7:SLC12A3 | 7:TCN2 |
| | 8:RHCE | 8:SDH1 | 8:TP11 |
| | 9:RHAG | 9:SUOX | 9:TPM1 |
| | 10:RHOK | 10:STS | 10:TBXA2R |
| | 12:rfxank | 11:ssadh | 11:TPMT |
| | 13:REN | 12:SALL1 | 12:TYR |
| | 14:RYR1 | 13:SHOX | 13:TGM1 |
| | 15:RS1 | 14:SLC12A1 | 14:TTR |
| | 16:RDS | 15:SLC2A2 | 15:TSC2 |
| | 17:RFC2 | 16:SNRPN | 16:TG |
| | 18:RCP | 17:SPTB | 17:TTPA |
| | 21:RFXAP | 18:SCA2 | 18:TCOF1 |
| | 22:RAG2 | 19:SMN1 | 19:TULP 1 |
| | 23:RPS6KA3 | 20:STK11 | 20:TNF |
| | 24:RPE65 | 21:SPTA1 | 21:THPO |
| | 25:RFX5 | 23:SH2D1A | 22:TCF2 |
| | 26:RAG1 | 24:SCNN1B | 23:TPO |
| | | 25:SI | 24:TEK |
| | | 26:SCA1 | 25:TPM3 |
| | | 27:SLC2A1 | 26:TYRP1 |
| | | 28:SELE | 27:TGFB1 |
| | | 31:SAA1 | 28:TSHB |
| | | 32:SNCA | 29:TNN13 |
| | | 33:SOD3 | 30:TIMP3 |
| | | 34:SCN1B | 31:TECTA |
| | | 35:SLC6A4 | 32:TAP1 |
| | | 36:SRK | 33:TCF14 |
| | | 37:SLC5A1 | 36:TH |
| | | 39:SLC10A2 | 37:TSHR |
| | | | 38:THRB |
| | | | 39:TAP2 |
| | | | 40:TGFBR2 |
| U | V | W | X |
| 1:UMPS | 1:VWF | 1:WT1 | 1:XPA |
| 2:UGB | 2:VDR | 2:WFS1 | 2:XDH |
| 3:USH2A | 3:VMD2 | 3:WRN | 3:XPC |
| 4:UFD1L | 4:VHL | 4:WAS | 6:XK |
| 5:ugtld | | | 8:X1ST |
| 6:UROD | | | 9:XRCC9 |
| 7:UBE3A | | | |
| 8:UCP3 | | | |
| 9:UROS | | | |
| 10:UGT1 | | | |
| Y | Z | | |
| | 1:Z1C2 | | |
| | 2:Z1C3 | | |

### EXAMPLE 5a

Polymorphic Variation

For each gene, sequence data concerning the existence of polymorphic variation can be located. For example, below are the details of the polymorphic variations of six genes, representative of major gene product/protein categories on the core list.

Category 1 - Enzymes
α-glucosidasc

| | |
|---|---|
| Mutation type | Total number of mutations |
| Nucleotide substitutions (missense/nonsense) | 20 |
| Nucleotide substitutions (splicing) | 4 |
| Nucleotide substitutions (regulatory) | 0 |
| Small deletions | 7 |
| Small insertions | 0 |
| Small indels | 0 |
| Gross deletions | 1 |
| Gross insertions & duplications | 0 |
| Complex rearrangements (including inversions) | 1 |
| Repeat variations | 0 |
| TOTAL | 33 |

| Accession | | | | |
|---|---|---|---|---|
| Number | Codon | Nucleotide | Amino acid | Phenotype |
| CM970540 | 40 | cCGA-TGA | Arg-Term | Glycogen storage disease 2 |
| CM950491 | 299 | CTG-CGG | Leu-Arg | Glycogen storage disease 2 |
| CM980577 | 309 | cGGG-AGG | Gly-Arg | Glycogen storage disease 2 |
| CM910167 | 318 | ATG-ACG | Met-Thr | Glycogen storage disease 2 |
| CM900102 | 402 | aTGG-CGG | Trp-Arg | Glycogen storage disease 2 |
| CM940798 | 519 | cATG-GTG | Met-Val | Glycogen storage disease 2 |
| CM910168 | 521 | cGAG-AAG | Glu-Lys | Glycogen storage disease 2 |
| CM940799 | 545 | CCT-CTT | Pro-Leu | Glycogen storage disease 2 |
| CM980578 | 566 | cTTC-CCC | Ser-Pro | Glycogen storage disease 2 |
| CM930287 | 643 | eGGG-AGG | Gly-Arg | Glycogen storage disease 2 |
| CM940800 | 645 | GACg-GAA | Asp-Glu | Glycogen storage disease 2 |
| CM980579 | 645 | cGAC-AAC | Asp-Asn | Glycogen storage disease 2 |
| CM950492 | 645 | eGAC-CAC | Asp-His | Glycogen storage disease 2 |
| CM940801 | 647 | TGCg-TGG | Cys-Trp | Glycogen storage disease 2 |
| CM980580 | 648 | eGGC-AGC | Gly-Ser | Glycogen storage disease 2 |
| CM980581 | 672 | CGG-CAG | Arg-Gln | Glycogen storage disease 2 |
| CM980582 | 672 | gCGG-TGG | Arg-Trp | Glycogen storage disease 2 |
| CM930288 | 725 | cCGG-TGG | Arg-Trp | Glycogen storage disease 2 |
| CM980583 | 768 | CCC-CGC | Pro-Arg | Glycogen storage disease 2 |
| CM930289 | 854 | cCGA-TGA | Arg-Term | Glycogen storage disease 2 |

| Accession | | Donor/ | Relative | | |
|---|---|---|---|---|---|
| Number | IVS | Acceptor | location | Substitution | Phenotype |
| CS941486 | 1 | as | −13 | T-G | Glycogen storage disease 2 |
| CS971665 | 6 | as | −22 | T-G | Glycogen storage disease 2 |
| CS941487 | 10 | ds | +1 | G-C | Glycogen storage disease 2 |
| CS971666 | 16 | ds | +2 | T-C | Glycogen storage disease 2 |

| Accession | Location/ | | |
|---|---|---|---|
| Number | codon | Deletion | Phenotype |
| CD981927 | 126 | GCAGCCC\hat{ }TGGtgCTTCTTCCCA | Glycogen storage |
| | | | disease 2 |
| CD972136 | 160 | CACCTTCA\hat{ }TTCccCAAGGACATC | Glycogen storage |
| | | | disease 2 |
| CD941678 | 174 | TGATG\hat{ }GAGACtGAGAACCGCC | Glycogen storage |
| | | | disease 2 |
| CD961963 | 470 | CATCACC\hat{ }AACgagaCCGGCCAGCC | Glycogen storage |
| | | | disease 2 |
| CD941679 | 485 | CGGGTCC\hat{ }ACTgccttccccgactTCACCAACCC | Glycogen storage |
| | | | disease 2 |
| CD981928 | 674 | CGGAAC\hat{ }CACAacaGCCTGCTCAG | Glycogen storage |
| | | | disease 2 |
| CD951684 | 902 | GCAGCTG\hat{ }CAGaagGTGACTGTCC | Glycogen storage |
| | | | disease 2 |

| Description | Phenotype |
|---|---|
| 536 by 117E18-332 to E18119+39 | Glycogen storage disease 2 |
| (mutation described at genomic DNA level) | |
| Description | Phenotype |
| Ins C nt. 2741, ins G nt. 2743 | Glycogen storage disease 2 |

Category 2-Transport and Storage
Albumin

| | Total number of |
|---|---|
| Mutation type | mutations |
| Nucleotide substitutions (missense/nonsense) | 21 |
| Nucleotide substitutions (splicing) | 2 |
| Nucleotide substitutions (regulator) | 0 |
| Small deletions | 2 |
| Small insertions | 1 |
| Small indels | 0 |
| Gross deletions | 0 |
| Gross insertions & duplications | 0 |
| Complex rearrangements (including inversions) | 0 |
| Repeat variations | 0 |
| TOTAL | 26 |

| Accession | | | Amino | |
|---|---|---|---|---|
| Number | Codon | Nucleotide | acid | Phenotype |
| CM910024 | 1 | GAT-GTT | Asp-Val | Albumin variant |
| CM940018 | 3 | aCAC-TAC | His-Tyr | Albumin variant |
| CM910025 | −1 | CGA-CAA | Arg-Gln | Albumin variant |
| CM910026 | −2 | CGT-CAT | Arg-His | Albumin variant |
| CM900011 | −2 | tCGT-TGT | Arg-Cys | Albumin variant |
| CM940019 | 32 | tCAG-TAG | Gin-Term | Analbuminaemia |
| CM940020 | 114 | cCGA-TGA | Arg-Tcrm | Analbuminaemia |
| CM910027 | 128 | CAT-CGT | His-Arg | Albumin variant |
| CM940021 | 214 | TGGg-TGA | Trp-Term | Analbuminaemia |
| CM920015 | 218 | CGC-CAC | Arg-His | Albumin variant |
| CM970070 | 218 | CGC-CCC | Arg-Pro | Dysalbuminaemic hyperthyroxinaemia, |
| | | | | familial |
| CM940022 | 225 | cAAA-CAA | Lys-Gln | Albumin variant |
| CM940023 | 276 | AAGG- | Lys-Asn | Albumin variant |
| | | AAC | | |
| CM940024 | 313 | AAGG- | Lys-Asn | Albumin variant |
| | | AAT | | |
| CM910028 | 365 | GAT-GTT | Asp-Val | Albumin variant |
| CM910029 | 372 | cAAA-GAA | Lys-Glu | Albumin variant |
| CM900012 | 501 | aGAG-AAG | Glu-Lys | Albumin variant |
| CM930016 | 505 | tGAA-AAA | Glu-Lys | Albumin variant |
| CM940025 | 563 | cGAT-AAT | Asp-Asn | Albumin variant |
| CM910030 | 570 | cGAG-AAG | Glu-Lys | Albumin variant |
| CM940026 | 573 | tAAA-GAA | Lys-Glu | Albumin variant |

| Accession | Location/ | | | |
|---|---|---|---|---|
| Number | codon | Deletion | Phenotype | |
| CD941562 | 566 | TAAGGAG\hat{ }ACCtGCTTTGCCGA | Albumin variant | |
| CD910474 | 579 | TGCTGCA\hat{ }AGTcAAGCTGCCTT | Albumin variant | |
| Accession | | | | |
| Number | Nucleotide | Codon | Insertion | Phenotype |
| C1941818 | 9156 | 267 | A | Analbuminaemia |

Category 3 - Structural Proteins
Collagen IV alpha 3

| Mutation type | Total number of mutations |
|---|---|
| Nueleotide substitutions (missense/nonsense) | 2 |
| Nucleotide substitutions (splicing) | 1 |
| Nucleotide substitutions (regulatory) | 0 |
| Small deletions | 2 |
| Small insertions | 0 |
| Small indels | 0 |
| Gross deletions | 0 |
| Gross insertions & duplications | 0 |
| Complex rearrangements (including inversions) | 0 |
| Repeat variations | 0 |
| TOTAL | 5 |

| Accession | | | | |
|---|---|---|---|---|
| Number | Codon | Nucleotide | Amino acid | Phenotype |
| CM940306 | 1481 | aCGC-TGA | Arg-Term | Alport syndrome |
| CM940307 | 1524 | TCA-TGA | Ser-Term | Alport syndrome |

| Accession | | Donor/ | Relative | | |
|---|---|---|---|---|---|
| Number | IVS | Acceptor | location | Substitution | Phenotype |
| CS951356 | 5 | as | −320 | G-T | Alport syndrome |

| Accession | Location/ | | |
|---|---|---|---|
| Number | codon | Deletion | Phenotype |
| CD951631 | 1448 | TTTGTCATTCAcccgacaCAGTCAAACC | Alport syndrome |
| CD941648 | 1471 | AGTGGGTATTTcttttCTTTTTGTAC | Alport syndrome |

Category 4 - Immune Protection and inflammation

| Interleukin 4 receptor | Total number |
|---|---|
| Mutation type | of mutations |
| Nucleotide substitutions (missense / nonsense) | 1 |
| Nucleotide substitutions (splicing) | 0 |
| Nucleotide substitutions (regulatory) | 0 |
| Small deletions | 0 |
| Small insertions | 0 |
| Small indels | 0 |
| Gross deletions | 0 |
| Gross insertions & duplications | 0 |
| Complex rearrangements (including inversions) | 0 |
| Repeat variations | 0 |
| TOTAL | 1 |

| Accession | | | | |
|---|---|---|---|---|
| Number | Codon | Nucleotide | Amino Acid | Phenotype |
| CM970744 | 576 | CAG-CGG | Gln-Arg | Atopy, |
| | | | | association |
| | | | | with |

Category 5-Generation and Transmission of Nervous Impulses
Prion protein

| | Total number of |
|---|---|
| Mutation type | mutations |
| Nucleotide substitutions (missense/nonsense) | 14 |
| Nucleotide substitutions (splicing) | 0 |
| Nucleotide substitutions (regulator) | 0 |
| Small deletions | 0 |
| Small insertions | 0 |
| Small indels | 0 |
| Gross deletions | 0 |
| Gross insertions & duplications | 0 |
| Complex rearrangements (including inversions) | 0 |
| Repeat variations | 0 |
| TOTAL | 14 |

| Accession | | | | |
|---|---|---|---|---|
| Number | Codon | Nucleotide | Amino acid | Phenotype |
| CM890102 | 102 | CCG-CTG | Pro-Leu | Gerstmann-Straeussler syndrome |
| CM930595 | 105 | CCA-CTA | Pro-Leu | Gerstmann-Straeussler syndrome |
| CM890103 | 117 | GCA-GTA | Ala-Val | Gerstmann-Straeussler syndrome |
| CM890104 | 129 | cATG-GTG | Met-Vat | Gerstmann-Straeussler syndrome |
| CM971202 | 171 | AAC-AGC | Asn-Ser | Schizophrenia |
| CM910305 | 178 | cGAC-AAC | Asp-Asn | Creutzfeld-Jakob syndrome |
| CM930596 | 180 | cGTC-ATC | Val-Ile | Creutzfeld-Jakob syndrome |
| CM971203 | 183 | cACA-GCA | Thr-Ala | Spongiform encephalopathy, familial |
| CM920588 | 198 | TTC-TCC | Phe-Ser | Gerstmann-Stracussler syndrome |
| CM890105 | 200 | cGAG-AAG | Glu-Lys | Creutzfeld-Jakob syndrome |
| CM961133 | 208 | CGC-CAC | Arg-His | Creutzfeld-Jakob syndrome |
| CM930597 | 210 | gGTT-ATT | Val-Ile | Creutzfeld-Jakob syndrome |
| CM920589 | 217 | CAG-CGG | Gln-Arg | Gerstmann-Straeusster syndrome |
| CM930598 | 232 | ATG-AGG | Met-Arg | Creutzfetd-Jakob syndrome |

Category 6-Growth and Differentiation
Vitamin D receptor

| Mutation type | Total number of mutations |
|---|---|
| Nucleotide substitutions (missense/nonsense) | 10 |
| Nucleotide substitutions (splicing) | 1 |
| Nucleotide substitutions (regulatory) | 0 |
| Small deletions | 0 |
| Small insertions | 0 |
| Small indels | 0 |
| Gross deletions | 0 |
| Gross insertions & duplications | 0 |
| Complex rearrangements (including inversions) | 0 |
| Repeat variations | 0 |
| TOTAL | 11 |

| Accession | | | | |
|---|---|---|---|---|
| Number | Codon | Nucleotide | Amino acid | Phenotype |
| CM971505 | 30 | cCGA-TGA | Arg-Term | Rickets, vitamin D resistant |
| CM880062 | 33 | GGC-GAC | Gly-Asp | Rickets, vitamin D resistant |
| CM961380 | 46 | GGC-GAC | Gly-Asp | Rickets, vitamin D resistant |
| CM910389 | 50 | CGA-CAA | Arg-Gln | Rickets, vitamin D resistant |
| CM880063 | 73 | CGA-CAA | Arg-Gln | Rickets, vitamin D resistant |
| CM900227 | 80 | CGG-CAG | Arg-Gln | Rickets, vitamin D resistant |
| CM930718 | 152 | cCAG-TAG | Gln-Term | Rickets, vitamin D resistant |
| CM930719 | 274 | CGC-CTC | Arg-Leu | Rickets, vitamin D resistant |
| CM890115 | 295 | TACc-TAA | Tyr-Term | Rickets, vitamin D resistant |
| CM971506 | 305 | CACa-CAG | His-Gln | Rickets, vitamin D resistant |

| Accession | | Donor/ | Relative | | |
|---|---|---|---|---|---|
| Number | IVS | Acceptor | location | Substitution | Phenotype |
| CS961654 | 4 | ds | +5 | G-C | Rickets, vitamin D resistant |

The identification of the core group of genes considered to have an important effect on the physiological and pathophysiological processes of disease enables attention to be focussed on ascertaining, identifying and cataloguing the genetic vatriation within the core group of genes utilising tried and tested technologies and techniques.

### EXAMPLE 6

Identifying and Detecting Polymorphic Variation in the Core List of Genes

The human genome is known to be highly variable in different individuals. Variation exists in approximately one nucleic acid residue in every 300. Although a single nucleic acid change (single nucleotide polymorphism, SNP e.g. Schafer and Hawkins 1997, Nickerson et al 1998, Rieder et al 1998, SNP Consortium 1999) is the commonest form of genetic variation, other more complex forms also occur for example:

| Type of variation | Example |
|---|---|
| Deletion | intronic deletion in the angiotensin |
| | converting enzyme gene |
| Insertion | 144 bp insertion in the prion gene |
| Repeats | Huntingtin gene in Huntington's chorea |

These more complex forms of genetic variations account for more than 40% of the genetic changes associated with human disease.

Variations in human gene sequences, which are present in more than 1% of the population, are known as polymorphisms. These changes in genetic sequence can be detected by a variety of methods, which allow the direct sequencing and correct alignment of nucleotides (e.g. the Sanger method). However, this method is prone to error and multiple runs are required to ensure accuracy. More recently (Schafer and Hawkins 1997, Gilles et al 1999) many other techniques have been developed to, accurately and sensitively, identify the presence of polymorphic variation based on:

restriction fragment length polymorphisms using Southern blots

allele specific extensions of a detection primer using high fidelity enzymes

scanning for single strand conformational polymorphisms

gel mobility detection of heteroduplexs

detection of denaturing gradient differences using gel electrophoresis

ribonuclease cleavage of RNA:RNA or RNA:DNA heteroduplexes

chemical cleavage of heteroduplex mismatches

gel based detection of resolvase cleavage using T4 endonuclease

radioactive labelling and multi-photon detection

detection of altered banding patterns on gels using cleavage fragment length polymorphisms

recognition of heteroduplex mismatches using E. Coli mismatch repair enzymes

DNA variation detection using denaturing high performance liquid chromatography

matrix assisted laser desorption/ionisation time of flight mass spectrometry

electronic array of DNA probes on silicon microchips

Therefore, given an identified gene sequence, the technology to identify polymorphic variation is well established and is generally applicable to any section of the human genome. (Nickerson et al 1998, Wang et al 1998, Rieder et al 1999).

In addition computational approaches can also be used to search for and assess polymorphic variation in existing gene sequence databases (as confirmed by Buetow et al 1999).

Thus the methods of generating the nucleotide sequence required for the design of an array or chip is well known to those skilled in the art.

However, for the purposes of an array design it would be useful to establish the frequency of a given polymorphism in the general population and thus derive a way of assessing its likely clinical importance. Polymorphisms are defined as being a genetic variation present in more than 1% of the population. In order to determine the frequency of a polymorphism in a given population a number of individual DNA samples will need to be investigated. The table below provides the number of DNA samples, which will need to be examined in order to determine the frequency of polymorphisms at a particular threshold of statistical certainty.

NUMBER OF DNA SAMPLES REQUIRED TO
DETECT POLYMORPHISMS

| Minimum Allele | | | Statistical |
|---|---|---|---|
| Frequency | Appears Once | Appears Twice | Certainty |
| >1% | 58 | 97 | 90% |
| | 75 | 119 | 95% |
| | 115 | 166 | 99% |
| >5% | 12 | 19 | 90% |
| | 15 | 24 | 95% |
| | 23 | 33 | 99% |
| >10% | 6 | 10 | 90% |
| | 8 | 12 | 95% |
| | 11 | 16 | 99% |

E.g. if a particular variant appears twice in 166 DNA samples, we can be 99% sure that the variant allele is present in >1% of the population.

The technologies and methodologies required for the identification and tabulation of polymorphic variation are of considerable value in the identification of genetic variation, which will be informative in the practice of medicine.

This invention provides a means of fusing the genomic and pharmacological profiles together with their clinical associations in such a way as to enhance and enable the provision of individually tailored therapeutic packages for enhanced healthcare management.

In addition, the use of such devices and the tabulating of genomic variations that lead to or predispose to disease, will lead to revolutionary insights into the pathophysiology of diseases. These may well lead to the classical definitions of disease states being sub-divided or re-organised into specific genomic configurations, creating the potential for new therapeutic approaches (as indicated in Drews and Ryser 1997).

The actual demonstration of associations between disease, outcomes, adverse events or specific symptom clusters will emerge as the result of clinical trials and investigations using accepted approaches and methods.

### EXAMPLE 7

### Analysis of Database to Ascertain Genotype/Phenotype Relationships

The generation of genetic profiling data and its analysis alongside clinical information derived from patients presents considerable challenges for data handling and analysis. The volume of information, number of information categories and the variable nature of the information (e.g. dimensional or categorical) ensure that the operation of a database combining genetic and clinical information to generate a prognostic outcome is a complex task.

However, the complexity can be dealt with using existing analytical approaches. Association analysis between genetic polymorphisms can be dealt with by using standard statistical techniques (analysis of variance, meta-analysis etc) with appropriate corrections for multiple testing. The thresholds for statistical significance will be derived from scientific convention (e.g. significance at the 5% level following Bonnferoni correction). The data concerning genotype/phenotype relationships between the core group of genes and clinical signs and symptoms and therapeutic interventions will form a central component of the database.

The creation of a database containing and elaborating on such genotype/phenotype relationships will become an important tool for the practice of molecular medicine and the development of healthcare management. In order to derive benefit from such a database it must be capable (following interrogation using a patients profile of genetic variation derived from the core group of genes) of analysing the profile and providing a meaningful output to the healthcare professional which will provide guidance on the prognosis, healthcare management and therapeutic interventions appropriate to the patient.

The generation of such an output can be achieved using machine learning algorithms. The genetic algorithm (Goldberg 1989, Fogarty and Ireson 1994) has been shown to provide a general process for achieving good results for search in large noisy domains. Starting from a population of randomly generated points in a search space, and given an evaluation of each of those points, the genetic algorithm is designed to converge the population to an optimum point in the search space. Processes of data selection, crossover, mutation and replacement of old members of the dataset achieve this with new members of more value. The effective use of the genetic algorithm process is a representation of the search space, which is responsive to the heuristics, embodied in the genetic operators.

The user must also supply an evaluation function identifying the degree to which the point in space approaches an optimum (‘weighting’) such that the selection operator for propagation through the dataset can choose them.

The genetic algorithm can be used to find predictively meaningful categories that is:

intervals of continuous attribute values

sets of nominal attribute values

combinations of attributes

Together these attributes can create a simple Bayesian classifier for aspects of healthcare management.

Additional techniques (e.g. Bahadur-Lazarsfeld expansion) enable second order approximation of dependencies between predictive attributes. This allows the full complexity of the individual's genetic variation profile and the specifics of their clinical, psychological and social state to be assessed in order to produce an output concerning their prognosis, healthcare management and the possibilities for therapeutic intervention.

Assembly of such data will allow the merging of accepted treatment algorithms with the polymorphic variation underlying specific aspects of genomic functionality. This will produce new algorithms that will provide a prognostic indication for individual patients and, coupled with the expertise of their responsible clinician, allow the appropriate healthcare decisions to be made in a pro-active way.

The identification of genetic variation in the core list of genes and its application to healthcare management will have significant beneficial effects on the way in which clinicians will be able to formulate plans for healthcare management.

This will be seen in at least two ways. The first by enabling the targeting of resources at appropriate individuals (see Example 8) and the second by enabling an objective risk assessment of the optimum configuration for different types of therapeutic intervention (e.g drugs, surgery, radiotherapy, occupational therapy) and the identification of those patients at significant risk of suffering adverse events from therapeutic intervention (see Example 9).

### EXAMPLE 8

### Clinical Management of Familial Adematous Polyposis

Familial adenomatous polyposis (FAP) is an autosomal dominant disorder which typically presents with colorectal cancer (CRC) in early adult life secondary to extensive adenomatous polyps of the colon. Polyps also develop in the upper gastrointestinal tract and malignancies may occur in other sites including the brain and the thyroid. Helpful diagnostic features include pigmented retinal lesions known as congenital hypertrophy of the retinal pigment, jaw cysts, sebaceous cysts, and osteomata. The APC gene at 5q21 is mutant in FAP.

Clinical Features

Familial adenomatous polyposis (FAP) is characterized by adenomatous polyps of the colon and rectum; in extreme cases the bowel is carpeted with a myriad of polyps. This is an aggressive premalignant disease with one or more polyps progressing through dysplasia to malignancy in untreated gene carriers with a median age at diagnosis of 40 years. Carcinoma may arise at any age from late childhood through the seventh decade. The presenting features are usually those of malignancy, such as weight loss and inanition, bowel obstruction, or bloody diarrhea. Cases of new mutation still present in these ways but in areas with well organized registers most other gene carriers are detected by bowel examination while still asymptomatic. Occasionally, the extracolonic features of the condition lead to presentation.

Petersen et al. (1993) demonstrated the feasibility of presymptomatic direct detection of APC mutations in each of 4 families. No change in the conventional FAP colon screening regimen was recommended for children found to have a mutation. In contrast, when direct tests indicated that an individual did not have the mutation, they recommended that screening be decreased. Three of the mutations were nonsense mutations and one was a frameshift mutation due to insertion of 1 nucleotide. In an evaluation of molecular genetic diagnosis in the management of familial polyposis, Maher et al. (1993) concluded that intragenic and closely linked DNA markers are informative in most families and that, in addition to the clinical benefits of presymptomatic diagnosis, the reduction in screening for low-risk relatives means that molecular genetic diagnosis is a cost-effective procedure.

Davies et al. (1995) found that families with mutations 3-prime of codon 1444 had significantly more lesions on dental panoramic radiographs (P less than 0.001) and appeared to have a higher incidence of desmoid tumors than did families with mutations at the 5-prime end. All 7 families except one with mutations 5-prime of exon 9 did not express CHRPE. All of 38 individuals from 16 families with mutations between exon 9 and codon 1444 expressed CHRPE. The 11 individuals from 4 families with mutations 3-prime of codon 1444 did not express CHRPE. These results suggested that the severity of some of the features of Gardner syndrome may correlate with genotype in FAP.

Since an alteration of the APC gene occurs early in most colorectal tumors, detection of APC mutations in fecal tumor DNA could be a powerful tool for the diagnosis of noninvasive cancer. Deuter and Muller (1998) described a highly sensitive and nonradioactive heteroduplex-PCR method (HD-PCR) for detecting APC mutations in stool DNA.

Petersen et al. (1989) demonstrated how one could use linkage information to modify the standard recommendations for follow-up. For example, in the family of an affected 36-year-old man with a positive family history of APC, there were 4 asymptomatic children under the age of 10 years. Before linkage analysis, all children had a 50% risk. Screening protocols would call for annual sigmoiloscopy in all beginning at age 12 years. With the linkage information, one could state to the family with 98% confidence that 3 of the children did not inherit the gene and that 1 child did. That child could be screened annually; the others would have screening every 3 years beginning at ages 12 or 13 and continuing until age 35.

### EXAMPLE 9

### Genetic Variation in Drug Targets and Drug Metabolizing Enzymes

Therapeutic intervention by the use of drugs is a common mode of clinical treatment. However, this is not without difficulty (Weatherall, Leadingham and Warell 1996) and even hazard (Lazarou et al 1998). Drugs interact with the body in many different ways to produce their effect. Some drugs act as false substrates of inhibitors for transport systems (e.g. calcium channels) or enzymes (acetylcholinesterase). Most drugs however, produce their effects by acting on receptors, usually located in the cell membrane, which normally respond to endogenous chemicals in the body (Weatherall, Leadingham and Warrell 1996). Drugs that activate receptors and produce a response are called agonists (e.g cholinomimetics). Antagonists combine with receptors but do not activate them, thus reduceing the probability of the transmitter substance combining with the receptor and so blocking receptor activation. The ability of the drug to interact with the receptor depends on the specificity of the drug for the receptor or ‘target’ (Brody, Lamer and Minneman 1998).

In addition to the main categories of agonist and antagonist, drugs also have mechanisms of action whereupon they interact with specific types of molecules—targets'—that include:

blockade of uptake or transport sites (e.g selective serotonin reuptake inhibitors)

enzyme inhibition (e.g. angiotensin convertying enzyme inhibitors, acetylcholinesterase inhibitors)

blockade of ion channels (calcium channel antagonists, anaesthetics)

However, many drugs are known to vary in their efficacy and side effects from patient to patient. This variation in drug response will be associated with the polymorphic variation in the drug target.

CNS MARKETED DRUGS

| Drug | Drug Target | Polymorphic? |
|---|---|---|
| Tricyclic antidepressants | Neurotransmitter (NA/5-HT) re- | ✓ |
| (TCA) | uptake proteins (NET & SERT) | |
| SSRIs | Selective serotonin transport re-uptake | ✓ |
| | protein (SERT) | |
| MAOIs | Monoamine oxidase A & B | ✓ |
| Benzodiazepines (GABA | GABA receptors | ✓ |
| facilitators)/GABA | | |
| antagonists. Barbiturates. | | |
| Beta-blockers | Noradrenaline (beta-adrenergic) | ✓ |
| | receptors | |
| Atypical antidepressants | Alpha-adrenoceptors | ✓ |
| Beta-adrenoceptors | Beta-adrenoceptors | |
| antagonists | | |
| Dopamine blockers/boosters | Dopamine receptors | ✓ |
| Dopamine blockers/ | Dopamine transporter (DAT1) | ✓ |
| boosters/depleters | | |
| Anticholinergics (muscarinic | Muscarinic receptors | ✓ |
| antagonists) | | |
| Anticholinergics | Nicotinic receptors | ✓ |
| (nicotinic antagonists) | | |
| Anticholinesterases | Acetylcholinesterase (ACHE) | ✓ |
| COMT inbibitor | Catechol-O-methyltransferase | ✓ |
| | (COMT) | |
| Sodium channel blocker | Sodium channel | ✓ |
| Opioid analgesics & | Opioid receptors (OPRM1; OPRK1; | ✓ |
| antagonists | OPRD 1) | |
| Antipsychotics/neuroleptics | 5-HT/D2 receptors | ✓ |
| (5-HT/D2 antagonists) | | |
| Antiinflammatory drugs | Cyclooxygenase (COX1, COX2) | ✓ |
| Antihistamines | Histamine receptors | ✓ |

CARDIOVASCULAR MARKETED DRUGS

| Drug | Drug Target | Polymorphic? |
|---|---|---|
| ACE inhibitors | Angiotensin converting enzyme (ACE) | ✓ |
| HMG CoA reductase | HMG CoA reductase | ✓ |
| inhibitors, e.g simvastatin | | |
| Angiotensin II antagonists | Angiotensinogen | ✓ |
| Calcium channel blocker | Calcium channel | ✓ |
| Thromboxane A2 synthase | Thromboxane A2 synthase | ✓ |
| inhibitor | | |
| A2 receptor antagonist | Thromboxane A2 receptor | ✓ |
| Potassium channel blocker | Potassium channel | ✓ |
| Na—H ion exchange (NHE) | Na—H ion exchanger (NHE) | ✓ |
| inhibitor | | |
| bile acid transport inhibitor | SLC1OA1 (sodium/bile acid cotransporter) | ✓ |
| bile acid transport inhibitor | SLCIOA2 (sodium/bile acid cotransporter) | ✓ |
| platelet aggregation inhibitor | Von Willebrand factor | ✓ |
| ACAT inhibitor | Acetoacetyl-CoA-thiolase (ACAT) | ✓ |
| Endothelin antagonist | Endothelin (EDN3) | ✓ |

GASTROINTESTINAL (Peptic ulcer) MARKETED DRUGS

| Drug | Drug Target | Polymorphic? |
|---|---|---|
| Proton pump inhibitor (e.g | H+/K+ adenosine triphosphatase (ATPase) | ✓ |
| omeprazole). | enzyme system (‘proton pump’) | |
| H2 antagonists | Histamine H2-rcceptor | ✓ |
| (e.g. cimetidine) | | |
| Muscarinic antagonists | Muscarinic m1 & m3 receptors | ✓ |
| (e.g. pirenepine) | | |
| Prostaglandins (inhibit | Adenylate cyclase, histamine-induced | ✓ |
| cAMP) | activity | |

Another problem the medical practitioner faces, is that certain patients may be particularly susceptible to drug addiction. Examples of drugs with known addictive properties are Amphetamines, Temazepam and Phenobarbitone, although having approved medicinal use e.g. phenobarbitone for epilepsy, they may cause problems of dependency and misuse in individuals. Knowledge of such an individual's susceptibility before prescribing certain drugs would be an advantage to the medical practitioner.

Any drug may produce unwanted or unexpected adverse events, these can range from trivial (slight nausea) to fatal (aplastic anaemia). One of the main reasons for adverse events following drug intake is the drug binding to a non specific or non target receptors in the body (Brody, Larner and Minneman 1998). Another reason is the interaction of the drug with other drugs given to the patient. This is a particular problem in the elderly who frequently suffer from multiple illnesses requiring many different classes of drugs and providing a real potential for drug interactions (Weatherall, Leadingham and Warrell 1996). The drug may also produce adverse events over time as the drug is absorbed, distributed, metabolised and excreted e.g. products of metabolising the drug may be reactive themselves and be toxic to the body. Being able to predict the likelihood of a particular individual suffering from an adverse event and the severity of that event would be an important tool for the practitioner. Many of the important components of the biological pathways involved in drug metabolism are coded by genes containing polymorphic variation.

METABOLISING ENZYMES

| Drug | Drug-metabolising enzyme | Polymorphic? |
|---|---|---|
| Most | Cytochromc P450 enzyme, CYP2C19 | ✓ |
| Most | Cytochrome P450 enzyme, CYP2D6 | ✓ |
| Most | UDP-glucuronosyltransferase | ✓ |
| Most | N-acetyltransferase (NAT 1) | ✓ |
| Most | Methyltransferase | ✓ |
| Most | Sulphotransferase | ✓ |
| Most | NADPH-cytochrome p450 reductase | ✓ |

The inventory of drugs and preparations both registered and in development which can be matched to drug targets exhibiting genetic polymorphisms can be found in standard works of reference, in particular the British National Formulary, 1998, the Dental Practioners' Formulary, 1998, Martindale, 1998, Herbal medicines, 1998. Drugs available in the United States can be found in U.S. Pharmacopeia, 1998, and drugs available in Japan can be found in Iryoyaku Nihon lyakuhinshu, 1998, Ippanyaku Nihon lyakuhinshu, 1998 and Hokenyaku Jiten, 1998. Drugs available in other countries can be found in the appropriate National Formularies. A list of drugs currently under development worldwide can be found in current journals and text (Pipeline pulse, 1999, Scrip, 1998, IDrugs, 1998, Current Opinion in Drug Discovery and Development, 1998).

The use of the Genostic approach described above would be of considerable utility in determining the likelihood and magnitude of therapeutic response to drugs in the inventories described above. Such difficulties can arise from adverse events, variations in metabolism and drug-drug interactions in situations where several diseases, requiring treatment, exist in a given patient. The potential for adverse events or deleterious outcomes could be ascertained in individuals, patients or populations in relation to all of the drugs referred to above. These factors are of considerable importance in enabling the selection and monitoring of therapeutic interventions and effective healthcare management.

Core Genes for Design and Manufacture of ‘Genostics’

We have elaborated on the value and utility to be derived from the gathering together of the genes which form the core gene list for the Genostic system.

These genes are elaborated below:

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

[image]

The core list of genes provides a platform for the design and application of profiling technologies to healthcare management. We have termed these designs for profiting “GenosticsTM”—an amalgam of genomics and prognosis.

This “GenosticTM” profiling of patients and persons will radically enhance the ability of clinicians, healthcare professionals and other parties to plan and manage healthcare provision and the targeting of appropriate healthcare resources to those deemed most in need.

The use of our invention could also lead to a host of new applications for such profiling technologies, such as identification of persons with particular work or environment related risk, selection of applicants for employment, training or specific opportunities or for the enhancing the planning and organisation of health services, education services and social services.

### References

Brenman, J. E.; Chao, D. S.; Xia, H.; Aldape, K.; Bredt, D. S. Nitric oxide synthetase complexed with dystrophin and absent from skeletal muscle sarcolemma in Duchenne muscular dystrophy. Cell 82: 743-752, 1995.

British National Formulary Number 35. British Medical Association and Royal Pharmaceutical Society of Great Britain (March 1998).

Brody T. M., Lamer J., Minneman K. P. Human Pharmacology Molecular to Clinical. 3^{rd} Ed. Mosby, 1998.

Buetow K H, Edmonson M N and Cassidy A B. Reliable identification of large numbers of candidate SNP's from public EST data. Nature Genetics 21, 323-5, 1999.

Cowen, S. G., Pipeline pulse, March 1999

Crooke S T. 1998. Optimising the impact of genomics on drug discovery and development. Nature Biotechnology 16 (Supplement): 29-30.

Davies, D. R.; Armstrong, J. G.; Thakker, N.; Homer, K.; Guy, S. P.; Clancy, T.; Sloan, P.; Blair, V.; Dodd, C.; Wames, T. W.; Harris, R.; Evan, D. G. R.: Severe Gardner syndrome in families with mutations restricted to a specific region of the APC gene. Am. J. Hum. Genet. 57: 1151-1158, 1995.

Dental Practitioners' Formulary, 1998-2000 Edition. British Medical Association and Royal Pharmaceutical Society of Great Britain (1998).

Deuter, R.; Muller, O.: Detection of APC mutations in stool DNA of patients with colorectal cancer by HD-PCR. Hum. Mutat. 11: 84-89, 1998. Drew S (1998). HRT—More good than harm. Geriatric Medicine 28: 23-26.

Drmanac S, Kita D, Labat I, Hauser B, Schmidt C, Burczak J D and Drmanac R. 1998. Accurate sequencing by hybridisation for DNA diagnostics and individual genomics. Nature Biotechnology 16:54-58.

Drews. J. 1997. Nature Biotechnology 14: 1516-1518.

Drews J and Ryser S. 1997. Nature Biotechnology 15: 1318-1319.

Fogarty T. C. and Ireson N. S. (1994) Evolving Bayseian classifiers for credit control—a comparison with other machine-learning methods. IMA Journal of Mathematics Applied in Business and Industry 5, 63-75.

Fox S 1998. Genomic Diagnostics: the next stage in drug development.

Gilles P N. 1999. Single nucleotide polymorphic discrimination by an electronic dot blot assay on semiconductor microchips. Nature Biotechnology 17 (4): 365-370.

Goldberg D. E. (1989) Genetic algorithms in search optimisation and machine learning. Addison-Wesley.

Goodman and Gillman. The Pharmacological Basis of Therapeutics, 9^{th} Ed. McGrawHill, New York 1996.

Griffin T J., Tang W and Smith L M. 1997. Genetic analysis by peptide nucleic acid affinity MALDI-TOF mass spectrometry. Nature Biotechnology 15 1368-1372.

Herbal Medicines, 1998

Lander E S. 1996. The new genomics: global views of biology. Science 274:540-545.

Lazarou J, Pomeranz B H, Corey P N. 1998. Incidence of adverse drug reactions in hospitalised patients: a meta-analysis of prospective studies. JAMA Apr 15; 279 (15): 1200-5

Marshall A. 1997. Getting the right drug into the right patient. Nature Biotechnology. 151249-1252.

Magee, T.; Fuentes, A. M.; Garban, H.; Rajavashisth, T.; Marquez, D.; Rodriguez, J.A.; Rajfer, J.; Gonzalez-Cadavid, N. F. Cloning of a novel neuronal nitric oxide synthase expressed in penis and lower urinary trac Biochem. Biophys. Res. Commun 226: 145-151, 1996.

Maher, E. R.; Barton, D. E.; Slatter, R.; Koch, D. J.; Jones, M. H.; Nagase, H.; Payne, S. J.; Charles, S. J.; Moore, A. T.; Nakamura, Y.; Ferguson-Smith, M. A.: Evaluation of molecular genetic diagnosis in the management of familial adenomatous polyposis coli: a population based study. J. Med. Genet. 30: 675-678, 1993.

Marshall A and Hodgson J 1998. DNA chips: an array of possibilities. Nature Biotechnology 16:27-31.

Martindale, 1998. Royal Pharmaceutical Society of Great Britain.

Nelson, R. J.; Demas, G. E.; Huang, P. L.; Fishman, M. C.; Dawson, V. L.; Dawson, T. M.; Snyder, S. H.: Behavioural abnormalities in male mice lacking neuronal nitric oxide synthase. Nature 378: 383-386, 1995.

Nickerson D A, et al DNA sequence diversity in a 9.7-kb region of the human lipoprotein lipase gene. Nature Genetics 19, 233-40, 1998.

Pathare S R and Paton C. ABC of mental health—psychotropic drug treatment. British Medical Journal 315: 661-664, 1997.

Petersen, G. M.; Francomano, C.; Kinzler, K.; Nakamure, Y.: Presymptomatic direct detection of adenomatous polyposis coli (APC) gene mutations in familial adenomatous polyposis. Hum. Genet. 91: 307-311, 1993.

Patersen, G. M.; Shohat, T.; Brown, J.; Nakamura, Y.: Genetic counseling for familial adenomatous polyposis (FAP) with chromosome 5q linkage information. (Abstract) Am. J. Hum. Genet. 45 (suppl.): A125 only, 1989. Poste G. 1998. Molecular medicine and information-based targetted of healthcare. Nature Biotechnology. 16 (Supplement): 19-21.

Rieder M J, Taylor S L, Clark A G and Neckerson). Sequence variation in the human angiotensin converting enzyme. Nature Genetics 22, 62-9, 1999.

Schafer A J and Hawkins J R 1997. DNA variation and the future of human genetics. Nature Biotechnology 16: 33-44.

Taylor D. and Kerwin R. (1997). Clozapine's role in the treatment of schizophrenia. Prescriber—Suplement.

Tyagi s, Bratu D P and Kramer F R 1998. Multicolour molecular beacons for allele discrimination. Nature Biotechology 16: 49-53.

U.S. Pharmacopeia, 1998.

Wang D G, et al. Large-scale identification, mapping andgenotyping of single nucleotide polymorphisms in the human genome. Science 265, 2049-54, 1998.

Weatherall D j, Ledingham J G G and Warrel D A. Eds Oxford Textbook of Medicine 3r^{d} Edition. Oxford Medical Publications 1996.

Xie, J.; Roddy, P.; Rife, T. K.; Murad, F.; Young, A. P.: Two closely linked but separable promoters for human neuronal nitric oxide synthase gene transcription Proc. Nat. Acad. Sci 92: 1242-1246, 1995.

1. A set of nucleotide probes for detecting relevant variants (mutations and polymorphisms), e.g. nucleotide substitutions (missense, nonsense, splicing and regulatory), small deletions, small insertions, small insertion deletions, gross insertions, gross deletions, duplications, complex rearrangements and repeat variations in a target group of genes; said probes being complementary to DNA and RNA sequences of said group of genes; characterised in that said group is a core group of genes consisting of substantially all of the following:

| | HUGO GENE | PROTEIN |
|---|---|---|
| CORE GENE LIST | SYMBOL | FUNCTION |
| 11beta hydroxysteroid dehydrogenase 2 | HSD11B2 | E |
| 17beta hydroxysteroid dehydrogenase 1 | HSD17B1 | E |
| 17beta hydroxysteroid dehydrogenase 3 | HSD17B3 | E |
| 17beta hydroxysteroid dehydrogenase 4 | HSD17B4 | E |
| 17beta hydroxysteroid oxidoreductase | | E |
| 18-hydroxysteroid oxidoreductase | | E |
| 2,3-bisphosphoglycerate mutase | BPGM | E |
| 2,4-dienoyl CoA reductase | DECR | E |
| 3 beta hydroxysteroid dehydrogenase 2 | HSD3B2 | E |
| 3-oxoacid CoA transferase | OXCT | E |
| 4-hydroxyphenylpyruvate dioxygenase | HPD | E |
| 5,10-methylenetetrahydrofolate reductase | MTHFR | E |
| (NADPH) | | |
| 5-adenosyl homocysteine hydrolase | | E |
| 6-phosphofructo-2-kinase | PFKFB1 | E |
| 6-pyruvoyltetrahydropterin synthase | PTS | E |
| Acetoacetyl 1-CoA-thiolase | ACAT1 | E |
| Acetoacetyl 2-CoA-thiolase | ACAT2 | E |
| Acetyl CoA acyltransferase | ACAA | E |
| Acetyl CoA carboxylase | ACC | E |
| Acetyl CoA carboxylase alpha | ACACA | E |
| Acetyl CoA synthase | | E |
| Acetylcholinesterase | ACHE | E |
| Acid phosphatase 2, lysosomal | ACP2 | E |
| Aconitase | | E |
| Acyl CoA dehydrogenase, long chain | ACADL | E |
| Acyl CoA dehydrogenase, medium chain | ACADM | E |
| Acyl CoA dehydrogenase, short chain | ACADS | E |
| Acyl CoA dehydrogenase, very long chain | ACADVL | E |
| Acyl CoA synthetase, long chain, 1 | LACS1 | E |
| Acyl CoA synthetase, long chain, 2 | LACS2 | E |
| Acyl CoA synthetase, long chain, 4 | ACS4 | E |
| Acyl malonyl condensing enzyme | | E |
| Acyl-CoA thioesterase | | E |
| ADAM (A disintegrin and | ADAM1 | E |
| metalloproteinase) 1 | | |
| ADAM (A disintegrin and | ADAM10 | E |
| metalloproteinase) 10 | | |
| ADAM (A disintegrin and | ADAM11 | E |
| metalloproteinase) 11 | | |
| ADAM (A disintegrin and | ADAM12 | E |
| metalloproteinase) 12 | | |
| ADAM (A disintegrin and | ADAM13 | E |
| metalloproteinase) 13 | | |
| ADAM (A disintegrin and | ADAM14 | E |
| metalloproteinase) 14 | | |
| ADAM (A disintegrin and | ADAM15 | E |
| metalloproteinase) 15 | | |
| ADAM (A disintegrin and | ADAM16 | E |
| metalloproteinase) 16 | | |
| ADAM (A disintegrin and | ADAM17 | E |
| metalloproteinase) 17 | | |
| ADAM (A disintegrin and | ADAM18 | E |
| metalloproteinase) 18 | | |
| ADAM (A disintegrin and | ADAM19 | E |
| metalloproteinase) 19 | | |
| ADAM (A disintegrin and | ADAM2 | E |
| metalloproteinase) 2 | | |
| ADAM (A disintegrin and | ADAM3A | E |
| metalloproteinase) 3A | | |
| ADAM (A disintegrin and | ADAM3B | E |
| metalloproteinase) 3B | | |
| ADAM (A disintegrin and | ADAM4 | E |
| metalloproteinase) 4 | | |
| ADAM (A disintegrin and | ADAM5 | E |
| metalloproteinase) 5 | | |
| ADAM (A disintegrin and | ADAM6 | E |
| metalloproteinase) 6 | | |
| ADAM (A disintegrin and | ADAM7 | E |
| metalloproteinase) 7 | | |
| ADAM (A disintegrin and | ADAM8 | E |
| metalloproteinase) 8 | | |
| ADAM (A disintegrin and | ADAM9 | E |
| metalloproteinase) 9 | | |
| Adenosine deaminase | ADA | E |
| Adenosine monophosphate deaminase | AMPD | E |
| Adenylate cyclase 1 | ADCY1 | E |
| Adenylate cyclase 2 | ADCY2 | E |
| Adenylate cyclase 3 | ADCY3 | E |
| Adenylate cyclase 4 | ADCY4 | E |
| Adenylate cyclase 5 | ADCY5 | E |
| Adenylate cyclase 6 | ADCY6 | E |
| Adenylate cyclase 7 | ADCY7 | E |
| Adenylate cyclase 8 | ADCY8 | E |
| Adenylate cyclase 9 | ADCY9 | E |
| Adenylate kinase | AK1 | E |
| Adenylate transferase | | E |
| Adenylosuccinate lyase | ADSL | E |
| ADP-ribosyltransferase | ADPRT | E |
| Adrenoleukodystrophy gene | ALD | E |
| Alanine-glyoxylate aminotransferase | AGXT | E |
| Alcohol dehydrogenase 1 | ADH1 | E |
| Alcohol dehydrogenase 2 | ADH2 | E |
| Alcohol dehydrogenase 3 | ADH3 | E |
| Alcohol dehydrogenase 4 | ADH4 | E |
| Alcohol dehydrogenase 5 | ADH5 | E |
| Alcohol dehydrogenase 6 | ADH6 | E |
| Alcohol dehydrogenase 7 | ADH7 | E |
| Aldehyde dehydrogenase 1 | ALDH1 | E |
| Aldehyde dehydrogenase 10 | ALDH10 | E |
| Aldehyde dehydrogenase 2 | ALDH2 | E |
| Aldehyde dehydrogenase 5 | ALDH5 | E |
| Aldehyde dehydrogenase 6 | ALDH6 | E |
| Aldehyde dehydrogenase 7 | ALDH7 | E |
| Aldolase A | ALDOA | E |
| Aldolase B | ALDOB | E |
| Aldolase C | ALDOC | E |
| Alkylglycerone phosphate synthase | AGPS | E |
| alpha1-antichymotrypsin | AACT | E |
| alphal-antitrypsin | PI | E |
| alpha2-antiplasmin | PLI | E |
| alpha-amino adipic semialdehyde synthase | | E |
| alpha-amylase | | E |
| alpha-dextrinase | | E |
| alpha-Galactosidase A | GLA | E |
| Alpha-galactosidase B, GALB | NAGA | E |
| alpha-glucosidase, neutral C | GANC | E |
| alpha-glucosidase, neutral AB | GANAB | E |
| Peptidylglycine alpha-amidating | PAM | E |
| monooxygenase | | |
| alpha-ketoglutarate dehydrogenase | | E |
| alpha-L-Iduronidase | IDUA | E |
| Aminomethyltransferase | AMT | E |
| Aminopeptidase P | XPNPEP2 | E |
| Amylo-1,6-glucosidase | AGL | E |
| Angiotensin converting enzyme | ACE, DCP1 | E |
| Angiotensinogen | AGT | E |
| Antithrombin III | AT3 | E |
| Apurinic endonuclease | APE | E |
| Arginase | ARG1 | E |
| Arginosuccinate lyase | ASL | E |
| Arginosuccinate synthetase | ASS | E |
| Arylsulfatase A | ARSA | E |
| Arylsulfatase B | ARSB | E |
| Arylsulfatase C | ARSC1 | E |
| Arylsulfatase D | ARSD | E |
| Arylsulfatase E | ARSE | E |
| Arylsulfatase F | ARSF | E |
| Asparagine synthetase | AS | E |
| Aspartate transcarbamoylase | | E |
| Aspartoacylase | ASPA | E |
| Aspartylglucosaminidase | AGA | E |
| ATP cobalamin adenoxyltransferase | | E |
| ATP sulphurylase | atpsk2 | E |
| ATP/ADP translocase | | E |
| beta-galactosidase | GLB1 | E |
| beta-glucosidase, neutral | | E |
| beta-Glucuronidase | GUSB | E |
| beta-ketoacyl reductase | | E |
| beta-N-acetylhexosaminidase, A | | E |
| beta-N-acetylhexosaminidase, B | | E |
| Bile acid coenzyme A: amino acid | BAAT | E |
| N-acyltransferase | | |
| Bile salt-stimulated lipase | CEL | E |
| Bilirubin UDP-glucuronosyltransferase | | E |
| Biotinidase | BTD | E |
| Bleomycin hydrolase | BLMH | E |
| Branched chain aminotransferase 1, | BCAT1 | E |
| cytosolic | | |
| Branched chain aminotransferase 2, | BCAT2 | E |
| mitochondrial | | |
| Branched chain keto acid | BCKDHA | E |
| dehydrogenase E1, alpha | | |
| polypeptide | | |
| Branched chain keto acid | BCKDHB | E |
| dehydrogenase E1, beta | | |
| polypeptide | | |
| Brush border guanylyl cyclase | | E |
| Butyrylcholinesterase | BCHE | E |
| C1 inhibitor | | E |
| C17-20 desmolase | | E |
| C3 convertase | | E |
| Calpain | CAPN, CAPN3 | E |
| Carbamoylphosphate synthetase 1 | CPS1 | E |
| Carbamoylphosphate synthetase 2 | CPS2 | E |
| Carbonic anhydrase, alpha | CA1 | E |
| Carbonic anhydrase, beta | CA2 | E |
| Carbonic anhydrase 3 | CA3 | E |
| Carbonic anhydrase 4 | CA4 | E |
| Carboxylesterase 1 | CES1 | E |
| Carboxypeptidase | CPN | E |
| Carnitine acetyltransferase | CRAT | E |
| Carnitine acylcarnitine translocase | CACT | E |
| Carnitine palmitoyltransferase I | CPT1A | E |
| Carnitine palmitoyltransferase II | CPT2 | E |
| Catechol-O-methyltransferase | COMT | E |
| Cathepsin B | | E |
| Cathepsin D | | E |
| Cathepsin E | | E |
| Cathepsin G | CTSG | E |
| Cathepsin H | | E |
| Cathepsin K | CTSK | E |
| Cathepsin L | | E |
| Cathepsin S | | E |
| Caveolin 3 | CAV3 | E |
| Ceruloplasmin precursor | CP | E |
| Chitotriosidase | chit | E |
| Cholesterol ester hydroxylase | | E |
| Choline acetyltransferase | CHAT | E |
| Chymase | CHY1 | |
| Chymotrypsinogen | | E |
| Citrate synthase | | E |
| CoA transferase | | E |
| Coenzyme Q (CoQ)/ubiquinone | | E |
| Collagenic-like tail subunit of asymmetric | COLQ | E |
| acetylcholinesterase | | |
| Complex I | | E |
| Complex II | | E |
| Complex III | | E |
| Complex III | | E |
| Complex V | MTATP6 | E |
| Coproporphyrinogen oxidase | CPO | E |
| Creatine kinase—B and m | CKBE | E |
| Cu2+ transporting ATPase alpha | ATP7A | E |
| polypetide | | |
| Cu2+ transporting ATPase beta | ATP7B | E |
| polypeptide | | |
| Cyclic nucleotide phosphodiesterase 1B | PDE1B | E |
| Cyclic nuclcotide phosphodiesterase 1B1 | PDE1B1 | E |
| Cyclic nucleotide phosphodiesterase 2A3 | PDE2A3 | E |
| Cyclic nucleotide phosphodiesterase 3A | PDE3A | E |
| Cyclic nucleotide phosphodiesterase 3B | PDE3B | E |
| Cyclic nucleotide phosphodiesterase 4A | PDE4A | E |
| Cyclic nucleotide phosphodiesterase 4C | PDE4C | E |
| Cyclic nucleotide phosphodiesterase 5A | PDE5A | E |
| Cyclic nucleotide phosphodiesterase 6A | PDE6A | E |
| Cyclic nucleotide phosphodiesterase 6B | PDE6B | E |
| Cyclic nucleotide phosphodiesterase 7 | PDE7 | E |
| Cyclic nucleotide phosphodiesterase 8 | PDE8 | E |
| Cyclic nucleotide phosphodiesterase 9A | PDE9A | E |
| Cyclooxygenase 1 | COX1 | E |
| Cyclooxygenase 2 | COX2 | E |
| CYP11A1 | CYP11A1 | E |
| CYP11B1 | CYP11B1 | E |
| CYP11B2 | CYP11B2 | E |
| CYP17 | CYP17 | E |
| CYP19 | CYP19 | E |
| CYP1A1 | CYP1A1 | E |
| CYP1A2 | CYP1A2 | E |
| CYP1B1 | CYP1B1 | E |
| CYP21 | CYP21 | E |
| CYP24 | CYP24 | E |
| CYP27 | CYP27 | E |
| CYP27B1 | PDDR | E |
| CYP2A1 | CYP2A1 | E |
| CYP2A13 | CYP2A13 | E |
| CYP2A3 | CYP2A3 | E |
| CYP2A6V2 | CYP2A6V2 | E |
| CYP2A7 | CYP2A7 | E |
| CYP2B6 | CYP2B6 | E |
| CYP2C18 | CYP2C18 | E |
| CYP2C19 | CYP2C19 | E |
| CYP2C8 | CYP2C8 | E |
| CYP2C9 | CYP2C9 | E |
| CYP2D6 | CYP2D6 | E |
| CYP2E1 | CYP2E1 | E |
| CYP2F1 | CYP2F1 | E |
| CYP2J2 | CYP2J2 | E |
| CYP3A3 | CYP3A3 | E |
| CYP3A4 | CYP3A4 | E |
| CYP3A5 | CYP3A5 | E |
| CYP3A7 | CYP3A7 | E |
| CYP4A11 | CYP4A11 | E |
| CYP4B1 | CYP4B1 | E |
| CYP4F2 | CYP4F2 | E |
| CYP4F3 | CYP4F3 | E |
| CYP51 | CYP51 | E |
| CYP5A1 | CYP5A1 | E |
| CYP7A | CYP7A | E |
| CYP8 | CYP8 | E |
| Cystathionase | CTH | E |
| Cystathione beta synthase | CBS | E |
| Cytidine deaminase | CDA | E |
| Cytidine-5-prime-triphosphate synthetase | CTPS | E |
| Cytochrome a | | E |
| Cytochrome b-245 alpha | CYBA | E |
| Cytochrome b-245 beta | CYBB | E |
| Cytochrome b-5 | CYB5 | E |
| Cytochrome c | | E |
| Cytochrome c oxidase, MTCO | | E |
| D-beta-hydroxybutyrate dehydrogenase | | E |
| Dehydratase | | E |
| Delta 4-5 alpha-reductase | | E |
| Delta 4-5 oxosteroid isomerase | | E |
| Delta aminolevulinate dehydratase | ALAD | E |
| Delta aminolevulinate synthase 1 | ALAS1 | E |
| Delta aminolevulinate synthase 2 | ALAS2 | E |
| Delta(4)-3-oxosteroid 5-beta reductase | | E |
| Delta-7-dehydrocholesterol reductase | DHCR7 | E |
| Deoxycorticosterone (DOC) receptor | | E |
| Deoxycytidine kinase DCK | | E |
| Deoxyuridine triphosphatase; dUTPase | | E |
| DHEA sulfotransferase | STD | E |
| Dihydrodiol dehydrogenase 1 | DDH1 | E |
| Dihydrofolate reductase | DHFR | E |
| Dihydrolipoyl dehydrogenase | | E |
| Dihydrolipoyl dehydrogenase 2 | PDHA | E |
| Dihydrolipoyl succinyltransferase | DLST | E |
| Dihydrolipoyl transacetylase | PDHA | E |
| Dihydroorotase | | E |
| Dihydropyramidinase | DPYS | E |
| Dihydroxyacetonephosphate | DHAPAT | E |
| acyltransferase | | |
| Dihyropyrimidine dehydrogenase | DPYD | E |
| DM-Kinase | DMPK | E |
| DNA directed polymerase, alpha | POLA | E |
| DNA glycosylases | | E |
| DNA helicases | | E |
| DNA Ligase 1 | LIG1 | E |
| DNA methyltransferase | DNMT | E |
| Methylguanine-DNA methyltransferase | MGMT | E |
| DNA polymerase 1 | | E |
| DNA polymerase 2 | | E |
| DNA polymerase 3 | | E |
| DNA primase | | E |
| DNA-dependent RNA polymerase | | E |
| DOPA decarboxylase | DDC | E |
| Dopamine beta hydroxylase | DBH | E |
| Dysferlin | DYS, DYSF | E |
| Dystrophia myotonica | DM, DMPK | E |
| Dystrophia myotonica, atypical | DM2 | E |
| Elastase 1 | ELAS1 | E |
| Elastase 2 | ELAS2 | E |
| Electron-transferring flavoprotein | ETFDH | E |
| dehydrogenase | | |
| Enolase | ENO1 | E |
| Enoyl CoA hydratase | | E |
| Enoyl CoA isomerase | | E |
| Enoyl CoA reductase | | E |
| Enterokinase | PRSS7, ENTK | E |
| Eosinophil peroxidase | EPX | E |
| Epilepsy, benign neonatal 4 gene | ICCA | E |
| Epilepsy, female restricted | EFMR | E |
| Epilepsy, progressive myoclonic 2 gene | EPM2A | E |
| Epoxide hydrolase 1, microsomal | EPHX1 | E |
| Excision repair complementation | ERCC1 | E |
| group 1 protein | | |
| Excision repair complementation | ERCC2 | E |
| group 2 protein | | |
| Excision repair complementation | ERCC3 | E |
| group 2 protein | | |
| Excision repair complementation | ERCC4 | E |
| group 4 protein | | |
| Excision repair complementation | ERCC6 | E |
| group 6 protein | | |
| FADH dehydrogenase | | E |
| Ferrochelatase | FECH | E |
| Flavin-containing monooxygenase 1 | FMO1 | E |
| Flavin-containing monooxygenase 2 | FMO2 | E |
| Flavin-containing monooxygenase 3 | FMO3 | E |
| Flavin-containing monooxygenase 4 | FMO4 | E |
| Formiminotransferase | | E |
| Fructose-1,6-diphosphatase | FBP1 | E |
| Fucosidase alpha-L-1 | FUCA1 | E |
| Fucosidase alpha-L-2 | | E |
| Fumarase | FH | E |
| Fumarylacetoacetase | FAH | E |
| GABA transaminase | ABAT | E |
| Gadd45 (growth arrest & | | E |
| DNA-damage-inducible protein) | | |
| Galactocerebrosidase | GALC | E |
| Galactokinase | GALK1 | E |
| Galactose 1-phosphate uridyl-transferase | GALT | E |
| Gastric Intrinsic factor, GIF | GIF | E |
| Glucokinase | GCK | E |
| Glucosaminyl (N-acetyl) transferase 2, | GCNT2 | E |
| I-branching enzyme | | |
| Glucose-6-phosphatase | G6PC | E |
| Glucose-6-phosphatase translocase | G6PT1 | E |
| Glucose-6-phosphate dehydrogenase | G6PD | E |
| Glucosidase, acid alpha | GAA | E |
| Glucosidase, acid beta | GBA | E |
| Glutamate decarboxylase, GAD | GAD1 | E |
| Glutamate dehydrogenase | GLUD1 | E |
| Glutamate-cysteine ligase | GLCLC | E |
| Glutamine phosphoribosylpyrophosphate | | E |
| amidotransferase/PRPP | | |
| amidotransferase | | |
| Glutamine synthase | | E |
| Glutaryl-CoA dehydrogenase | GCDH | E |
| Glutathione peroxidase, GPX1 | GPX1 | E |
| Glutathione peroxidase, GPX2 | GPX2 | E |
| Glutathione reductase, GSR | GSR | E |
| Glutathione S-transferase mu 1, GSTM1 | GSTM1 | E |
| Glutathione S-transferase mu 4, GSTM4 | | E |
| Glutathione S-transferase theta 1, GSTT1 | GSTT1 | E |
| Glutathione S-transferase theta 2, GSTT2 | | E |
| Glutathione S-transferase, GSTP1 | GSTP1 | E |
| Glutathione S-transferase, GSTZ1 | GSTZ1 | E |
| Glutathione synthetase | GSS | E |
| Glyceraldehyde-3-phosphate | GAPDH | E |
| dehydrogenase, GADPH | | |
| Glycerol kinase | GK | E |
| Glycerophosphate dehydrogenase 2 | GPD2 | E |
| Glycinamide ribonucleotide (GAR) | GART | E |
| transformylase | | |
| Glycine dehydrogenase | GLDC | E |
| Glycogen branching enzyme | GBE1 | E |
| Glycogen phosphorylase | PYGL | E |
| Glycogen synthase 1 (muscle) | GLYS1 | E |
| Glycogen synthase 2 (liver) | GYS2 | E |
| Glycosyltransferases, ABO blood group | ABO | E |
| GM2 ganglioside activator protein, GM2A | GM2A | E |
| Guanidinoacetate N-methyltransferase | GAMT | E |
| Guanylate cyclase 2D, membrane | GUCY2D | E |
| (retina-specific) | | |
| Guanylate cyclase activator 1A (retina) | GUCA1A | E |
| Guanylate kinase | | E |
| Guanylyl cyclase | | E |
| Hacme regulated inhibitor kinase | | E |
| Heparan sulfamidase | | E |
| Hepatic lipase | LIPC | E |
| Hepatic nuclear factor-3-beta | HNF3B | E |
| Hepatic nuclear factor-4-alpha | HNF4A | E |
| Hexokinase 1 | HK1 | E |
| Hexokinase 2 | HK2 | E |
| Hexosaminidase A | HEXA, TSD | E |
| Hexosaminidase B | HEXB | E |
| Histidase | | E |
| HMG-CoA lyase | HMGCL | E |
| HMG-CoA reductase | HMGCR | E |
| HMG-CoA synthase | HMGCS2 | E |
| Holocarboxylase synthetase | HLCS | E |
| Homogentisate 1,2 dioxygenase | HGD | E |
| Hormone-sensitive lipase | HSL | E |
| HSSB, replication protein | | E |
| Hydroxyacyl glutathione hydrolase | HAGH | E |
| Hypoxanthine-guanine | HPRT | E |
| phosphoribosyltransferase, HGPRT | | |
| Hypoxia inducible factor 1 | HIF1A | E |
| Hypoxia inducible factor 2 | | E |
| Ibonucleoside diphosphate reductase | | E |
| Iduronate 2 sulphatase | IDS | E |
| Inosine monophosphate dehydrogenase, | | E |
| IMPDH | | |
| Inosine triphosphatase | ITPA | E |
| Inter-alpha-trypsin inhibitor, IAT1 | | E |
| Iodothyronine-5′-deiodinase, type 1 and 2 | | E |
| IP3 kinase | | E |
| Isocitrate dehydrogenase | | E |
| Isovaleric acid CoA dehydrogenase | IVD | E |
| Ketohexokinase | KHK | E |
| ketolase | | E |
| Kynurenine hydroxylase | | E |
| Kynureninease | | E |
| Lactase | | E |
| Lactate dehydrogenase, A | LDHA | E |
| Lactate dehydrogenase, B | LDHB | E |
| Lecithin-cholesterol acyltransferase | LCAT | E |
| Leukotriene A4 synthase | LTA4S | E |
| Leukotriene B4 synthase | LTB4S | E |
| Leukotriene C4 synthase | LTC4S | E |
| Lipoamide dehydrogenase | OGDH | E |
| Lipoxygenase | | E |
| Lowe oculocerbrorenal syndrome gene | OCRL | E |
| Lysosomal acid lipase | LIPA | E |
| Lysyl hydroxylase | PLOD | E |
| Lysyl oxidase | LOX | E |
| Malate dehydrogenase, mitochondrial | MDH2 | E |
| Malonyl CoA decarboxylase | | E |
| Malonyl CoA transferase | | E |
| Maltase-glucoamylase | | E |
| Mannosidase, alpha B lysosomal | MANB | E |
| Mannosidase, beta A lysosomal | MANBA | E |
| Matrix metalloproteinase 1 | MMP1 | E |
| Matrix metalloproteinase 10 | MMP10 | E |
| Matrix metalloproteinase 11 | MMP11 | E |
| Matrix metalloproteinase 12 | MM12 | E |
| Matrix metalloproteinase 13 | MMP13 | E |
| Matrix metalloproteinase 14 | MMP14 | E |
| Matrix mctalloproteinase 15 | MMP15 | E |
| Matrix metalloproteinase 16 | MMP16 | E |
| Matrix metalloproteinase 17 | MMP17 | E |
| Matrix metalloproteinase 18 | MMP18 | E |
| Matrix metalloproteinase 19 | MMP19 | E |
| Matrix metalloproteinase 2 | MMP2 | E |
| Matrix metalloproteinase 3 | MMP3, | E |
| | STMY1 | |
| Matrix metalloproteinase 4 | MMP4 | E |
| Matrix metalloproteinase 5 | MMP5 | E |
| Matrix metalloproteinase 6 | MMP6 | E |
| Matrix metalloproteinase 7 | MMP7 | E |
| Matrix metalloproteinase 8 | MMP8 | E |
| Matrix metalloproteinase 9 | MMP9 | E |
| MEK kinase, MEKK | | E |
| Methionine adenosyltransferase | MAT1A, | E |
| | MAT2A | |
| Methionine synthasc | MTR | E |
| Methionine synthase reductase | MTRR | E |
| Methylmalonyl-CoA mutase | MUT | E |
| Mevalonate kinase | MVK | E |
| Mitochondrial trifunctional protein, alpha | HADHA | E |
| Mitochondrial trifunctional protein, | HADHB | E |
| beta subunit | | |
| Molybdenum cofactor synthesis 1 | MOCS1 | E |
| Molybdenum cofactor synthesis 2 | MOCS2 | E |
| Monoamine oxidase A | MAOA | E |
| Monoamine oxidase B | MAOB | E |
| Mucolipidoses | GNPTA | E |
| Muscle phosphorylase | PYGM | E |
| N-acetylgalactosamine-6-sulfate sulfatase | GALNS | E |
| N-acetylglucosamine-6-sulfatase | GNS | E |
| N-acetylglucosaminidase, alpha | NAGLU | E |
| N-acetyltransferase 1 | NAT1 | E |
| N-acetyltransferase 2 | NAT2 | E |
| NADH dehydrogenase | | E |
| NADH dehydrogenase (ubiquinone) Fe-S | NDUFS1 | E |
| NADH dehydrogenase (ubiquinone) Fe-S | NDUFS4 | E |
| NADH dehydrogenase (ubiquinone) | NDUFV1 | E |
| NADH-cytochrome b5 reductase | DIA1 | E |
| NADPH-dependent cytochrome | POR | E |
| P450 reductase | | |
| Neuroendocrine convertase 1 | NEC1, PCSK1 | E |
| Neutral endopeptidase | | E |
| Nitric oxide synthase 1, NOS1 | NOS1 | E |
| Nitric oxide synthase 2, NOS2 | NOS2 | E |
| Nitric oxide synthase 3, NOS3 | NOS3 | E |
| Nucleoside diphosphate kinase-A | NDPKA | E |
| Ornithine delta-aminotransferase | OAT | E |
| Ornithine transcarbamoylase | OTC, NME1 | E |
| Pancreatic amylase | | E |
| Pancreatic lipase | PNLIP | E |
| Pancreatic lipase related protein 1 | PLRP1 | E |
| Pancreatic lipase related protein 2 | PLRP2 | E |
| Paraoxonase PON1 | PON1 | E |
| Paraoxonase PON2 | PON2 | E |
| Paraoxonase PON3 | | E |
| PCNA (proliferating cell nuclear antigen) | | E |
| Pepsinogen | | E |
| Peroxidase, salivary | SAPX | E |
| Phenylalanine hydroxylase | PAH | E |
| Phenylalanine monooxygenase | | E |
| Phenylethanolamine N-methyltransferase, | PNMT | E |
| Phosphoenolpyruvate carboxykinase | PCK1 | E |
| Phosphofructokinase, liver | PFKL | E |
| Phosphofructokinase, muscle | PFKM | E |
| Phosphoglucomutase | | E |
| Phosphoglucose isomerase | GPI | E |
| Phosphoglycerate kinase 1 | PGK1 | E |
| Phosphoglycerate mutase 2 | PGAM2 | E |
| Phosphoribosyl pyrophosphate synthetase | PRPS1 | E |
| Phosphorylase kinase deficiency, liver | PHK | E |
| Phosphorylase kinase, alpha 1 (muscle) | PHKA1 | E |
| Phosphorylase kinase, alpha 2 | PHKA2 | E |
| Phosphorylase kinase, beta | PHKB | E |
| Phosphorylase kinase, delta | | E |
| Phosphorylase kinase, gamma 2 | PHKG2 | E |
| Pineolytic beta-receptors | | E |
| Plasminogen | PLG | E |
| Plasminogen activator inhibitor 1 | PAI1 | E |
| Plasminogen activator inhibitor 2 | PAI2 | E |
| Plasminogen activator receptor, Urokinase | UPAR; | S |
| | PLAUR | |
| Plasminogen activator, Tissue | PLAT; TPA | E |
| Plasminogen activator, Urokinase | UPA; PLAU | E |
| Poly (ADP-ribose) synthetase | PARS | E |
| Porphobilinogen deaminase | HMBS | E |
| Procollagen N-protease | | E |
| Procollagen peptidase | | E |
| Proline dehydrogenase | PRODH | E |
| Prolyl-4-hydroxylase | | E |
| Propionyl-CoA carboxylase, alpha | PCCA | E |
| Propionyl-CoA carboxylase, beta | PCCB | E |
| Prostasin, PRSS8 | PRSS8 | E |
| Protease nexin 2 | PN2 | E |
| Protective protein for beta-galactosidase | PPGB | E |
| Protein kinase A | | E |
| Protein kinase B | PRKB | |
| Protein kinase C, alpha | PRKCA | E |
| Protein kinase C, gamma | PRKCG | E |
| Protein kinase DNA-activated | PRKDC | E |
| Protein kinase G | | E |
| Protein phosphatase 1, regulatory | PPP1R3 | E |
| (inhibitor) | | |
| Protein phosphatase 2, regulatory | PPP2R1B | E |
| subunit A, isoform | | |
| Protoporphyrinogen oxidase | PPOX | E |
| Pterin-4-alpha-carbinolamiine | PCBD | |
| Purine nucleoside phosphorylase | NP | E |
| Pyrroline-5-carboxylase synthetase | PYCS | E |
| Pyruvate carboxylase | PC | E |
| Pyruvate decarboxylase | PDHA | E |
| Pyruvate kinase | PKLR | E |
| Quinoid dihydropteridine reductase | QDPR | E |
| Renin | REN | E |
| Replication factor A | | E |
| Replication factor C | RFC2 | E |
| Rhodopsin kinase | RHOK | E |
| Ribonucleotide reductase, RRM | | E |
| Ribosephosphate pyrophosphokinase | | E |
| Ribosomal protein L13A | RPL13A | G |
| Ribosomal protein L17 | RPL17 | G |
| Ribosomal protein S19 | RPS 19 | E |
| Ribosomal protein S4, X-linked | RPS4X | E |
| Ribosomal protein S6 kinase | RPS6KA3 | E |
| Ribosomal protein S9 | RPS9 | G |
| S-adenosylmethionine decarboxylase, | | E |
| AMD | | |
| Serine hydroxymethyltransferase | SHMT | E |
| Serotonin N-acetyltransferase | SNAT | E |
| Sorbitol dehydrogenase | SORD | E |
| Sphingomyelinase | SMPD1 | E |
| Steroid 5 alpha reductase 1 | SRD5A1 | E |
| Steroid 5 alpha reductase 2 | SRD5A2 | E |
| Steroid sulphatase | STS | E |
| Succinate dehydrogenase 1 | SDH1 | E |
| Succinate dehydrogenase 2 | SDH2 | E |
| Succinate thiokinase | | E |
| Succinic semi-aldehyde dehydrogenase | ssadh | E |
| Succinyl CoA synthase | | E |
| Sucrase | | E |
| Sulfite oxidase | SUMOX | E |
| Superoxide dismutase 1 | SOD1 | E |
| Superoxide dismutase 3 | SOD3 | E |
| TEK, tyrosine kinase, endothelial | TEK | E |
| Telomerase protein component | | E |
| Terninal deoxynucleotidyltransferase, | | E |
| TDT | | |
| Thiolase, perioxisomal | | E |
| Thiopurine S-methyltransferase | TPMT | E |
| Thymidylate synthase | TYMS | E |
| Tissue inhibitor of metalloproteinase 1, | TIMP1 | E |
| TIMP1 | | |
| Tissue inhibitor of metalloproteinase 2, | TIMP2 | E |
| TIMP2 | | |
| Tissue inhibitor of metalloproteinase 3, | TIMP3 | E |
| TIMP3 | | |
| Tissue inhibitor of metalloproteinase 4, | TIMP4 | E |
| TIMP4 | | |
| Tissue non-specific alkaline phosphatase | | E |
| Topoisomerase I | | E |
| Topoisomerase II | | E |
| Transacylase | | E |
| Transketolase | TKT | E |
| Transketolase-like 1 | TKTL1 | E |
| Triosephosphate isomerase | TPI1 | E |
| Trypsin inhibitor | | E |
| Trypsinogen 1 | TRY1 | E |
| Trypsinogen 2 | TRY2 | E |
| Tryptophan hydroxylase | TPH | E |
| Tyrosinase | TYR | E |
| Tyrosinase-related protein 1 | TYRP1 | E |
| Tyrosine amninotransferase | TAT | E |
| Tyrosine hydroxylase | TH | E |
| Ubiquitin activating enzyme, E1 | | E |
| Ubiquitin protein ligase E3A | UBE3A | E |
| UDP-glucose pyrophosphorylase | | E |
| UDP-glucuronosyltransferase 1 | ugt1d, UGT1 | E |
| UDP-glucuronosyltransferase 2 | UGT2 | E |
| Urate oxidase | UOX | E |
| Ureidopropionase | | E |
| Uridinediphosphate(UDP)-galactose-4- | GALE | E |
| Uroporphyrinogen decarboxylase | UROD | E |
| Uroporphyrinogen III synthase | UROS | E |
| Xanthine dehydrogenase | XDH | E |
| Xerodenna pigmentosum, | XPA | E |
| complementation A | | |
| Xeroderma pigmentosum, | XPB | E |
| complementation B | | |
| Xeroderma pigmentosum, | XPC | E |
| complementation C | | |
| Xeroderma pigmentosum, | | E |
| complementation D | | |
| Xeroderma pigmentosum, | | E |
| complementation E | | |
| Xeroderma pigmentosum, | XPF | E |
| complementation F | | |
| Xeroderma pigmentosum, | ERCC5 | E |
| complementation G | | |
| Xylitol dehydrogenase | | E |
| Acidic amino acid transporter | | T |
| Adaptin, beta 3A | ADTB3A | T |
| Adenine phosphoribosyltransferase | APRT | T |
| Alanine aminotransferase | | T |
| Albumin, ALB | ALB | T |
| Aldose reductase | | T |
| Alkaline phosphatase, liver/bone/kidney | ALPL | T |
| Alpha 1 acid glycoprotein | AAG; AGP | T |
| Androgen binding protein | ABP | T |
| Angiotensin receptor 1 | AGTR1 | T |
| Angiotensin receptor 2 | AGTR2 | T |
| Antidiuretic homione receptor | ADHR | T |
| Apolipoprotein (a) | LPA | T |
| Apolipoprotein A 4 | APOA4 | T |
| Apolipoprotein A I | APOA1 | T |
| Apolipoprotein A II | APOA2 | T |
| Apolipoprotein B | APOB | T |
| Apolipoprotein Cl | APOC1 | T |
| Apolipoprotein C2 | APOC2 | T |
| Apolipoprotein C3 | APOC3 | T |
| Apolipoprotein D | APOD | T |
| Apolipoprotein E | APOE | T |
| Apolipoprotein H | APOH | T |
| Aquaporin 1 | AQP1 | T |
| Aquaporin 2 | AQP2 | T |
| Aryl hydrocarbon receptor | AHR | T |
| Aryl hydrocarbon receptor nuclear | ARNT | T |
| translocator | | |
| Aspartate transaminase | | T |
| Bestrophin | VMD2 | T |
| Bile salt export pump | BSEP, PFIC2 | T |
| Biliverdin reductase | | T |
| Ca(2+) transporting ATPase, fast twitch | ATP2A1 | T |
| Ca(2+) transporting ATPase, slow twitch | ATP2A2 | T |
| Calcium sensing receptor | CASR | T |
| Calmodulin dependant kinase | | T |
| Canalicular multispecific organic anion | CMOAT | T |
| Carnitine transporter protein | CDSP, SCD | T |
| Chediak-Higashi syndrome 1 gene | CHS1 | T |
| Cholesterol ester transfer protein | CETP | T |
| Clathrin | | T |
| Cortico-steroid binding protein | | T |
| Corticotrophin-releasing hormone | CRH | T |
| Corticotrophin-releasing hormone receptor | CRHR1 | T |
| Cubilin | CUBN | T |
| Cystatin B | CSTB | T |
| Cystatin C | CST3 | T |
| Cysteine-rich intestinal protein | | T |
| Cystinosin | CTNS | T |
| Diastrophic dysplasia sulfate transporter | DTD | T |
| Duffy blood group | FY | T |
| Electron-transfering-flavoprotein alpha | ETFA | T |
| Electron-transfering-flavoprotein beta | ETFB | T |
| Emerin | EMD | T |
| Enteric lipase | | T |
| Faciogenital dysplasia | FGD1, FGDY | T |
| Fanconi anemia, complementation | FANCA | T |
| group A | | |
| Fanconi anemia, complementation | FANCC | T |
| group C | | |
| Fanconi anemia, complemnentation | FANCD | T |
| group D | | |
| Fatty acid binding proteins FABP1 | | T |
| Fatty acid binding proteins FABP2 | FABP2 | T |
| Fatty acid binding proteins FABP3 | | T |
| Fatty acid binding proteins FABP4 | | T |
| Fatty acid binding proteins FABP5 | | T |
| Fatty acid binding proteins FABP6 | | T |
| Ferritin, H subunit | | T |
| Ferritin, L subunit | FTL | T |
| Fucosyltransferase 2 | FUT2 | T |
| Fucosyltransferase 3 | FUT3 | T |
| Fucosyltransferase 6 | FUT6 | T |
| Furin | | T |
| Gamma-glutamnyl carboxylase | GGCX | T |
| Gamma-glutamyltransferase 1 | GGT1 | T |
| Gamma-glutamyltransferase 2 | GGT2 | T |
| Gap junction protein alpha 1 | GJA1 | T |
| Gap junction protein alpha 3 | GJA3 | T |
| Gap junction protein alpha 8 | GJA8 | T |
| Gap junction protein beta 1 | GJB1 | T |
| Gap junction protein beta 2 | GJB2 | T |
| Gap junction protein beta 3 | GJB3 | T |
| Gastric inhibitory polypeptide GIP | GIP | T |
| Gastric inhibitory polypeptide receptor, | GIPR | T |
| GIPR | | |
| Gastric lipase, LIPF | | T |
| Gastrin releasing peptide | GRP | T |
| Gastrin releasing peptide receptor | GRPR | T |
| Glucagon synthase | | T |
| Glutamine transporter | | T |
| Glutathione | GSH | T |
| Guanylin | GUCA2 | T |
| Haem oxygenase | | T |
| Haemoglobin alpha 1 | HBA1 | T |
| Haemoglobin alpha 2 | HBA2 | T |
| Haemoglobin beta | HBB | T |
| Haemoglobin delta | HBD | T |
| Haemoglobin epsilon | | T |
| Haemoglobin gamma A | HBG1 | T |
| Haemoglobin gamma B | HBG2 | T |
| Haemoglobin gamma G | HBGG | T |
| Hcmochromatosis | HFE | T |
| Hermansky-pudlak syndrome gene | HPS | T |
| Histidine-rich glycoprotein | HRG | T |
| Huntingtin | HD | T |
| Hyaluronidase | | T |
| Intestinal alkaline phosphatase IAP | | T |
| Kell blood group precursor | XK, KEL | T |
| Lactotransferrin | LTF | T |
| Lipoprotein receptor, Low Density | LDLR | T |
| Lipoprotein, High Density | HDLDT1 | T |
| Lipoprotein, Intermediate Density | | T |
| Lipoprotein, Low Density 1 | | T |
| Lipoprotein, Low Density 2 | | T |
| Lipoprotein, Very Low Density | VLDLR | T |
| Long QT-type 2 potassium channels | LQT2, KCNH2 | T |
| Low density lipoprotein receptor-related | LRP | T |
| protein precursor | | |
| Mannosyl (alpha-1,6-)-glycoprotein | MGAT2 | T |
| beta-1,2-acetylglucosaminyltransferase | | |
| Marenostrin | MEFV | T |
| Melanocortin 1 receptor | MC1R | T |
| Melanocortin 2 receptor | MC2R | T |
| Melanocortin 4 receptor | MC4R | T |
| Metallothionein | | T |
| Microsomal triglyceride transfer protein | MTP | T |
| Mucin 18 | MUC18 | T |
| Mucin, MUC2 | | T |
| Mucin, MUC5AC | | T |
| Mucin, MUC6 | | T |
| Mulibrey nanism | MUL | T |
| Myocilin | MYOC | T |
| Myoglobin | | T |
| Myopia 1 | MYP1 | T |
| Myopia 2 | MYP2 | T |
| Na+/H+ exchanger 1 | NHE1 | T |
| Na+/H+ exchanger 2 | NHE2 | T |
| Na+/H+ exchanger 3 | NHE3 | T |
| Na+/H+ exchanger 4 | NHE4 | T |
| Na+/H+ exchanger 5 | NHE5 | T |
| Na + coupled glucose/galactose | | T |
| transporter | | |
| Nephrolithiasis 2 | NPHL2 | T |
| Nephronophthisis 1 | NPHP1 | T |
| Ncphronophthisis 2 | NPHP2 | T |
| Nephrosis 1 | NPHS1 | T |
| Neuraminidase sialidase | NEU | T |
| Niemann-Pick disease protein | NPC1 | T |
| Nucleophosmin | NPM1 | T |
| Palmitoyl-protein thioesterase | PPT | T |
| Pancreatic colipase | | T |
| Pendrin, PDS | PDS | T |
| Pepsin | | T |
| Peptidascs A | | T |
| Peptidascs B | | T |
| Peptidases C | | T |
| Peptidases D | PEPD | T |
| Peptidases E | | T |
| Peptidases S | | T |
| Peroxisomal membrane protein 3 | PXMP3 | T |
| Peroxisome biogenesis factor 1 | PEX1 | T |
| Peroxisome biogenesis factor 6 | PEX6 | T |
| Peroxisome biogenesis factor 7 | PEX7 | T |
| Peroxisome biogencsis factor 19 | PEX19 | T |
| Peroxisome proliferative activated | PPARA | T |
| receptor, | | |
| Peroxisome proliferative activated | PPARG | T |
| receptor, | | |
| Peroxisome receptor 1 | PXR1 | T |
| P-glycoprotein 1 | PGY1 | T |
| P-glycoprotein 3 | PGY3 | T |
| Phosphomannomutase-2 | PMM2 | T |
| Phosphomannose isomerase-1, PMI1 | MPI | T |
| Plakophilin 1 | PKP1 | T |
| Platelet glutaminase | GLS | T |
| Platelet monoamine oxidase | | T |
| Plectin 1 | PLEC1 | T |
| Polycystic kidney and hepatic disease 1 | PKHD1 | T |
| Polycystin 1 | PKD1 | T |
| Polycystin 2 | PKD2 | T |
| Polymorphonuclear elastase | | T |
| Preproglucagon | | T |
| Preproinsulin | | T |
| Presenilin 1 | PSEN1 | T |
| Presenilin 2 | PSEN2 | T |
| Prostaglandin I2 receptor | | T |
| Protease inhibitor 1 | | T |
| Renal glutaminase | | T |
| Retinaldehyde binding protein 1 | RLBP1 | T |
| Retinol binding protein 1 | | T |
| Retinol binding protein 2 | | T |
| Retinol binding protein 4 | RBP4 | T |
| Rhesus blood group, CcEe antigens | RHCE | T |
| Rhesus blood group, D antigen | RHD | T |
| Rhesus blood group-associated | RHAG | T |
| glycoprotein | | |
| Salivary amylase, AMY1 | | T |
| Secretin | SCT | T |
| Secretin receptor, SCTR | SCTR | T |
| Serum amyloid A | SAA | T |
| Serum amyloid P | SAP | T |
| Sex honnone binding globulin, SHBG | | T |
| Solute carrier family 1 (amino acid | SLC1A6 | T |
| transporter), member 6 | | |
| Solute carrier family 1 (glial high affinity | SLC1A3 | T |
| transporter), member 3 | | |
| Solute carrier family 1 (glutamate | SLC1A1 | T |
| transporter), member 1 | | |
| Solute carrier family 1 (glutamate | SLC1A2 | T |
| transporter), member 2 | | |
| Solute carrier family 1 (neutral amino acid | SLC1A4 | T |
| transporter), member 4 | | |
| Solute carrier family 10 (sodium/bile acid | SLC10A1 | T |
| cotransporter family), member 1 | | |
| Solute carrier family 10 (sodium/bile acid | SLC10A2 | T |
| cotransporter family), membcr 2 | | |
| Solute carrier family 12, member 1 | SLC12A1 | T |
| Solute carrier family 12, member 2 | SLC12A2 | T |
| Solute carrier family 12, member 3 | SLC12A3 | T |
| Solute carrier family 14, member 2 | SLC14A2 | T |
| Solute carrier family 15 (H+/peptide | SLC15A1 | T |
| intestinal), member 1 | | |
| Solute carrier family 15 (H+/peptide | SLC15A2 | T |
| kidney), member 2 | | |
| Solute carrier family 16 (monocarboxylate | SLC16A1 | T |
| transporter), member 1 | | |
| Solute carrier family 16 (monocarboxylate | SLC16A7 | T |
| transporter), member 7 | | |
| Solute carrier family 17, member 1 | SLC17A1 | T |
| Solute carrier family 17, member 2 | SLC17A2 | T |
| Solute carrier family 18, member 3 | SLC18A3 | T |
| Solute carrier family 19 (folate | SLC19A1 | T |
| transporter), member 1 | | |
| Solute carrier family 2 (facilitated glucose | SLC2A1 | T |
| transporter), member 1 | | |
| Solute carrier family 2 (facilitated glucose | SLC2A2 | T |
| transportEr), member 2 | | |
| Solute carrier family 2 (facilitated glucose | SLC2A3 | T |
| transporter), member 3 | | |
| Solute carrier family 2 (facilitated glucose | SLC2A4 | T |
| transporter), member 4 | | |
| Solute carrier family 2 (facilitated glucose | SLC2A5 | T |
| transporter), member 5 | | |
| Solute carrier family 20, member 1 | SLC20A1 | T |
| Solute carrier family 20, member 2 | SLC20A2 | T |
| Solute carrier family 20, member 3 | SLC20A3 | T |
| Solute carrier family 21, member 2 | SLC21A2 | T |
| Solute carrier family 21, member 3 | SLC21A3 | T |
| Solute carrier family 22, member 1 | SLC22A1 | T |
| Solute carrier family 22, member 2 | SLC22A2 | T |
| Solute carrier family 22, member 5 | SLC22A5 | T |
| Solute carrier family 25, member 12 | SLC25A12 | T |
| Solute carrier family 3 (facilitated glucose | SLC3A1 | T |
| transporter), member 1 | | |
| Solute carrier family 4 (anion | SLC4A1 | T |
| exchanger), 1 | | |
| Solute carrier family 4 (anion | SLC4A2 | T |
| exchanger), 2 | | |
| Solute carrier family 4 (anion | SLC4A3 | T |
| exchanger), 3 | | |
| Solute carrier family 5 (sodium/glucose | SLC5A1 | T |
| transporter), member 1 | | |
| Solute carrier family 5 (sodium/glucose | SLC5A2 | T |
| transporter), member 2 | | |
| Solute carrier family 5 (sodium/glucose | SLC5A5 | T |
| transporter), member 5 | | |
| Solute carrier family 5, member 3 | SLC5A3 | T |
| Solute carrier family 6 (GAMMA- | SLC6A1 | T |
| AMINOBUTYRIC ACID transporter), | | |
| member | | |
| Solute carrier family 6 (neurotransmitter | SLC6A3 | T |
| transporter, dopamine), member 3 | | |
| Solute carrier family 6 (neurotransmitter | SLC6A2 | T |
| transporter, noradrenaline), member 2 | | |
| Solute carrier family 6 (neurotransmitter | SLC6A4 | T |
| transporter, serotonin), member 4 | | |
| Solute carrier family 6, member 10 | SLC6A10 | T |
| Solute carrier family 6, member 6 | SLC6A6 | T |
| Solute carrier family 6, member 8 | SLC6A8 | T |
| Solute carrier family 7(amino acid | SLC7A1 | T |
| transporter), member 1 | | |
| Solute carrier family 7(amino acid | SLC7A2 | T |
| transporter), member 2 | | |
| Solute carrier family 7(amino acid | SLC7A7 | T |
| transporter), member 7 | | |
| Solute carrier family 8 (sodium/calcium | SLC8A1 | T |
| member 1 | | |
| Sorcin | SRI | T |
| Steroidogenic acute regulatory protein | STAR | T |
| Sterol carrier protein 2 | SCP2 | T |
| Stratum corneum chymotryptic enzyme | | T |
| Sucrase-isomaltase | S1 | T |
| Surfactant pulmonary-associated | SFTPA1 | T |
| protein A1 | | |
| Surfactant pulmonary-associated | SFTPA2 | T |
| protein A2 | | |
| Surfactant pulmonary-associated | SFTPB | T |
| protein B | | |
| Surfactant pulmonary-associated | SFTPC | T |
| protein C | | |
| Surfactant pulmonary-associated | SFTPD | T |
| protein D | | |
| Survival of motor neuron 1, telomeric | SMN1 | T |
| Tetranectin | TNA | T |
| Thyroxin-binding globulin | TBG | T |
| Tocopherol (alpha) transfer protein | TTPA | T |
| Transcobalamin 1, TCN1 | T | |
| Transcobalamin 2, TCN2 | TCN2 | T |
| Transthyretin | TTR | T |
| Trehalase | | T |
| Trypsinogen activation peptide | | T |
| Uncoupling protein 1 | | T |
| Uncoupling protein 3 | UCP3 | T |
| Uteroglobin | UGB | T |
| Vitelliforn macular dystrophy, | VMD1 | T |
| atypical gene | | |
| Vitronectin receptor, alpha | VNRA | T |
| Von Willebrand factor | VWF | T |
| Achromatopsia 2 | ACHM2 | S |
| Actin, alpha, skeletal | ACTA1 | S |
| Actin, alpha, smooth, aortic | ACTA2 | S |
| Actin, alpha, cardiac | ACTC | S |
| Actin, beta | ACTB | S |
| Actin, gamma 2 | ACTG2 | S |
| Adducin, alpha | ADD1 | S |
| Adducin, beta | ADD2 | S |
| Amelogenin | AMELX | S |
| Ankyrin 1 | ANK1 | S |
| Ankyrin 2 | ANK2 | S |
| Ankyrin 3 | ANK3 | S |
| Apaf-1 | | S |
| Arrestin | SAG | S |
| Blue cone pigment | BCP | S |
| Chloride channel 1, skeletal muscle | CLCN1 | S |
| Chloride channel 5 | CLCN5 | S |
| Chloride channel KB | CLCNKB | S |
| Choroideremia gene | CHM | S |
| Cofilin | | S |
| Collagen I alpha 1 | COL1A1 | S |
| Collagen I alpha 2 | COL1A2 | S |
| Collagen II alpha 1 | COL2A1 | S |
| Collagen III alpha 1 | COL3A1 | S |
| Collagen IV alpha 1 | COL4A1 | S |
| Collagen IV alpha 2 | COL4A2 | S |
| Collagen IV alpha 3 | COL4A3 | S |
| Collagen IV alpha 4 | COL4A4 | S |
| Collagen IV alpha 5 | COL4A5 | S |
| Collagen IV alpha 6 | COL4A6 | S |
| Collagen IX alpha 2 | COL9A2, | S |
| | EDM2 | |
| Collagen IX alpha 3 | COL9A3 | S |
| Collagen receptor | COLR | S |
| Collagen V alpha 1 | COL5A1 | S |
| Collagen V alpha 2 | COL5A2 | S |
| Collagen VI alpha 1 | COL6A1 | S |
| Collagen VI alpha 2 | COL6A2 | S |
| Collagen VI alpha 3 | COL6A3 | S |
| Collagen VII alpha 1 | COL7A1 | S |
| Collagen X alpha 1 | COL10A1 | S |
| Collagen X alpha 1 | COL11A1 | S |
| Collagen XI alpha 2 | COL11A2 | S |
| Collagen XVII alpha 1 | COL17A1 | S |
| Cryptochrome 1 | CRY1 | S |
| Cryptochrome 2 | CRY2 | S |
| Crystallin, alpha A | CRYAA | S |
| Crystallin, alpha B | CRYAB | S |
| Crystallin, beta B2 | CRYBB2 | S |
| Crystallin, gamma A | CRYGA | S |
| Desmin | DES | S |
| DNA damage binding protein, DDB1 | DDB1 | S |
| DNA damage binding protein, DDB2 | DDB2 | S |
| DNA-damage-inducible transcript 3 | DDIT3 | S |
| Doublecortin, DCX | DCX | S |
| Dyskerin | DKC1 | S |
| Dystonia 1 | DYT1 | S |
| Dystonia 3 | DYT3 | S |
| Dystonia 6 | DYT6 | S |
| Dystonia 7 | DYT7 | S |
| Dystonia 9 | CSE | S |
| Dystrophin | DMD | S |
| Dystrophin-associated glycoprotein 35kD, | SGCD | S |
| Dystrophin-associated glycoprotein 35kD, | SGCG | S |
| Dystrophin-associated glycoprotein 43kD | SGCB | S |
| Dystrophin-associated glycoprotein 50kD | SGCA | S |
| Ectodermal Dysplasia 1 gene | ED1 | S |
| Elastin | ELN | S |
| Endocardial fibroelastosis 2 gene | EFE2 | S |
| Endoglin ENG | S | |
| Erythrocyte membrane protein band 4.1 | EPB41 | S |
| Erythrocyte membrane protein band 4.2 | EPB42 | S |
| Erythrocyte membrane protein band 7.2 | EPB72 | S |
| Exostosin 1 | EXT1 | S |
| Exostosin 2 | EXT2 | S |
| Exostosin 3 | EXT3 | S |
| Eye colour gene 3 (brown) | EYCL3 | S |
| Fibrinogen alpha | FGA | S |
| Fibrinogen beta | FGB | S |
| Fibrinogen gamma | FGG | S |
| Glycophorin A | GYPA | S |
| Glycophorin B | GYPB | S |
| Glycophorin C | GYPC | S |
| Green cone pigment | GCP | S |
| Keratin 1 | KRT1 | S |
| Keratin 10 | KRT10 | S |
| Keratin 11 | KRT11 | S |
| Keratin 12 | KRT12 | S |
| Keratin 13 | KRT13 | S |
| Keratin 14 | KRT14 | S |
| Keratin 15 | KRT15 | S |
| Keratin 16 | KRT16 | S |
| Keratin 17 | KRT17, | S |
| | PCHC1 | |
| Keratin 18 | KRT18 | S |
| Keratin 2 | KRT2 | S |
| Keratin 3 | KRT3 | S |
| Keratin 4 | KRT4 | S |
| Keratin 5 | KRT5 | S |
| Keratin 6 | KRT6 | S |
| Keratin 7 | KRT7 | S |
| Keratin 8 | KRT8 | S |
| Keratin 9 | KRT9 | S |
| Keratin, hair acidic 1 | KRTHA1 | S |
| Keratin, hair basic 2 | KRTHB1 | S |
| Keratin, hair basic 6 | KRTHB6 | S |
| Loricrin | LOR | S |
| Microtuble associated protein | MAP | S |
| Moesin, MSN | | S |
| Myomesin 1 | MYOM1 | S |
| Myomesin 2 | MYOM2 | S |
| Myelin basic protein | | S |
| Myelin protein peripheral 22 | PMP22 | S |
| Myelin protein zero | MPZ | S |
| Myosin 15 | MYO15 | S |
| Myosin 5A | MYO5A | S |
| Myosin 6 | MYO6 | S |
| Myosin 7A | MYO7A | S |
| Myosin, cardiac | MYH7 | S |
| Myosin, light chain 2 | MYL2 | S |
| Myosin, light chain 3 | MYL3 | S |
| Myosin-binding protein C, cardiac | MYBPC3 | S |
| Myotubularin | MTM1 | S |
| Nebulin | NEB | S |
| Neurofilament protein, heavy | NFH | S |
| Neurofilament protein, NF125 | NF150 | S |
| Neurofilament protein, NF200 | NF200 | S |
| Neurofilament protein, NF68 | NF68 | S |
| Ocular albinism I | OA1 | S |
| Oculocutaneous albinism II | OCA2 | S |
| Ostcocalcin | | S |
| Peripherin, PRPH | | S |
| Peroxisomal membrane protein 1 | PXMP1 | S |
| Persyn | | S |
| Proline-rich protein BstNI subfamily 1 | PRB1 | S |
| Proline-rich protein BstNI subfamily 3 | PRB3 | S |
| Proline-rich protein BstNI subfamily 4 | PRB4 | S |
| Radixin/ RDX | S | |
| Red cone pigment | RCP | S |
| Retinal pigment epithelium specific | RPE65 | S |
| protein | | |
| Retinitis pigmentosa gene 1 | RP1 | S |
| Retinitis pigmentosa gene 2 | RP2 | S |
| Retinitis pigmentosa gene 3 | RP3 | S |
| Retinitis pigmentosa gene 6 | RP6 | S |
| Retinitis pigmentosa gene 7 | RP7, RDS | S |
| Rhodopsin | RPHO | S |
| Rod outer segment membrane protein 1 | ROM1 | S |
| Semaphorin A4 | SEMA4 | S |
| Semaphorin A5 | SEMA5 | S |
| Semaphorin D | | S |
| Semaphorin E | SEMAE | S |
| Semaphorin F | SEMA3/F | S |
| Semaphorin W | SEMAW | S |
| Small nuclear ribonucleoprotein | SNRPN | S |
| polypeptide N | | |
| Spectrin alpha | SPTA1 | S |
| Spectrin beta | SPTB | S |
| Talin, TLN | | S |
| Tau protein | MAPT | S |
| Tenascin (cytotactin) | | S |
| Tenascin XA | TNXA | S |
| Titin | TTN | S |
| Tropomyosin 1 alpha | TPM1 | S |
| Tropomyosin 3 (non-muscle) | TPM3 | S |
| Troponin C | | S |
| Troponin I | TNNI3 | S |
| Troponin T2, cardiac | TNNT2 | S |
| Tubulin | | S |
| Undulin 1 | COL14A1 | S |
| Usher syndrome 2A | USH2A | S |
| Villin | | S |
| Vinculin | | S |
| Wolfram syndrome 1 gene | WFS1 | S |
| Zinc finger protein 198 | ZIC198 | S |
| Zinc finger protein 2 | ZIC2 | S |
| Zinc finger protein 3 | ZIC3 | S |
| Zinc finger protein HRX | ALL1 | I |
| Alpha 2 macroglobulin | A2M | I |
| Annexin 1 | ANX 1 | I |
| Apoptosis antigen 1 | APT1 | I |
| Apoptosis antigen ligand 1 | APT1LG1 | I |
| Apoptosis-inducing factor | AIF | I |
| ATP-binding cassette transporter 7 | ABC7 | I |
| Attractin | | I |
| Autoimmune regulator, AIRE AIRE | I | |
| B-cell CLL/lymphoma 1 | BCL1 | I |
| B-cell CLL/lymphoma 10 | BCL10 | I |
| B-cell CLL/lymnphoma 3 | BCL3 | I |
| B-cell CLL/lymphoma 4 | BCL4 | I |
| B-cell CLL/lymphoma 5 | BCL5 | I |
| B-cell CLL/lymphoma 6 | BCL6 | I |
| B-cell CLL/lymphoma 7 | BCL7 | I |
| B-cell CLL/lymphoma 8 | BCL8 | I |
| B-cell CLL/lymphoma 9 | BCL9 | I |
| beta 2 microglobulin | B2M | I |
| Bradykinin receptor B1 | | I |
| Bradykinin receptor B2 | | I |
| Calcineurin A1 | CALNA1 | I |
| Calcineurin A2 | CALNA2 | I |
| Calcineurin A3 | CALNA3 | I |
| Calcineurin B | | I |
| Catalase | CAT | I |
| CD1 | CD1 | I |
| CD10 | CD10 | I |
| CD100 | CD100 | I |
| CD101 | CD101 | I |
| CD103 | CD103 | I |
| CD106 | CD106 | I |
| CD107 | CD107 | I |
| CD108 | CD108 | I |
| CD109 | CD109 | I |
| CD110 | CD110 | I |
| CD111 | CD111 | I |
| CD112 | CD112 | I |
| CD113 | CD113 | I |
| CD114 | CD114 | I |
| CD115 | CD115 | I |
| CD116 | CD116 | I |
| CD117 | CD117 | I |
| CD118 | CD118 | I |
| CD119 | CD119 | I |
| CD12 | CD12 | I |
| CD120 | CD120 | I |
| CD121 | CD121 | I |
| CD122 | CD122 | I |
| CD123 | CD123 | I |
| CD124 | CD124 | I |
| CD125 | CD125 | I |
| CD126 | CD126 | I |
| CD127 | CD127 | I |
| CD128 | CD128 | I |
| CD129 | CD129 | I |
| CD13 | CD13 | I |
| CD130 | CD130 | I |
| CD131 | CD131 | I |
| CD132 | CD132 | I |
| CD133 | CD133 | I |
| CD134 | CD134 | I |
| CD135 | CD135 | I |
| CD136 | CD136 | I |
| CD137 | CD137 | I |
| CD138 | CDl38 | I |
| CD139 | CD139 | I |
| CD14 | CD14 | I |
| CD140 | CD140 | I |
| CD141 | CD141 | I |
| CD142 | CD142 | I |
| CD143 | CD143 | I |
| CD144 | CD144 | I |
| CD145 | CD145 | I |
| CD147 | CD147 | I |
| CD148 | CD148 | I |
| CD149 | CD149 | I |
| CD15 | CD15 | I |
| CD150 | CD150 | I |
| CD151 | CD151 | I |
| CD152 | CD152 | I |
| CD153 | CD153 | I |
| CD154 | CD154 | I |
| CD155 | CD155 | I |
| CD156 | CD156 | I |
| CD157 | CD157 | I |
| CD158 | CD158 | I |
| CD159 | CD159 | I |
| CD160 | CD160 | I |
| CD161 | CD161 | I |
| CD162 | CD162 | I |
| CD163 | CD163 | I |
| CD164 | CD164 | I |
| CD165 | CD165 | I |
| CD166 | CD166 | I |
| CD17 | CD17 | I |
| CD19 | CD19 | I |
| CD2 | CD2 | I |
| CD20 | CD20 | I |
| CD22 | CD22 | I |
| CD23 | CD23 | I |
| CD24 | CD24 | I |
| CD25 | CD25 | I |
| CD26 | CD26 | I |
| CD27 | CD27 | I |
| CD28 | CD28 | I |
| CD3 | CD3 | I |
| CD30 | CD30 | I |
| CD31 | CD31 | I |
| CD33 | CD33 | I |
| CD34 | CD34 | I |
| CD36 | CD36 | I |
| CD37 | CD37 | I |
| CD38 | CD38 | I |
| CD39 | CD39 | I |
| CD4 | CD4 | I |
| CD40 | CD40 | I |
| CD41 | CD41 | I |
| CD42 | CD42 | I |
| CD43 | CD43 | I |
| CD44 | CD44 | I |
| CD45 | CD45 | I |
| CD46 | CD46 | I |
| CD47 | CD47 | I |
| CD48 | CD48 | I |
| CD5 | CD5 | I |
| CD50 | CD50 | I |
| CD52 | CD52 | I |
| CD53 | CD53 | I |
| CD55 | CD55 | I |
| CD57 | CD57 | I |
| CD58 | CD58 | I |
| CD59 | CD59 | I |
| CD6 | CD6 | I |
| CD60 | CD60 | I |
| CD63 | CD63 | I |
| CD65 | CD65 | I |
| CD66 | CD66 | I |
| CD67 | CD67 | I |
| CD68 | CD68 | I |
| CD69 | CD69 | I |
| CD7 | CD7 | I |
| CD70 | CD70 | I |
| CD71 | CD71 | I |
| CD72 | CD72 | I |
| CD73 | CD73 | I |
| CD74 | CD74 | I |
| CD75 | CD75 | I |
| CD76 | CD76 | I |
| CD77 | CD77 | I |
| CD78 | CD78 | I |
| CD79 | CD79 | I |
| CD8 | CD8 | I |
| CD80 | CD80 | I |
| CD81 | CD81 | I |
| CD83 | CD83 | I |
| CD84 | CD84 | I |
| CD85 | CD85 | I |
| CD86 | CD86 | I |
| CD88 | CD88 | I |
| CD89 | CD89 | I |
| CD9 | CD9 | I |
| CD90 | CD90 | I |
| CD91 | CD91 | I |
| CD92 | CD92 | I |
| CD93 | CD93 | I |
| CD94 | CD94 | I |
| CD96 | CD96 | I |
| CD97 | CD97 | I |
| CD98 | CD98 | I |
| CD99 | CD99 | I |
| Chemokine MCAF | MCAF | I |
| Chemokine receptor CCR2 | CCR2 | I |
| Chemokine receptor CCR3 | CCR3 | I |
| Chemokine receptor CCR5 | CCR5 | I |
| Chemokine receptor CXCR1 | CXCR1 | I |
| Chemokine receptor CXCR2 | CXCR2 | I |
| Chemokine receptor CXCR4 | CXCR4 | I |
| Cholesterylester hydrolase | | I |
| Chondritin Sulphate A—placental receptor | | I |
| Cochlin | COCH | I |
| Complement component C1 inhibitor | C1NH | I |
| Complement component C1qa | C1QA | I |
| Complement component C1qb | C1QB | I |
| Complement component C1qg | C1QG | I |
| Complement component C1r | C1R | I |
| Complement component C1s | C1S | I |
| Complement component C2 | C2 | I |
| Complement component C3 | C3 | I |
| Complement component C4A | C4A | I |
| Complement component C4B | C4B | I |
| Complement component C5 | C5 | I |
| Complement component C6 | C6 | I |
| Complement component C7 | C7 | I |
| Complement component C8 | C8 | I |
| Complement component C9 | C9 | I |
| Complement component receptor 1 | CR1 | I |
| Complement component receptor 2 | CR2 | I |
| Complement component receptor 3 | CR3 | I |
| Corticosteroid nuclear receptor | | I |
| Cortisol receptor | | I |
| C-reactive protein CRP | | I |
| Cyclophilin | | I |
| Cytokine-suppressive antiinflammatory | CSBP1 | I |
| drug-binding protein 1 | | |
| Cytokine-suppressive antiinflammatory | CSBP2 | I |
| drug-binding protein 2 | | |
| DAX1 nuclear receptor | DAX1 | I |
| Endo-P-D-glucuronicase | | I |
| Erythropoietin | EPO | I |
| Erythropoietin receptor | EPOR | I |
| Factor 1 (No. one) | F1 | I |
| Factor B, properdin | | I |
| Factor D | | I |
| Factor H | HF1 | I |
| Factor I (letter I) | 1F | I |
| Factor III | F3 | I |
| Factor IX | F9 | I |
| Factor V | F5 | I |
| Factor VII | F7 | I |
| Factor VIII | F8 | I |
| Factor X | F10 | I |
| Factor XI | F11 | I |
| Factor XII | F12 | I |
| Factor XIII A & B | F13A & F13B | I |
| Fc receptor | | I |
| Follicular lymphoma variant | FVT1 | I |
| translocation 1 | | |
| Gastrointestinal tumor-associated | GA733 | I |
| antigen 1 | | |
| Growth-regulated protein precursor, GRO | GRO | I |
| Haptoglobin, alpha 1 | HPA1 | I |
| Haptoglobin, alpha 2 | HPA2 | I |
| Haptoglobin, beta | HPB | I |
| Heat shock protein, HSP60 | | I |
| Heat shock protein, HSP70 | | I |
| Heat shock protein, HSP90 | | I |
| Heat shock protein, HSPA1 | | I |
| Heat shock protein, HSPA2 | | I |
| Hemopexin | HPX | I |
| Heparin Cofactor II | HCF2 | I |
| Hepatitis B virus integration site 1 | HVBS1 | I |
| Hepatitis B virus integration site 2 | HVBS6 | I |
| Histatin 1 | | I |
| Histatin 2 | | I |
| Histatin 3 | HTN3 | I |
| HLA-B associated transcript 1 | BAT1 | I |
| IC7 A and B | | I |
| Immunoglobulin alpha (IgA) | IGHA | I |
| Immunoglobulin gamma (IgG) 2 | IGHG2 | I |
| Immunoglobulin delta (IgD) | IGHD | I |
| Immunoglobulin epsilon (IgE) | IGHE | I |
| Immunoglobulin E (IgE) reponsiveness | IGER | I |
| gene | | |
| Immunoglobulin E (IgE) serum | IGES | I |
| concentration regulator gene | | |
| Immunoglobulin heavy mu chain | IGHM | I |
| Immunoglobulin J polypeptide | IGJ | I |
| Immunoglobulin kappa constant region | IGKC | I |
| Immunoglobulin kappa variable region | IGKV | I |
| Intercellular adhesion molecule 1 | ICAM1 | I |
| Intercellular adhesion molecule 2 | ICAM2 | I |
| Intercellular adhesion molecule 3 | ICAM3 | I |
| Interferon alpha | IFNA1 | I |
| Interferon beta | IFNB | I |
| Interferon gamma | IFNG | I |
| Interferon gamma receptor 1 | IFNGR1 | I |
| Interferon gamma receptor 2 | IFNGR2 | I |
| Interferon regulatory factor 1 | IRF 1 | I |
| Interferon regulatory factor 4 | IRF4 | I |
| Interleukin(IL) 1, receptor | IL1R | I |
| Interleukin(IL) 1, alpha | IL1A | I |
| Interleukin(IL) 1, beta | IL1B | I |
| Interleukin(IL) 10 | IL10 | I |
| Interleukin(IL) 10 receptor | IL10R | I |
| Interleukin(IL) 11 | IL11 | I |
| Interleukin(IL) 11 receptor | IL11R | I |
| Interleukin(IL) 12 | IL12 | I |
| Interleukin(IL) 12 receptor, beta 1 | IL12RB1 | I |
| Interleukin(IL) 13 | IL13 | I |
| Interleukin(IL) 13 receptor | IL13R | I |
| Interleukin(lL) 2 | IL2 | I |
| Interleukin(IL) 2 receptor, alpha | IL2RA | I |
| Interleukin(IL) 2 receptor, gamma | IL2RG | I |
| Interleukin(IL) 3 | IL3 | I |
| Interleukin(IL) 3 receptor | IL3R | I |
| Interleukin(IL) 4 | IL4 | I |
| Interleukin(IL) 4 receptor | IL4R | I |
| Interleukin(IL) 5 | IL5 | I |
| Interleukin(IL) 5 receptor | IL5R | I |
| Interleukin(IL) 6 | IL6 | I |
| Interleukin(IL) 6 receptor | IL6R | I |
| Interleukin(IL) 7 | IL7 | I |
| Interleukin(IL) 7 receptor | IL7R | I |
| Interleukin(IL) 8 | IL8 | I |
| Interleukin(IL) 8 receptor | IL8R | |
| Interleukin(IL) 9 | IL9 | I |
| Interleukin(IL) 9 receptor | IL9R | I |
| Interleukin(IL) receptor antagonist 1 | IL1RN, IL1RA | I |
| Kallikrein 3 | KAK3 | I |
| Kininogen, High molecular weight | KNG | I |
| Lectin, mannose-binding 1 | LMAN1 | I |
| Lectin, mannose-binding 2 | MBL2 | I |
| Leukin | | I |
| Leukocyte-specific transcript 1 | LST-1 | I |
| Leukotriene A4 hydrolase | | I |
| Leukotriene B4 receptor | | I |
| Leukotriene C4 receptor | | I |
| Leukotriene D4/E4 receptor | | I |
| LIM-Kinase 1 (LINK-1) | | I |
| Lipocortin 1 | ANX4 | I |
| Lipoprotein lipase | LPL | I |
| Lipoprotein-associated coagulation factor | LAC1 | I |
| Lipoxygenase 12 (platelets) | LOG12 | I |
| Lipoxygenase 5 (leukocytes) | | I |
| Lymphoblastic leukemia derived | LYL1 | I |
| sequence 1 | | |
| Lymphocyte-specific protein tyrosine | LCK | I |
| kinase lymphotoxin | | |
| Lysozyme | LYZ | I |
| Macrophage activating factor | MAF | I |
| Macrophage inflammatory protein-1 | MIP1 | I |
| Macrophage inflammatory protein-1 | | I |
| receptor | | |
| Macrophage inflammatory protein-2 | MIP2 | I |
| Macrophage inflammatory protein-2 | | I |
| receptor | | |
| Malignant proliferation, eosinophil gene | MPE | I |
| Mannose binding protein | MBP | I |
| MHC Class I: A | | I |
| MHC Class I: B | | I |
| MHC Class I: C | | I |
| MHC Class I: LMP-2, LMP-7 | | I |
| MHC Class I: Tap 1 | ABCR, TAP1 | I |
| MHC Class II: DP | HLA-DPB1 | I |
| MHC Class II: DQ | | I |
| MHC Class II: DR | | I |
| MHC Class II: Tap2 | TAP2, PSF2 | I |
| MHC Class II: Complementation group A | MHC2TA | I |
| MHC Class II: Complementation group B | rfxank | I |
| MHC Class II: Complementation group C | RFX5 | I |
| MHC Class II: Complementation group D | RFXAP | I |
| Monocyte chemoattractant protein 1 | MCP1 | I |
| Myeloid leukemia factor-1 | MLF1 | I |
| Myeloperoxidase | MPO | I |
| N-acyl hydrolase | | I |
| NADPH oxidase | | I |
| Natural resistance-associated macrophage | NRAMP1 | I |
| protein 1 | | |
| NB6 | | I |
| Neuronal apoptosis inhibitory protein | NAIP | I |
| Neuronal molecule-1 | | I |
| Neuronal molecule-1 receptor | | I |
| Neutrophil cystolic factor 1 | NCF1 | I |
| Neutrophil cystolic factor 2 | NCF2 | I |
| Nuclear factor 1-kappa-B-like gene | IKBL | I |
| Nuclear factor kappa beta | NFKB | I |
| Peanut-like 1 | PNUTL1 | I |
| Phagocytin | | I |
| Phospholipase A2, group 10 | PLA2G10 | I |
| Phospholipase A2, group 1B | PLA2G1B | I |
| Phospholipase A2, group 2A | PLA2G2A | I |
| Phospholipase A2, group 2B | PLA2G2B | I |
| Phospholipase A2, group 4A | PLA2G4A | I |
| Phospholipase A2, group 4C | PLA2G4C | I |
| Phospholipase A2, group 5 | PLA2G5 | I |
| Phospholipase A2, group 6 | PLA2G6 | I |
| Phospholipase C alpha | | I |
| Phospholipase C beta | | I |
| Phospholipase C delta | PLCD1 | I |
| Phospholipase C epsilon | | I |
| Phospholipase C gamma | PLCG1 | I |
| Platelet glycoprotein 1b, alpha | GP1BA | I |
| Platelet glycoprotein 1b, beta | GP1BB | I |
| Platelet glycoprotein 1b, gamma | GP1BG | I |
| Platelet glycoprotein IX | GP9 | I |
| Platelet glycoprotein V | GP5 | I |
| Platelet-activating factor | PAFAH1B1 or | I |
| acetylhydrolase 1B | LIS1 | |
| Platelet-activating factor acetylhydrolase 2 | PAFAH2 | I |
| Platelet-activating factor receptor | PAFR | I |
| Poliovirus receptor | PVR, PVS | I |
| Prekallikrein | | I |
| Properdin P factor, complement | PFC, PFD | I |
| Prostacyclin synthase | | I |
| Prostaglandin 15-OH dehydrogenase | HGPD; PGDH | I |
| Prostaglandin D—DP receptor | | I |
| Prostaglandin E1 receptor | | I |
| Prostaglandin E2 receptor | | I |
| Prostaglandin E3 receptor | | I |
| Prostaglandin F—FP receptor | | I |
| Prostaglandin F2 alpha receptor | | I |
| Prostaglandin IP receptor | | I |
| Protein C | PROC | I |
| Protein C inhibitor | PCI | I |
| Protein S | PROS1 | I |
| Proteinase 3 | | I |
| Prothrombin precursor | F2 | I |
| SAP (SLAM-associated protein) | SH2D1A | I |
| Severe combined immunodeficiency, type | SC1DA | I |
| A (Athabascan) | | |
| Signaling lymphocyte activation molecule | SLAM | I |
| Sjoegren (Sjogren) syndrome antigen A1 | SSA1 | I |
| SYK-related tyrosine kinase | SRK | I |
| T-cell acute lymphocytic leukemia 1 | TAL1 | I |
| T-cell acute lymphocytic leukemia 2 | TAL2 | I |
| T-cell receptor, alpha | TCRA | I |
| T-cell receptor, delta | TCRD | I |
| Terminal deoxynucleotidyltransferase | TDT | I |
| Thrombin receptor | F2R | I |
| Thrombomodulin | THBD | I |
| Thromboxane A synthase 1 | TBXAS1 | I |
| Thromboxane A2 | TXA2 | I |
| Thromboxane A2 receptor | TBXA2R | I |
| Thy-1 T-cell antigen | THY1 | I |
| Thymic humoral factor | | I |
| Thymosin | | I |
| Tip-associated protein | TAP | I |
| Toll-like receptor 4 | TLR4 | I |
| Tumour necrosis factor (TNF) receptor | TRAF1 | I |
| associated | | |
| factor 1 | | |
| Tumour necrosis factor (TNF) receptor | TRAF2 | I |
| associated | | |
| factor 2 | | |
| Tumour necrosis factor (TNF) receptor | TRAF3 | I |
| associated | | |
| factor 3 | | |
| Tumour necrosis factor (TNF) receptor | TRAF4 | I |
| associated | | |
| factor 4 | | |
| Tumour necrosis factor (TNF) receptor | TRAF5 | I |
| associated | | |
| factor 5 | | |
| Tumour necrosis factor (TNF) receptor | TRAF6 | I |
| associated | | |
| factor 6 | | |
| Tumour necrosis factor alpha | TNFA | I |
| Tumour necrosis factor alpha receptor | TNFAR | I |
| Tumour necrosis factor beta | TNFB | I |
| Tumour necrosis factor beta receptor | TNFBR | I |
| Tumour suppresssor gene DRA | DRA | I |
| Uridine monophosphate kinase | UMPK | I |
| Uridine monophosphate synthetase | UMPS | I |
| Vimentin | VIM | I |
| Wiskott-Aldrich syndrome protein | WASP, THC | I |
| 17-ketosteroid reductase | | N |
| Acetylcholine receptor, nicotinic, | CHRNA1 | N |
| alpha A1 | | |
| Acetylcholine receptor, nicotinic, | CHRNA2 | N |
| alpha A2 | | |
| Acetylcholine receptor, nicotinic, | CHRNA3 | N |
| alpha A3 | | |
| Acetylcholine receptor, nicotinic, | CHRNA4 | N |
| alpha A4 | | |
| Acetylcholine receptor, nicotinic, | CHRNA5 | N |
| alpha A5 | | |
| Acetylcholine receptor, nicotinic, | CHRNA6 | N |
| alpha A6 | | |
| Acetylcholine receptor, nicotinic, | CHRNA7 | N |
| alpha A7 | | |
| Acetylcholine receptor, nicotinic, beta 1 | CHRNB1 | N |
| Acetylcholine receptor, nicotinic, beta 2 | CHRNB2 | N |
| Acetylcholine receptor, nicotinic, beta 3 | CHRNB3 | N |
| Acetylcholine receptor, nicotinic, beta 4 | CHRNB4 | N |
| Acetylcholine receptor, nicotinic, epsilon | CHRNE | N |
| Acetylcholine receptor, nicotinic, gamma | CHRNG | N |
| Adenosine receptor A1 | ADORA1 | N |
| Adenosine receptor A2A | ADORA2A | N |
| Adenosine receptor A2B | ADORA2B | N |
| Adenosine receptor A3 | ADORA3 | N |
| Adenyl cyclase | | N |
| Adrenergic receptor, alpha1 | ADRA1 | N |
| Adrenergic receptor, alpha2 | ADRA2 | N |
| Adrenergic receptor, beta1 | ADRB1 | N |
| Adrenergic receptor, beta2 | ADRB2 | N |
| Adrenergic receptor, beta3 | ADRB3 | N |
| alpha thalassemia gene | ATRX | N |
| alpha-synuclein | SNCA | N |
| Amyloid beta (A4) precursor | APBB1 | N |
| protein-binding, APBB 1 | | |
| Amyloid beta A4 precursor protein | APP | N |
| Amyloid beta A4 precursor-like protein | APLP | N |
| Arginine vasopressin | AVP | N |
| Arginine vasopressin receptor 1A | AVPR1A | N |
| Arginine vasopressin receptor 1B | AVPR1B | N |
| Arginine vasopressin receptor 2 | AVPR2 | N |
| Aspartate receptor | | N |
| Benzodiazepine receptor | | N |
| beta-endorphin receptor | | N |
| beta-synuclein | SNCB | N |
| Calcitonin receptor/Calcitonin gene-related | CALCR | N |
| peptide | | |
| receptor | | |
| Calcitonin/Calcitonin gene-related peptide | CALCA | N |
| alpha | | |
| Calcium channel, voltage-dependent, | CACNA1F | N |
| alpha 1F subunit | | |
| Calcium channel, voltage-dependent, | CACNA1B | N |
| Alpha-B (CACNL1A5) | | |
| Calcium channel, voltage-dependent, | CACNA1C | N |
| Alpha-1C | | |
| Calcium channel, voltage-dependent, | CACNA1D | N |
| Alpha-1D | | |
| Calcium channel, voltage-dependent, | CACNA1E | N |
| Alpha-1E (CACNL1A6) | | |
| Calcium channel, voltage-dependent, | CACNA2 | N |
| Alpha-2/delta | | |
| Calcium channel, voltage-dependent, | CACNB1 | N |
| Beta 1 | | |
| Calcium channel, voltage-dependent, | CACNB3 | N |
| Beta 3 | | |
| Calcium channel, voltage-dependent, | CACNA1S | N |
| L type, alpha 1S subunit | | |
| Calcium channel, voltage-dependent, | CACNG2 | N |
| Neuronal, Gamma | | |
| Calcium channel, voltage-dependent, | CACNA1A | N |
| P/Q type, alpha 1A subunit | | |
| Calcium channel, voltage-dependent, | | N |
| T-type | | |
| Calretinin | CALB2 | N |
| Cannabinoid receptor | CNR1 | N |
| Carnosinase | | N |
| Cartilage oligomeric matrix protein | COMP, EDM1, | N |
| | PSACH | |
| Cartilage-hair hypoplasia gene | CHH | N |
| Cellubrevin | CEB | N |
| Ceroid lipofuscinosis neuronal 2 | CLN2 | N |
| Ceroid lipofuscinosis neuronal 3 | CLN3 | N |
| Ceroid lipofuscinosis neuronal 4 | CLN4 | N |
| Ceroid lipofuscinosis neuronal 5 | CLN5 | N |
| Ceroid lipofuscinosis neuronal 6 | CLN6 | N |
| Cholecystokinin | CCK | N |
| Cholecystokinin B receptor | CCKBR | N |
| Corticosteroid binding globulin | CBG | N |
| Cyclic nucleotide gated channel alpha 1, | CNGA1 | N |
| CNGA1 | | |
| Cyclic nucleotide gated channel alpha 3, | CNGA3 | N |
| CNGA3 | | |
| Cystic fibrosis transmembrane | CFTR | N |
| conductance regulator, CFTR | | |
| Deafness autosomal dominant 5 | DFNA5 | N |
| Deafness dystonia peptide | DDP | N |
| Diaphanous 1 | DIAPH1 | N |
| Diaphanous 2 | DIAPH2 | N |
| Dihydrolipoamide branched chain | DBT | N |
| transacylase | | |
| Dihydrolipoamide dehydrogenase | DLD | N |
| Dihydrolipoamide succinyltransferase | | N |
| Dopamine receptors D1 | DRD1 | N |
| Dopamine receptors D2 | DRD2 | N |
| Dopamine receptors D3 | DRD3 | N |
| Dopamine receptors D4 | DRD4 | N |
| Dopamine receptors D5 | DRD5 | N |
| Dynorphin receptor | | N |
| Endobrevin | VAMP8 | N |
| Endothelin 1 | EDN1 | N |
| Endothelin 2 | EDN2 | N |
| Endothelin 3 | EDN3 | N |
| Endothelin converting enzyme | ECE1 | N |
| Endothelin receptor type A | EDNRA | N |
| Endothelin receptor type B | EDNRB | N |
| Fragile site, folic acid type, rare, | FRAXA | N |
| fra(X) A | | |
| Fragile site, folic acid type, rare, | FRAXE | N |
| fra(X) E | | |
| Fragile site, folic acid type, rare, | FRAXF | N |
| fra(X) F | | |
| GABA receptor, alpha 1 | GABRA1 | N |
| GABA receptor, alpha 2 | GABRA2 | N |
| GABA receptor, alpha 3 | GABRA3 | N |
| GABA receptor, alpha 4 | GABRA4 | N |
| GABA receptor, alpha 5 | GABRA5 | N |
| GABA receptor, alpha 6 | GABRA6 | N |
| GABA receptor, beta 1 | GABRB1 | N |
| GABA receptor, beta 2 | GABRB2 | N |
| GABA receptor, beta 3 | GABRB3 | N |
| GABA receptor, gamma 1 | GABRG1 | N |
| GABA receptor, gamma 2 | GABRG2 | N |
| GABA receptor, gamma 3 | GABRG3 | N |
| Galanin | GAL | N |
| Galanin receptor | GALNR1 | N |
| Gephyrin | | N |
| Glial-cell derived neurotrophic factor | | N |
| (GDNF) receptor | | |
| Glial-cell derived neurotrophic factor, | GDNF | N |
| GDNF | | |
| Glutamate receptor 1 | GLUR1 | N |
| Glutamate receptor 2 | GLUR2 | N |
| Glutamate receptor 3 | GLUR3 | N |
| Glutamate receptor 4 | GLUR4 | N |
| Glutamate receptor 5 | GLUR5 | N |
| Glutamate receptor 6 | GLUR6 | N |
| Glutamate receptor 7 | GLUR7 | N |
| Glutamate receptor, ionotropic, | NMDAR1 | N |
| NMDA 1 | | |
| Glutamate receptor, ionotropic, | NMDAR2A | N |
| NMDA 2A | | |
| Glutamate receptor, ionotropic, | NMDAR2B | N |
| NMDA 2B | | |
| Glutamate receptor, ionotropic, | NMDAR2C | N |
| NMDA 2C | | |
| Glutamate receptor, ionotropic, | NMDAR2D | N |
| NMDA 2D | | |
| Glycine receptor, alpha | GLRA2 | N |
| Glycine receptor, beta | | N |
| Glycine transporter | GLYT | N |
| Guanine nucleotide-binding protein, alpha | GNA11 | N |
| inhibiting activity polypeptide 1, GNA11 | | |
| Guanine nucleotide-binding protein, alpha | GNA12 | N |
| inhibiting activity polypeptide 2, GNA12 | | |
| Guanine nucleotide-binding protein, alpha | GNA13 | N |
| inhibiting activity polypeptide 3, GNA13 | | |
| Guanine nucleotide-binding protein, alpha | GNAS1 | N |
| stimulating activity polypeptide, GNAS1 | | |
| Guanine nucleotide-binding protein, alpha | GNAS2 | N |
| stimulating activity polypeptide, GNAS2 | | |
| Guanine nucleotide-binding protein, alpha | GNAS3 | N |
| stimulating activity polypeptide, GNAS3 | | |
| Guanine nucleotide-binding protein, alpha | GNAS4 | N |
| stimulating activity polypeptide, GNAS4 | | |
| Guanine nucleotide-binding protein, alpha | GNAT1 | N |
| transducing activity polypeptide, GNAT1 | | |
| Guanine nucleotide-binding protein, alpha | GNAT2 | N |
| transducing activity polypeptide, GNAT2 | | |
| Guanine nucleotide-binding protein, alpha | GNAO1 | N |
| activating activity polypeptide, GNAO | | |
| Guanine nucleotide-binding protein, beta | GNB3 | N |
| polypeptide 3 | | |
| Guanine nucleotide-binding protein, | GNG5 | N |
| gamma polypeptide 5 | | |
| Guanine nucleotide-binding protein, q | GNAQ | N |
| polypeptide | | |
| Gustducin, alpha (taste-specific G protein) | GDCA | N |
| H(+), K(+)—ATPase | ATP4B | N |
| Hippocampal cholinergic neurostimulating | HCNP | N |
| peptide, | | |
| Histamine receptors, H1 | | N |
| Histamine receptors, H2 | | N |
| Histamine receptors, H3 | | N |
| Inositol monophosphatase | IMPA1 | N |
| Inositol polyphosphate 1-phosphatase | INPP1 | N |
| Islet amyloid polypeptide | IAPP | N |
| L1 cell adhesion molecule | L1CAM | N |
| Luteinizing hormone-releasing hormone | | N |
| Luteinizing hormone-releasing hormone | | N |
| receptor | | |
| Melatonin receptor 1A | MTNR1A | N |
| Melatonin receptor 1B | MTNR1B | N |
| Muscarinic receptor, M1 | CHRM1 | N |
| Muscarinic receptor, M2 | CHRM2 | N |
| Muscarinic receptor, M3 | CHRM3 | N |
| Muscarinic receptor, M4 | CHRM4 | N |
| Muscarinic receptor, M5 | CHRM5 | N |
| Neurexin | | N |
| Neurite growth-promoting factor 2 | MDK | N |
| Neurite inhibitory protein | | N |
| Neurokinin A | NKNA | N |
| Neurokinin B | NKNB | N |
| Neuropeptide Y | NPY | N |
| Neuropeptide Y receptor Y1 | NPY1R | N |
| Neuropeptide Y receptor Y2 | NPY2R | N |
| Neurotensin | NTS | N |
| Neurotensin receptor | NTSR1 | N |
| Opioid receptor, delta | OPRD1 | N |
| Opioid receptor, kappa | OPRK1 | N |
| Opioid receptor, mu | OPRM1 | N |
| Otoferlin | OTOF | N |
| Oxytocin | OXT | N |
| Oxytocin receptor | OXTR | N |
| Parkin | PARK2 | N |
| Pituitary adenylate cyclase activating | PACAP | N |
| peptide | | |
| Pituitary adenylate cyclase activating | PACAP1R | N |
| peptide receptor | | |
| Postsynaptic density-95 protein | PSD95 | N |
| Potassium inwardly-rectifying channel J1 | KCNJ1 | N |
| Potassium inwardly-rectifying channel J11 | KCNJ11 | N |
| Potassium voltage-gated channel A1 | KCNA1 | N |
| Potassium voltage-gated channel E1 | KCNE1 | N |
| Potassium voltage-gated channel Q1 | KCNQ1 | N |
| Potassium voltage-gated channel Q2 | KCNQ2 | N |
| Potassium voltage-gated channel Q3 | KCNQ3 | N |
| Potassium voltage-gated channel Q4 | KCNQ4 | N |
| Potassium channel, subfamily K, | KCNK1 | N |
| member 1 | | |
| Potassium channel, subfamily K, | KCNK2 | N |
| member 2 | | |
| Potassium channel, subfamily K, | KCNK3 | N |
| member 3 | | |
| Potassium channel, calcium-activated, | KCNN4 | N |
| Preproenkephalin | PENK | N |
| Prion protein | PRNP | N |
| Prodynorphin | | N |
| Proopiomelanocortin | POMC | N |
| Prosaposin | PSAP | N |
| Proteolipid protein | PLP | N |
| Purinergic receptor P1A1 | | N |
| Purinergic receptor P1A2 | | N |
| Purinergic receptor P1A3 | | N |
| Purinergic receptor P2X, 1 | P2RX1 | N |
| Purinergic receptor P2X, 2 | P2RX2 | N |
| Purinergic receptor P2X, 3 | P2RX3 | N |
| Purinergic receptor P2X, 4 | P2RX4 | N |
| Purinergic receptor P2X, 5 | P2RX5 | N |
| Purinergic receptor P2X, 6 | P2RX6 | N |
| Purinergic receptor P2X, 7 | P2RX7 | N |
| Purinergic receptor P2Y, 1 | P2RX1 | N |
| Purinergic receptor P2Y, 2 | P2RY2 | N |
| Purinergic receptor P2Y, 11 | P2RY11 | N |
| Rabphilin | | N |
| RAS-associated protein, RAB3A | RAB3A | N |
| Rim | | N |
| S100 calcium-binding protein A1 | S100A1 | N |
| S100 calcium-binding protein A2 | S100A2 | N |
| S100 calcium-binding protein A3 | S100A3 | N |
| S100 calcium-binding protein A4 | S100A4 | N |
| S100 calcium-binding protein A5 | S100A5 | N |
| S100 calcium-binding protein A6 | S100A6 | N |
| S100 calcium-binding protein A7 | S100A7 | N |
| S100 calcium-binding protein A8 | S100A8 | N |
| S100 calcium-binding protein A9 | S100A9 | N |
| S100 calcium-binding protein B | S100B | N |
| S100 calcium-binding protein P | S100P | N |
| Secretase, alpha | | N |
| Secretase, alpha | | N |
| Secretase, beta | | N |
| Secretase, gamma | | N |
| Selectin E | SELE | N |
| Selectin L | SELL | N |
| Selectin P | SELP | N |
| Serotonin receptor, 5HT1A | HTR1A | N |
| Serotonin receptor, 5HT1B | HTR1B | N |
| Serotonin receptor, 5HT1C | HTR1C | N |
| Serotonin receptor, 5HT1D | HTR1D | N |
| Serotonin receptor, 5HT1E | HTR1E | N |
| Serotonin receptor, 5HT1F | HTR1F | N |
| Serotonin receptor, 5HT2A | HTR2A | N |
| Serotonin receptor, 5HT2B | HTR2B | N |
| Serotonin receptor, 5HT2C | HTR2C | N |
| Serotonin receptor, 5HT3 | HTR3 | N |
| Serotonin receptor, 5HT4 | HTR4 | N |
| Serotonin receptor, 5HT5 | HTR5 | N |
| Serotonin receptor, 5HT6 | HTR6 | N |
| Serotonin receptor, 5HT7 | HTR7 | N |
| Sodium channel, non-voltage gated 1, | SCNN1A | N |
| alpha | | |
| Sodium channel, non-voltage gated 1, | SCNN1B | N |
| beta | | |
| Sodium channel, non-voltage gated 1, | SCNN1G | N |
| gamma | | |
| Sodium channel, voltage gated, type IV, | SCN4A | N |
| alpha polypeptide | | |
| Sodium channel, voltage gated, type V, | SCN5A | N |
| alpha polypeptide | | |
| Sodium channel, voltage-gated, type 1, | SCN1B | N |
| beta polypeptide | | |
| Somatostatin | SST | N |
| Somatostatin receptor, SSTR1 | SSTR1 | N |
| Somatostatin receptor, SSTR2 | SSTR2 | G |
| Somatostatin receptor, SSTR3 | SSTR3 | N |
| Somatostatin receptor, SSTR4 | SSTR4 | N |
| Somatostatin receptor, SSTR5 | SSTR5 | N |
| Spinocerebellar ataxia 8 gene | SCA8 | N |
| Substance P | | N |
| Synapsin 1a & 1b | SYN1 | N |
| Synapsin 2a & 2b | SYN2 | N |
| Synaptic vesicle amine transporter | SVAT | N |
| Synaptic vesicle protein 2 | SV2 | N |
| Synaptobrevin 1 | SYB1 | N |
| Synaptobrevin 2 | SYB2 | N |
| Synaptogyrin | | N |
| Synaptophysin | SYP | N |
| Synaptosomal-associated protein, 25KD | SNAP25 | N |
| Synaptotagmin 1 | SYT1 | N |
| Synaptotagmin 2 | SYT2 | N |
| Syntaxin 1 | STX1 | N |
| Tachykinin receptor, NK1R | TACR1 | N |
| Tachykinin receptor, NK2R | TACR2 | N |
| Tachykinin receptor, NK3R | TACR3 | N |
| Thyrotropin releasing hormone | TRH | N |
| Thyrotropin releasing hormone receptor | TRHR | N |
| Transcription factor, TUPLE1 | TUPLE1 | N |
| Tremor, essential 1 | ETM1 | N |
| Tremor, essential 2 | ETM2 | N |
| Tryptophan 2,3-dioxygenase | TDO2 | N |
| Vacuolar proton pump, subunit 1 | VPP1 | N |
| Vacuolar proton pump, subunit 3 | VPP3 | N |
| Vasoactive intestinal polypeptide | VIP | N |
| Vasoactive intestinal polypeptide receptor | VIPR | N |
| Vesicular monoamine transporter 1 | VMAT1 | N |
| Vesicular monoamine transporter 2 | VMAT2 | N |
| Absent in melanoma 1 gene | AIM1 G | |
| Acrosin | ACR | G |
| Activin | | G |
| Activin A receptor, type 2-like kinase 1 | ACVRL1 | G |
| Activin A receptor, type 2B | ACVR2B | G |
| Adenomatous polyposis coli tumour | APC | G |
| supressor gene | | |
| Adrenocorticotrophic hormnone (ACTH) | ACTHR | G |
| receptor | | |
| Aldosterone receptor | MLR | G |
| Alkaptonuria gene | AKU | G |
| alpha tectorin | TECTA | G |
| alpha-actinin 2 | ACTN2 | G |
| alpha-actinin 3 | ACTN3 | G |
| Alpha-fetoprotein | AFP | G |
| Amphiregulin | AREG | G |
| Androgen receptor | AR | G |
| Angiopoietin 1 | ANGPT1 | G |
| Angiopoietin 2 | ANGPT2 | G |
| Anti-Mullerian hormone | AMH | G |
| Anti-Mullerian hormone type 2 receptor | AMHR2 | G |
| AP-2, alpha | TFAP2A | G |
| AP-2, beta | TFAP2B | G |
| AP-2, gamma | TFAP2C | G |
| Apical protein, xenopus laevis-like | APXL | G |
| Apopain | CPP32 | G |
| Archaete-scute homolog 1 | ASH1 | G |
| Archaete-scute homolog 2 | ASH2 | G |
| Astrotactin | ASTN | G |
| Ataxia telangiectasia complementation | ATD, ATDC | G |
| group D | | |
| Ataxia telangiectasia gene, AT | ATM | G |
| Ataxin 1 | SCA1 | G |
| Ataxin 2 | SCA2 | G |
| Ataxin 3 | MJD | G |
| Atrial natriuretic peptide | ANP | G |
| Atrial natriuretic peptide receptor A | NPR1 | G |
| Atrial natriuretic peptide receptor B | NPR2 | G |
| Atrial natriuretic peptide receptor C | NPR3 | G |
| Atrophin 1 | DRPLA | G |
| Azoospermia factor 1 | AZF1 | G |
| Bagpipe homeobox, drosophila | BAPX1 | G |
| homolog of, 1 | | |
| BCL2-associated X protein | BAX | G |
| BCL2-related protein A1 | BCL2A1 | G |
| Beckwith-Wiedemann region 1A | BWR1A | G |
| Bloom syndrome protein | BLM | G |
| Bone morphogenetic protein, BMP1 | BMP1 | G |
| Bone morphogenetic protein, BMP2 | BMP2 | G |
| Bone morphogenetic protein, BMP3 | BMP3 | G |
| Bone morphogenetic protein, BMP4 | BMP4 | G |
| Bone morphogenetic protein, BMP5 | BMP5 | G |
| Bone morphogenetic protein, BMP6 | BMP6 | G |
| Bone morphogenetic protein, BMP7 | BMP7 | G |
| Bone morphogenetic protein, BMP8 | BMP8 | G |
| Brain derived neurotrophic factor | BDNF | G |
| Brain derived neurotrophic factor (BDNF) | BDNFR | G |
| receptor | | |
| BRCA1-associated RING domain gene 1 | BARD1 | G |
| Breakpoint cluster region | BCR | G |
| Breast cancer 1 | BRCA1 | G |
| Breast cancer 2 | BRCA2 | G |
| Breast cancer, ductal, 1 | BRCD1 | G |
| Breast cancer, ductal, 2 | BRCD2 | G |
| Bruton agammaglobulinaemia tyrosine | BTK | G |
| kinase | | |
| Cadherin E | CDH1 | G |
| Cadherin EP | | G |
| Cadherin N | CDH2 | G |
| Cadherin P | CDH3 | G |
| Calbindin 1 | CALB1 | G |
| Calbindin D9K | CALB3 | G |
| Calmodulin 1 | CALM1 | G |
| Calmodulin 2 | CALM2 | G |
| Calmodulin 3 | CALM3 | G |
| Calmodulin-dependant protein kinase II | CAMK2A | G |
| Calnexin | CANX | G |
| Cardiac-specific homeobox, CSX | CSX | G |
| Caspase 1 | CASP1 | G |
| Caspase 10 | CASP10 | G |
| Caspase 2 | CASP2 | G |
| Caspase 3 | CASP3 | G |
| Caspase 4 | CASP4 | G |
| Caspase 5 | CASP5 | G |
| Caspase 6 | CASP6 | G |
| Caspase 7 | CASP7 | G |
| Caspase 8 | CASP8 | G |
| Caspase 9 | CASP9 | G |
| Catenin, alpha | CTNNA1 | G |
| Catenin, beta | CTNNB1 | G |
| Catenin, gamma | | G |
| Cdc 25 phosphatase | | G |
| Cdc2 | CDC2 | G |
| CDX1 | | G |
| CEA | | G |
| Cell adhesion molecule, intercellular, | ICAM1 | G |
| ICAM | | |
| Cell adhesion molecule, leukocyte- | LECAM1 | G |
| endothelial, LECAM (CD62) | | |
| Cell adhesion molecule, liver, LCAM | LCAM | G |
| Cell adhesion molecule, neural, NCAM1 | NCAM1 | G |
| Cell adhesion molecule, neural, | NCAM120 | G |
| NCAM120 | | |
| Cell adhesion molecule, neural, NCAM2 | NCAM2 | G |
| Cell adhesion molecule, platelet- | PACAM1 | G |
| endothelial, PECAM | | |
| Cell adhesion molecule, vascular, VCAM | VCAM1 | G |
| c-erbB1 | ERBB1 | G |
| c-erbB2 | ERBB2 | G |
| c-erbB3 | ERBB3 | G |
| c-erbB4 | ERBB4 | G |
| Cholestasis, progressive familial | FIC1 | G |
| intrahepatic 1 gene | | |
| Chromogranin A | CHGA | G |
| Ciliary neurotrophic factor (CNTF) | CNTF | G |
| Ciliary neurotrophic factor (CNTF) | CNTFR | G |
| receptor | | |
| c-kit receptor tyrosine kinase | | G |
| Cleavage signal-1 protein | CS1 | G |
| Cleft palate gene | CPX | G |
| Clusterin | CLU | G |
| Cockayne syndrome gene, CKN1 | CKN1 | G |
| Collapsin | | G |
| Colony-stimulating factor 1 | CSF1 | G |
| Colony-stimulating factor 1 receptor | CSF1R | G |
| Colony-stimulating factor 2 | CSF2 | G |
| Colony-stimulating factor 2 alpha receptor | CSF2RA | G |
| Colony-stimulating factor 2 beta receptor | CSF2RB | G |
| Colony-stimulating factor 3 | CSF3 | G |
| Colony-stimulating factor 3 receptor | CSF3R | G |
| Cone-rod homeobox-containing gene | CRX | G |
| Contactin | CNTN1 | G |
| Core-binding factor, alpha 1 | CBFA1 | G |
| Core-binding factor, alpha 2 | CBFA2 | G |
| Core-binding factor, beta | CBFB | G |
| Creb binding protein | CREBBP | G |
| c-src tyrosine kinase | CSK | G |
| Cyclic AMP response element binding | CREB | G |
| protein | | |
| Cyclic AMP response element modulator | CREM | G |
| Cyclic AMP-dependent protein kinase | PKA | G |
| Cyclin A | CCNA | G |
| Cyclin B | CCNB | G |
| Cyclin C | CCNC | G |
| Cyclin D | CCND1 | G |
| Cyclin E | CCNE | G |
| Cyclin F | CCNF | G |
| Cyclin-dependent kinase 1 | CDK1 | G |
| Cyclin-dependent kinase 10 | CDK10 | G |
| Cyclin-dependent kinase 2 | CDK2 | G |
| Cyclin-dependent kinase 3 | CDK3 | G |
| Cyclin-dependent kinase 4 | CDK4 | G |
| Cyclin-dependent kinase 5 | CDK5 | G |
| Cyclin-dependent kinase 6 | CDK6 | G |
| Cyclin-dependent kinase 7 | CDK7 | G |
| Cyclin-dependent kinase 8 | CDK8 | G |
| Cyclin-dependent kinase 9 | CDK9 | G |
| Cyclin-dependent kinase inhibitor 1A | CDKN1A | G |
| (P21, CIP1) | | |
| Cyclin-dependent kinase inhibitor 1B | CDKN1B | G |
| (P27, KIP1) | | |
| Cyclin-dependent kinase inhibitor 1C | CDKN1C | G |
| (P57, KIP2) | | |
| Cyclin-dependent kinase inhibitor 2A | CDKN2A | G |
| (p16) | | |
| Cyclin-dependent kinase inhibitor 3 | CDKN3 | G |
| Defender against cell death 1 | DAD1 | G |
| Deleted in azoospermia | DAZ | G |
| Deleted in colorectal carcinoma | DCC | G |
| Deleted in malignant brain tumours 1 | DMBT1 | G |
| Dentin sialophosphoprotein | DSPP | G |
| Desert hedgehog, dhh | | G |
| Disrupted meiotic cDNA 1, homolog | DMC1 | G |
| Distal-less homeobox 1 | DLX1 | G |
| Distal-less homeobox 2 | DLX2 | G |
| Distal-less homeobox 3 | DLX3 | G |
| Distal-less homeobox 4 | DLX4 | G |
| Distal-less homeobox 5 | DLX5 | G |
| Distal-less homeobox 6 | DLX6 | G |
| Dynamin | DNM1 | G |
| Dynein | | G |
| E74-like factor 1, ELF1 | ELF1 | G |
| EB1 | | G |
| Empty spiracles (drosophila) homologue 1 | EMX1 | G |
| Empty spiracles (drosophila) homologue 2 | EMX2 | G |
| Endometrial bleeding-associated factor | EBAF | G |
| Engrailed-1 | EN1 | G |
| Engrailed-2 | EN2 | G |
| Ephrin receptor tyrosine kinase A | EPHA | G |
| Ephrin receptor tyrosine kinase B | EPHB | G |
| Ephrin-A | EFNA | G |
| Ephrin-B | EFNB | G |
| Epidermal growth factor | EGF | G |
| Epidermal growth factor receptor | EGFR | G |
| Erythroid kruppel-like factor | EKLF | G |
| Estrogen receptor | ESR | G |
| Eukaryotic initiation translation factor | EIF4E | G |
| EWS RNA-binding protein | EWSR1 | G |
| Eyes absent 1 | EYA1 | G |
| Eyes absent 2 | EYA2 | G |
| Eyes absent 3 | EYA3 | G |
| Fc fragment of IgG, high affinity IA, | FCGR1A | G |
| receptor for | | |
| Fc fragment of IgG, low affinity IIa, | FCGR2A | G |
| receptor for (CD32) | | |
| Fc fragment of IgG, low affinity IIIa, | FCGR3A | G |
| receptor for (CD 16) | | |
| Fertilin protein | FTNB | G |
| Fibrillin 1 | FBN1 | G |
| Fibrillin 2 | FBN2 | G |
| Fibroblast growth factor | FGF1 | G |
| Fibroblast growth factor receptor 1 | FGFR1 | G |
| Fibroblast growth factor receptor 2 | FGFR2 | G |
| Fibroblast growth factor receptor 3 | FGFR3 | G |
| Fibronectin precursor | FNI | G |
| Flightless-II, Drosophila homolog of | FLII | G |
| Folic acid receptor | FOLR | G |
| Follicle stimulating hormone receptor | FSHR, ODG1 | G |
| Follicle stimulating hormone, FSH | FSHB | G |
| Follistatin | | G |
| Forkhead rhabdomyosarcoma gene | FKHR | G |
| Forkhead transcription factor 10 | FKHL10 | G |
| Forkhead transcription factor 14 | FKHL14 | G |
| Forkhead transcription factor 7 | FKHL7 | G |
| Frataxin | FRDA | G |
| Fringe secreted protein, lunatic | LFNG | G |
| Fringe secreted protein, manic | MFNG | G |
| Fringe secreted protein, radical | RFNG | G |
| Fukuyama type congenital muscular | FCMD | G |
| dystrophy | | |
| G/T mismatch binding protein | GTBP, MSH6 | G |
| Galactosyltransferase 1 | GT1 | G |
| Galactosyltransferase, alpha 1,3 | GGTA1 | G |
| Galactosyltransferase, beta 3 | B3GALT | G |
| Gastrin | GAS | G |
| Gastrulation brain homeobox 2 | GBX2 | G |
| GDP dissociation inhibitor 1 | GDI1 | G |
| Gelsolin | GSN | G |
| Geniospasm 1 | GSM1 | G |
| Glioma chloride ion channel, GCC | | G |
| Glucagon receptor | GCGR | G |
| Glucagon-like peptide receptor 1 | GLP1R | G |
| Glucocorticoid receptor | GRL | G |
| Glypican 3 | GPC3, SDYS | G |
| Gonadotropin releasing hormone | GNRH | G |
| Gonadotropin releasing hormone receptor | GNRHR | G |
| Goosecoid GSC | | G |
| Growth arrest-specific homeobox | GAX | G |
| Growth factor receptor-bound protein 2 | GRB2 | G |
| Growth hormone 1 | GH1 | G |
| Growth hormone 2 (placental) | GH2 | G |
| Growth hormone receptor | GHR | G |
| Growth hormone releasing hormone | GHRH | G |
| (GHRH) | | |
| Growth hormone releasing hormone | GHRHR | G |
| receptor | | |
| Growth/differentiation factor 5 | GDF5 | G |
| GTP cylcohydrolase 1 | GCH1 | G |
| GTPase-activating protein, GAP | RASA1 | G |
| Hairless | HR | G |
| Hela tumor suppression gene | HTS1 | G |
| Heparin binding epidermal growth factor | HBEGF | G |
| Hepatocyte growth factor | HGF | G |
| High mobility group protein 1 | HMG1 | G |
| High mobility group protein 2 | HMG2 | G |
| High mobility group protein C | HMG1C | G |
| High mobility group protein Y | HMG1Y | G |
| Histone family H1 | H1 | G |
| Histone family H2 | H2 | G |
| Histone family H3 | H3 | G |
| Histone family H4 | H4 | G |
| HLH transcription factor HAND1 | HAND1 | G |
| HLH transcription factor HAND2 | HAND2 | G |
| Holoprosencephaly 1 | HPE1 | G |
| Holoprosencephaly 2 | HPE2 | G |
| Holoprosencephaly 3 | HPE3 | G |
| Holoprosencephaly 4 | HPE4 | G |
| Homeobox (HOX) gene A1 | HOXA1 | G |
| Homeobox (HOX) gene A2 | HOXA2 | G |
| Homeobox (HOX) gene A3 | HOXA3 | G |
| Homeobox (HOX) gene A4 | HOXA4 | G |
| Homeobox (HOX) gene A5 | HOXA5 | G |
| Homeobox (HOX) gene A6 | HOXA6 | G |
| Homeobox (HOX) gene A7 | HOXA7 | G |
| Homeobox (HOX) gene A8 | HOXA8 | G |
| Homeobox (HOX) gene A9 | HOXA9 | G |
| Homeobox (HOX) gene A10 | HOXA10 | G |
| Homeobox (HOX) gene A11 | HOXA11 | G |
| Homeobox (HOX) gene A12 | HOXA12 | G |
| Homeobox (HOX) gene A13 | HOXA13 | G |
| Homeobox (HOX) gene B1 | HOXB1 | G |
| Homeobox (HOX) gene B2 | HOXB2 | G |
| Homeobox (HOX) gene B3 | HOXB3 | G |
| Homeobox (HOX) gene B4 | HOXB4 | G |
| Homeobox (HOX) gene B5 | HOXB5 | G |
| Homeobox (HOX) gene B6 | HOXB6 | G |
| Homeobox (HOX) gene B7 | HOXB7 | G |
| Homeobox (BOX) gene B8 | HOXB8 | G |
| Homeobox (HOX) gene B9 | HOXB9 | G |
| Homeobox (HOX) gene C4 | HOXC4 | G |
| Homeobox (HOX) gene C8 | HOXC8 | G |
| Homeobox (HOX) gene C9 | HOXC9 | G |
| Homeobox (HOX) gene C13 | HOXC13 | G |
| Homeobox (HOX) gene D1 | HOXD1 | G |
| Homeobox (HOX) gene D3 | HOXD3 | G |
| Homeobox (HOX) gene D4 | HOXD4 | G |
| Homeobox (HOX) gene D8 | HOXD8 | G |
| Homeobox (HOX) gene D9 | HOXD9 | G |
| Homeobox (HOX) gene D10 | HOXD10 | G |
| Homeobox (HOX) gene D12 | HOXD12 | G |
| Homeobox (HOX) gene D13 | HOXD13 | G |
| Homeobox 11 | HOX11 | G |
| Homeobox HB24 | HLX1 | G |
| Homeobox HB9 | HLXB9 | G |
| Homeobox, PROX1 | PROX1 | G |
| Human atonal gene | ATOH1 | G |
| Human chorionic gonadtrophin, hCG | CG | G |
| Human placental lactogen | CSH1 | G |
| Ikaros gene | IKAROS | G |
| Indian hedgehog, ihh | IHH | G |
| Inhibin, alpha | INHA | G |
| Inhibin, beta A | INHBA | G |
| Inhibin, beta B | INHBB | G |
| Inhibin, beta C | INHBC | G |
| Inositol 1,4,5-triphosphate receptor 1 | ITPR1 | G |
| Inositol 1,4,5-triphosphate receptor 3 | ITPR3 | G |
| Insulin | INS | G |
| Insulin promotor factor 1 | IPF1 | G |
| Insulin receptor | INSR | G |
| Insulin receptor substrate-1 | IRS1 | G |
| Insulin-like growth factor 1 | IGF1 | G |
| Insulin-like growth factor 1 receptor | IGF1R | G |
| Insulin-like growth factor 2 | IGF2 | G |
| Insulin-like growth factor 2 receptor | IGF2R | G |
| Integrin beta 1 | ITGB1 | G |
| Integrin beta 2 | ITGB2 | G |
| Integrin beta 3 | ITGB3 | G |
| Integrin beta 4 | ITGB4 | G |
| Integrin beta 5 | ITGB5 | G |
| Integrin beta 6 | ITGB6 | G |
| Integrin beta 7 | ITGB7 | G |
| Integrin, alpha 1 | ITGA1 | G |
| Integrin, alpha 2 | ITGA2 | G |
| Integrin, alpha 3 | ITGA3 | G |
| Integrin, alpha 4 | ITGA4 | G |
| Integrin, alpha 5 | ITGA5 | G |
| Integrin, alpha 6 | ITGA6 | G |
| Integrin, alpha 7 | ITGA7 | G |
| Integrin, alpha 8 | ITGA8 | G |
| Integrin, alpha 9 | ITGA9 | G |
| Integrin, alpha M | ITGAM | G |
| Integrin, alpha X | ITGAX | G |
| Janus kinase 1 | JAK1 | G |
| Janus kinase 2 | JAK2 | G |
| Janus kinase 3 | JAK3 | G |
| Kallman syndrome gene 1 | KAL1 | G |
| Kinectin | KTN1 | G |
| Kinesin, heavy chain | KNSL1 | G |
| Kinesin, light chain | KNS2 | G |
| Lamin A/C | LMNA | G |
| Laminin 5, alpha 3 | LAMA3 | G |
| Laminin 5, beta 3 | LAMB3 | G |
| Laminin 5, gamma 2 | LAMC2 | G |
| Laminin M | LAMM | G |
| Laminin receptor 1 | LAMR1 | G |
| Latent transforming growth factor-beta | LTBP2 | G |
| binding protein 2 | | |
| Leptin | LEP | G |
| Leptin receptor | LEPR | G |
| Leukaemia inhibitory factor | LIF | G |
| Leukaemia inhibitory factor receptor | LIFR | G |
| LH/choriogonadotropin (CG) receptor | LHCGR | G |
| LIM homeobox protein 1 | LHX1 | G |
| LIM homeobox protein 2 | LHX2 | G |
| LIM homeobox protein 3 | LHX3 | G |
| LIM homeobox protein 4 | LHX4 | G |
| LIM homeobox transcription factor 1, beta | LMX1B | G |
| Limb girdle muscular dystrophy 1A | LGMD1A | G |
| Limb girdle muscular dystrophy 1B | LGMD1B | G |
| Limb girdle muscular dystrophy 2G | LGMD2G | G |
| Limb girdle muscular dystrophy 2H | LGMD2H | G |
| Limbic associated membrane protein | LAMP | G |
| LIM-domain only protein 1 | LMO 1 | G |
| LIM-domain only protein 2 | LMO2 | G |
| LIM-domain only protein 3 | LMO3 | G |
| LIM-domain only protein 4 | LMO4 | G |
| Lipoma-preferred partner gene | LPP | G |
| Luteinizing hormone, beta chain | LHB | G |
| Lymphoid enhancer-binding factor | LEF-1 | G |
| Lysosome-associated membrane protein 1 | LAMP1 | G |
| Lysosome-associated membrane protein 2 | LAMP2 | G |
| MAD (mothers against decapentaplegic, | MADH2 | G |
| Drosophila) homologue 2 | | |
| MAD (mothers against decapentaplegic, | MADH3 | G |
| Drosophila) homologue 3 | | |
| MAD (mothers against decapentaplegic, | MADH4 | G |
| Drosophila) homologue 4 | | |
| MADS box transcription-enhancer | MEF2A | G |
| factor 2A | | |
| MADS box transcription-enhancer | MEF2B | G |
| factor 2B | | |
| MADS box transcription-enhancer | MEF2C | G |
| factor 2C | | |
| MADS box transcription-enhancer | MEF2D | G |
| factor 2D | | |
| MAPK kinase 1 | MAPKK1; | G |
| | MEK1 | |
| MAPK kinase 4 | MAPKK4; | G |
| | MEK4; SERK1 | |
| MAPK kinase 6 | MAPKK6; | G |
| | MEK6 | |
| MAPKK kinase | MAPKKK | G |
| Matrix Gla protein | MGP | G |
| MAX-interacting protein 1 | MXI1 | G |
| Menin | MEN1 | G |
| Mesoderm-specific transcript | MEST | G |
| Microphthalmia-associated transcription | MITF | G |
| factor | | |
| Midline 1 | MID1 | G |
| Mismatch repair gene, PMSL1 | PMS1 | G |
| Mismatch repair gene, PMSL2 | PMS2 | G |
| Mitogen-activated protein (MAP) kinase | MAPK | G |
| Motilin | MLN | G |
| Msh homeobox homolog 1 | MSX1 | G |
| Msh homeobox homolog 2 | MSX2 | G |
| Multidrug resistance associated protein | MRP | G |
| Mutated in colorectal cancers, MCC | MCC | G |
| MutL homolog 1 | MLH1 | G |
| MutS homolog 2 | MSH2 | G |
| MutS homolog 3 | MSH3 | G |
| Myelodysplasia syndrome 1 gene | MDS1 | G |
| Myogenic factor 3 | MYF3 | G |
| Myogenic factor 4 | MYF4 | G |
| Myogenic factor 5 | MYF5 | G |
| Na+, K+ ATPase, alpha | ATP1A1 | G |
| Na+, K+ ATPase, beta 1 | ATP1B1 | G |
| Na+, K+ ATPase, beta 2 | ATP1B2 | G |
| Na+, K+ ATPase, beta 3 | ATP1B3 | G |
| Necdin | NDN | G |
| Nerve growth factor | NGF | G |
| Nerve growth factor receptor | NGFR | G |
| Neural retina-specific gene | NRL | G |
| Neuregulin | HGL | G |
| Neurofibromin 1 | NF1 | G |
| Neurofibromin 2 | NF2 | G |
| Neurotrophic tyrosine kinase receptor 1 | NTRK1 | G |
| Neurotrophin 3 | NTF3 or NT3 | G |
| Neurturin | NRTN | G |
| Niacin receptor | | G |
| Nibrin | NBS1 | G |
| Nodal | NODAL | G |
| Noggin | NOG | G |
| Norrie disease protein | NDP | G |
| Notch 1 | NOTCH1 | G |
| Notch 2 | NOTCH2 | G |
| Notch 3 | NOTCH3 | G |
| Notch ligand—jagged 1 | JAG1, AGS | G |
| Nuclear factor of activated T cells (NFAT) | NFATC | G |
| complex, cytosolic | | |
| Nuclear factor of activated T cells (NFAT) | NFATP | G |
| complex, preexisting component | | |
| Nuclear mitotic apparatus protein 1 | NUMA 1 | G |
| Oligophrenin-1 | OPHN1 | G |
| Oncogene abl1 | abl1 | G |
| Oncogene abl2 | | G |
| Oncogene akt1 | | G |
| Oncogene akt2 | AKT2 | G |
| Oncogene axl | AXL | G |
| Oncogene bcl2 | | G |
| Oncogene bcr/abl | | G |
| Oncogene B-lym | | G |
| Oncogene B-raf | | G |
| Oncogene clk1 | | G |
| Oncogene c-myc | | G |
| Oncogene cot | | G |
| Oncogene crk | | G |
| Oncogene crk1 | | G |
| Oncogene ect2 | | G |
| Oncogene ELK1 | ELK1 | G |
| Oncogene ELK2 | ELK2 | G |
| Oncogene ems1 | | G |
| Oncogene ERB | | G |
| Oncogene ERB2 | | G |
| Oncogene ERBA | | G |
| Oncogene ERBAL2 | | G |
| Oncogene ERG (early reponse gene) | | G |
| Oncogene ETS1 | | G |
| Oncogene ETS2 | | G |
| Oncogene EVI1 | EVI1 | G |
| Oncogene fes | | G |
| Oncogene fgr | | G |
| Oncogene fos | FOS | G |
| Oncogene fps | | G |
| Oncogene GLI1 | GLI | G |
| Oncogene GLI2 | GLI2 | G |
| Oncogene GLI3 | GLI3 | G |
| Oncogene gro1 | | G |
| Oncogene gro2 | | G |
| Oncogene Ha-ras | HRAS | G |
| Oncogene hsl | | G |
| Oncogene hst | FGF4 | G |
| Oncogene int1 | WNT1 | G |
| Oncogene int2 | FGF3 | G |
| Oncogene int3 | Notch4 | G |
| Oncogene int4 | WNT3 | G |
| Oncogenejun | JUN | G |
| Oncogene KIT | KIT, PBT | G |
| Oncogene LCO | LCO | G |
| Oncogene l-myc | | G |
| Oncogene lpsa | | G |
| Oncogene lyn | | G |
| Oncogene maf | | G |
| Oncogene mas1 | | G |
| Oncogene mcf2 | | G |
| Oncogene mdm2 | MDM2 | G |
| Oncogene mel | | G |
| Oncogene met | MET | G |
| Oncogene mos | | G |
| Oncogene mpl | | G |
| Oncogene MUM1 | MUM1 | G |
| Oncogene myb | MYB | G |
| Oncogene myc | MYC | G |
| Oncogene n-myc | | G |
| Oncogene N-ras (neuroblastoma v-ras) | NRAS | G |
| Oncogene ovc | | G |
| Oncogene pim1 | | G |
| Oncogene pti-1sea | | G |
| Oncogene pvt1 | | G |
| Oncogene raf | RAF | G |
| Oncogene ralb | | G |
| Oncogene rel | | G |
| Oncogene ret | RET | G |
| Oncogene r-myc | | G |
| Oncogene ros | | G |
| Oncogene R-ras | | G |
| Oncogene sis | PDGFB | G |
| Oncogene ski | | G |
| Oncogene sno | | G |
| Oncogene spi1 | | G |
| Oncogene src | | G |
| Oncogene tc2l | | G |
| Oncogene TEL | ETV6 | G |
| Oncogene tim | | G |
| Oncogenc vavtrk | | G |
| Oncogene v-Ki-ras2 | KRAS2 | G |
| Oncogene yes | | G |
| Oncogene yuasa | | G |
| Oncostatin M | OSM | G |
| Oncostatin M receptor | OSMR | G |
| Orexin | OX | G |
| Orexin 1 receptor | OX1R | G |
| Orexin 2 receptor | OX2R | G |
| Orthodenticle (Drosophila) homolog 1 | OTX1 | G |
| Orthodenticle (Drosophila) homolog 2 | OTX2 | G |
| Osteonectin | ON | G |
| Osteopontin | OPN | G |
| Osteoprotegerin | OPG | G |
| p21-activated kinase 3 | PAK3 | G |
| Paired box homeotic gene 1 | PAX1 | G |
| Paired box homeotic gene 2 | PAX2 | G |
| Paired box homeotic gene 3 | PAX3 | G |
| Paired box homeotic gene 6 | PAX6 | G |
| Paired box homeotic gene 7 | PAX7 | G |
| Paired box homeotic gene 8 | PAX8 | G |
| Paired-like homeodomain transcription | PITX2 | G |
| factor 2 | | |
| Paired-like homeodomain transcription | PITX3 | G |
| factor 3 | | |
| Parathyroid hormone | PTH | G |
| Parathyroid hormone receptor | PTHR1 | G |
| Parathyroid hormone related-peptide | PTHrP | G |
| Parathyroid hormone-like hormone | PTHLH | G |
| Parvalbumin | PVALB | G |
| Patched (Drosophila) homolog, PTCH | PTCH | G |
| Phosphatase & tensin homolog | PTEN | G |
| Phosphate regulating gene with | PHEX | G |
| homologies to endopeptidases on the | | |
| X chromosome | | |
| Phosphatidylinositol glycan, class A | PIGA | G |
| (paroxysmal nocturnal hemoglobinuria) | | |
| Phosphatidylinositol transfer protein | PITPN | G |
| Phosphodiesterase 1/nucleotide | PDNP1 | G |
| pyrophosphatase 1 | | |
| Phosphodiesterase 1/nucleotide | PDNP2 | G |
| pyrophosphatase 2 | | |
| Phosphodiesterase 1/nucleotide | PDNP3 | G |
| pyrophosphatase 3 | | |
| Phosphomannomutase 1 | PMM1 | G |
| Phosphomannomutase 2 | PMM2 | G |
| Phytanoyl-CoA hydroxylase | PHYH | G |
| Platelet derived growth factor | PDGF | G |
| Platelet derived growth factor receptor | PDGFR | G |
| Poly(A) binding protein 2 | PABP2 | G |
| POU domain, class 1, transcription | POU1F1 | G |
| factor 1 (Pit1) | | |
| POU domain, class 3, transcription | POU3F4 | G |
| factor 4 | | |
| POU domain, class 4, transcription | POU4F3 | G |
| factor 3 | | |
| Pre-B-cell leukemia transcription factor 1 | PBX1 | G |
| Preproglucagon | GCG; GLP1; | G |
| GLP2 | | |
| Profibrinolysin | | G |
| Progesterone receptor (RU486 binding | PGR | G |
| receptor) | | |
| Prohibitin | PHB | G |
| Prolactin | PRL | G |
| Prolactin receptor | PRLR | G |
| Prolactin releasing hormone | PRH | G |
| Proliferin | PLF | G |
| Pro-melanin-concentrating hormone | PMCH | G |
| Promyclocytic leukemia gene | PML | G |
| Prophet of Pit1 | PROP1 | G |
| Prostaglandin (PG) D synthase, | PGDS | E |
| hematopoietic | | |
| Prostaglandin isomerase | | G |
| Prostaglandin-endoperoxidase synthase 2 | PTGS2 | G |
| Prostate cancer anti-metastasis gene KAI1 | KAI1 | G |
| Protein tyrosine phosphatase, non-receptor | PTPN12 | G |
| type 12 | | |
| RAD51, DNA repair protein | RAD51 | G |
| RAD52, DNA repair protein | RAD52 | G |
| RAD54, DNA repair protein | RAD54 | G |
| RAD55, DNA repair protein | RAD55 | G |
| RAD57, DNA repair protein | RAD57 | G |
| Ras-G-protein | RAS | G |
| Rathke pouch homeobox, RPX | RPX | G |
| Receptor tyrosine kinase (RTK), Nsk2 | NSK2 | G |
| Recombination activating gene 1 | RAG1 | G |
| Recombination activating gene 2 | RAG2 | G |
| Relaxin H1 | RLN1 | G |
| Relaxin H2 | RLN2 | G |
| Retinoblastoma 1 | RB1 | G |
| Retinoic acid receptor, alpha | RARA | G |
| Retinoic acid receptor, beta | RARB | G |
| Retinoic acid receptor, gamma | RARG | G |
| Retinoid X receptor, alpha | RXRA | G |
| Retinoid X receptor, beta | RXRB | G |
| Retinoid X receptor, gamma | RXRG | G |
| Retinoschisis, X-linked, juvenile | RS | G |
| Rhabdoid tumors | SMARCB1 | G |
| RIGUI | RIGUI | G |
| Ryanodine receptor 1, skeletal | RYR1 | G |
| SA homolog | SAH | G |
| Sal-like 1 | SALL1 | G |
| Serine/threonine kinase 11 | STK11 | G |
| Serine/threonine kinase 2 | STK2 | G |
| Sex determining region Y, SRY | SRY | G |
| Short stature homeobox | SHOX | G |
| Sialoprotein, bone | BSP | G |
| Signal transducer and activator of | STAT1 | G |
| transcription 1 | | |
| Signal transducer and activator of | STAT2 | G |
| transcription 2 | | |
| Signal transducer and activator of | STAT3 | G |
| transcription 3 | | |
| Signal transducer and activator of | STAT4 | G |
| transcription 4 | | |
| Signal transducer and activator of | STAT5 | G |
| transcription 5 | | |
| Sine oculis homeobox, drosophila, | SIX1 | G |
| homolog 1 | | |
| Sine oculis homeobox, drosophila, | SIX2 | G |
| homolog 2 | | |
| Sine oculis homeobox, drosophila, | SIX5 | G |
| homolog 5 | | |
| Slug protein | | G |
| Smoothelin | SMTN | G |
| Smoothened (Drosophila) homolog | SMOH | G |
| Somatotrophin | | G |
| Sonic hedgehog, SHH | SHH | G |
| SOS1 guanine nucleotide exchange factor | SOS1 | G |
| Spastic paraplegia 7 | SPG7 | G |
| Sperm adhesion molecule | SPAM1 | G |
| Sperm protamine P1 | PRM1 | G |
| Sperm protaminc P2 | PRM2 | G |
| Split hand/foot malformation gene | DSS1 | G |
| SRY-box 10 | SOX10 | G |
| SRY-box 11 | SOX11 | G |
| SRY-box 3 | SOX3 | G |
| SRY-box 4 | SOX4 | G |
| SRY-box 9 | SOX9 | G |
| Stem cell factor | SCF | G |
| Steroid hormone receptor responsive DNA | | G |
| elements | | |
| Stromal derived factor 1 | SDF1 | G |
| Sulfamidase | SGSH | G |
| Sulfonylurea receptor | SUR | G |
| Suppression of tumorigenicity 3 gene | ST3 | G |
| Suppression of tumorigenicity 8 gene | ST8 | G |
| Surfeit 1 | SURF1 | G |
| Syndecan 1 | SYND1 | G |
| Syndecan 2 | SYND2 | G |
| Syndecan 3 | SYND3 | G |
| Syndecan 4 | SYND4 | G |
| Synovial sarcoma gene 1 | SSX1 | G |
| Synovial sarcoma gene 2 | SSX2 | G |
| Talin | TLN | G |
| TATA binding protein | TBP | G |
| TATA binding protein associated | TAF2A | G |
| factor 2A | | |
| TATA binding protein associated | TAF2C2 | G |
| factor 2C2 | | |
| TATA binding protein associated | TAF2E | G |
| factor 2D | | |
| TATA binding protein associated | TAF2F | G |
| factor 2F | | |
| TATA binding protein associated | TAF2H | G |
| factor 2H | | |
| TATA binding protein associated | TAF2I | G |
| factor 2I | | |
| TATA binding protein associated | TAF2J | G |
| factor 2J | | |
| TATA binding protein associated | TAF2K | G |
| factor 2K | | |
| T-BOX 1 | TBX1 | G |
| T-BOX 2 | TBX2 | G |
| T-BOX 3 | TBX3 | G |
| T-BOX 4 | TBX4 | G |
| T-BOX 5 | TBX5 | G |
| T-BOX 6 | TBX6 | G |
| Testis-specific protein Y | TSPY | G |
| Thrombopoietin | THPO | G |
| Thrombospondin | THBS1 | G |
| Thymopoietin | TMPO | G |
| Thyroglobulin | TG | G |
| Thyroid hormone receptor, alpha | THRA | G |
| Thyroid hormone receptor, beta | THRB | G |
| Thyroid peroxidase | TPO | G |
| Thyroid receptor auxiliary protein | TRAP | G |
| Thyroid-stimulating hormone receptor | TSHR | G |
| Thyroid-stimulating hormone, alpha | TSHA | G |
| Thyroid-stimulating hormone, beta | TSHB | G |
| Thyrotroph embryonic factor | TEF | G |
| Thyrotropin releasing hormone | TRH | G |
| Thyrotropin releasing hormone receptor | TRHR | G |
| TIE receptor tyrosine kinase | TIE-1 | G |
| Torticollis, keloids, cryptorchidism and | TKCR | G |
| renal | | |
| dysplasia gene | | G |
| Transcription factor 1, hepatic | TCF1 | G |
| Transcription factor 2, hepatic | TCF2 | G |
| Transcription factor 3 | TCF3 | G |
| Transcription factor binding to IGHM | TFE3 | G |
| enhancer 3 | | |
| Transcription termination factor, RNA | TTF1 | G |
| polymerase | | |
| 1 | | G |
| Transcription termination factor, RNA | TTF2 | G |
| polymerase | | |
| 2 | | G |
| Transcription termination factor, RNA | TTF3 | G |
| polymerase | | |
| 3 | | G |
| Transferrin | TF | G |
| Transferrin receptor | TFRC | G |
| Transforming growth factor, alpha | TGFA | G |
| Transforming growth factor, beta 2 | TGFB2 | G |
| Transforming growth factor, beta induced | TGFB1 | G |
| Transforming growth factor, beta | TGFBR2 | G |
| receptor 2 | | |
| Transglutaminase 1 | TGM 1 | G |
| Transglutaminase 2 | TGM2 | G |
| Transglutaminase 4 | TGM4 | G |
| Translocation in renal carcinoma on | TRC8 | G |
| chromosome 8 | | |
| gene | G | |
| Treacle gene | TCOF1 | G |
| Tubby-like protein 1 | TULP 1 | G |
| Tuberous sclerosis 1 | TSC1 | G |
| Tuberous sclerosis 2 | TSC2 | G |
| Tumor susceptibility gene 101 | TSG101 | G |
| Tumour protein p53 | TP53, P53 | G |
| Tumour protein p63 | TP63 | G |
| Tumour protein p73 | TP73 | G |
| Tumour protein, translationally- | TPT1 | G |
| controlled 1 | | |
| Twist (Drosophila) homolog | TWIST | G |
| Ubiquitin | | G |
| Ubiquitin B | UBB | G |
| Ubiquitin C | UBC | G |
| Ubiquitin carboxyl-terminal esterase L1 | UCHL1 | G |
| Ubiquitin fusion degeneration 1-like | UFD1L | G |
| Vascular endothelial growth factor | VEGF | G |
| Vasoinhibitory peptide | | G |
| Vitamin B12-binding (R) protein | | G |
| Vitamin D receptor | VDR | G |
| v-myc avian myclocytomatosis viral | MYC | G |
| oncogene | | |
| homolog | | G |
| Von Hippel-Lindau gene | VHL | G |
| Werner syndrome helicase | WRN | G |
| Wilms tumour gene 1 | WT1 | G |
| Wilms tumour gene 2 | WT2 | G |
| Wilms tumour gene 4 | WT4 | G |
| Winged helix nude | WHN | G |
| Wingless family, wnt2 | WNT2 | G |
| Wingless family, wnt4 | WNT4 | G |
| Wingless family, wnt5 | WNT5 | G |
| Wingless family, wnt7 | WNT7 | G |
| Wingless family, wnt8 | WNT8 | G |
| Wnt inhibitory factor, WIF-1 | WIF1 | G |
| Wolf-Hirschhorn syndrome candidate | WHSC1 | G |
| 1 gene | | |
| X (inactive)-specific transcript | XIST | G |
| X-ray repair gene | XRCC9 | G |
| YY1 transcription factor | YY1 | G |
| Zona pellucida glycoprotein 1 | ZP11 | G |
| Zona pellucida glycoprotein 2 | ZP2 | G |
| Zona pellucida glycoprotein 3 | ZP3 | G |
| Zona pellucida receptor tyrosine kinase | ZRK | G |
| Zonadhesin | ZAN | G |

KEY TO ‘PROTEIN FUNCTION’ COLUMN
E ENZYME
T TRANSPORT & STORAGE
S STRUCTURAL
I IMMUNITY
N NERVOUS TRANSMISSION
G GROWTH & DIFFERENTIATION

2. A set of probes, said probes being antibodies or antibody fragments which interact with specific expressed proteins encoded by gene sequences of a group of genes, said probes being for detecting relevant variants (mutations and polymorphisms), e.g. nucleotide substitutions (missense, nonsense, splicing and regulatory), small deletions, small insertions, small insertion deletions, gross insertions, gross deletions, duplications, complex rearrangements and repeat variations in a target group of genes; characterised in that said group is a core group of genes consisting of substantially all of the genes defined in claim 1.

3. A set according to claim 1 or 2 in which a minority of said probes for listed genes are absent.

4. A set according to claim 1 or 2 in which a limited number of additional probes are present together with substantially all of the probes for the listed genes.

5. A set according to claim 1 or 2 in which a limited number of probes are replaced by probes for non-listed genes.

6. A set of probes for a core group of genes according to any of claims 1 to 5 in which each gene to be probed is substantially similar (greater than 85% homologous) in sequence to the respective member of the core list of genes.

7. A set according to any of claims 1 to 6 consisting of probes for members of a sub-group of the core group.

8. A set according to any preceding claim in which said probes are in the form of an array and are spatially arranged at known locations on a substrate.

9. A set according to any preceding claim wherein said probes are on a substrate which forms part of or consists of one or more chip plate(s), for use in a chip assay for detection of said gene variants.

10. A set according to any preceding claim in which said probes are mass, electrostatic or fluorescence tagged probes.

11. A set according to claim 8 or 9 in which said substrate is a semiconductor microchip.

12. A set according to any preceding claim for use in a biological assay for detection of said gene variants.

13. A set according to any preceding claim for use in the measurement of differential gene expression levels.

14. A medical device including a set according to any preceding claim for use in an assay for detection of said gene variants.

15. A medical device including a set according to any of claims 1 to 13 for use in an array for detection of differential gene expression levels.

16. A method for use in assessing the genomic profile of a patient or individual, the method comprising testing for and detecting the presence or absence of DNA or RNA encoding the relevant structural variants (as defined in claim 1) in a target group of genes by hybridising a nucleic acid-containing sample from said patient or individual to a set according to any of claims 1 and 3 to 13 and relating the probe hybridisation pattern to said variations.

17. A method for use in assessing the the genomic profile of a patient or individual, the method comprising testing for and detecting the presence or absence of DNA or RNA encoding the relevant structural variants (as defined in claim 2) in a target group of genes by interacting an expressed-protein-containing sample from said patient or individual with a set of probes according to any of claims 2 to 13 and relating the probe interaction pattern to said variations.

18. Use of a set or device according to any of claims 1 to 13 for the prognosis and management of patients suffering from or at risk of disease.

19. Use of a set or device according to any of claims 1 to 13 for predicting likely therapeutic response and adverse events following therapeutic intervention.

20. Use of a set or device according to any of claims 1 to 13 for predicting likely patterns of symptom clusters (symptom profiles) in disease and the likelihood of subsequent, contingent, disease or symptoms.

21. Use of a set or device according to any of claims 1 to 13 for general health screening, occupational health purposes, healthcare planning on a population basis and other healthcare management utilisations.

22. Use of a set or device according to any of claims 1 to 13 for the development of new strategies of therapeutic intervention and in clinical trials.

23. Use of a set or device according to any of claims 1 to 13 for construction of and generation of algorithms for patient and healthcare management.

24. Use of a set or device according to any of claims 1 to 13 for modelling or assessing the impact of diseases or healthcare management strategies on individuals, groups, patient cohorts or populations.

25. Use of a set or device according to any of claims 1 to 13 for modelling, assessing or exploring the theoretical impact of diseases and healthcare management strategies on individuals, groups, patient cohorts or populations.

26. Use of a set or device according to any of claims 1 to 13 for predicting optimum configuration/management of thereapeutic intervention.

27. A method according to claim 16 or 17 in which the identification of gene variants is indicative of a higher risk of developing clinical symptoms for the patient or individual.

28. A method for generating a model to assess whether a patient or individual or population or group is or are likely to develop clinical symptoms which method comprises:
i) obtaining DNA or RNA or protein samples from patients or individuals diagnosed as suffering from symptoms;
ii) obtaining DNA or RNA or protein samples from a control group of subjects diagnosed as not suffering from the symptoms;
iii) analysing the samples obtained in i) and ii) to identify the polymorphic variations encoded in the core group of genes as defined in any of claims 1 to 7;
iv) calculating the frequencies of these alleles in the samples from i) and ii);
v) comparing the frequencies of these alleles in i) and ii);
vi) performing a statistical analysis on the results from v) in order to generate a model for assessing the risk of developing symptoms.

29. A method for assessing whether a given subject will be at risk of developing symptoms, which comprises comparing said subject's genotype with a model generated by the method of claim 28.

30. A method according to any of claims 16, 17, 28 and 29 wherein at least one step is computer-controlled.

31. An assay suitable for use in a method according to any of claims 16, 17, 28 and 29; said assay comprising means for determining the presence or absence of relevant polymorphic variants of the core group of genes as defined in any of claims 1 to 7 in a biological sample.

32. A formatted assay technique (kit) for use in assessing the risk of a patient or individual developing symptoms; said kit comprising:
i) means for testing for the presence or absence or DNA or RNA encoding relevant polymorphic variants of the core group of genes as defined in claim 1 or 3 to 7 in a sample of human DNA;
ii) reagents for use in the detection process
iii) readout indicating the probability of a patient or individual developing symptoms.

33. A formatted assay technique (kit) for use in assessing the risk of a patient or individual developing symptoms; said kit comprising:
i) means for testing for the presence or absence of proteins encoded by the core group of genes and/or relevant polymorphic variants of the core group of genes as defined in any of claims 2 to 7 in an expressed-protein-containing human sample;
ii) reagents for use in the detection process
iii) readout indicating the probability of a patient or individual developing symptoms.

34. A set of probes according to claim 1, wherein the probes are selected from the group consisting of oligonucleotides and polynucleotides.
